(19) United States
(12) Patent Application Publication
(10) Pub. No.: US 2026/0021068 A1
(43) Pub. Date: Jan. 22, 2026

(54) METHODS AND COMPOSITIONS FOR INHIBITION OF TYROSINE AND PHENYLALANINE-MEDIATED DNA DAMAGE AND REPAIR

(71) Applicants: University of South Carolina, Columbia, SC (US); Functional Longevity Labs, Easton, MD (US)

(72) Inventors: Sajish Mathew, Lexington, SC (US); William K. Smith, Easton, MD (US)

(21) Appl. No.: 19/342,090

(22) Filed: Sep. 26, 2025

Related U.S. Application Data
(63) Continuation-in-part of application No. PCT/US2025/022449, filed on Apr. 1, 2025.
(60) Provisional application No. 63/572,516, filed on Apr. 1, 2024.
(60) Provisional application No. 63/699,404, filed on Sep. 26, 2024.
(60) Provisional application No. 63/699,513, filed on Sep. 26, 2024.

(51) Int. Cl. A61K 31/24; A61K 31/135; A61K 31/166; A61K 38/26; A61P 25/28; C07C 237/32

(52) U.S. Cl. CPC A61K 31/24; A61K 31/135; A61K 31/166; A61K 38/26; A61P 25/28; C07C 237/32

(57) ABSTRACT

Disclosed are compounds, compositions, and methods for inhibiting tyrosine-mediated DNA repair in a neuroprotective manner and/or activating transcription and/or protein synthesis. Compositions include a resveratrol, such as cis-resveratrol, or a compound of Formula I or Formula II. Compounds of Formula I and Formula II have the following structures

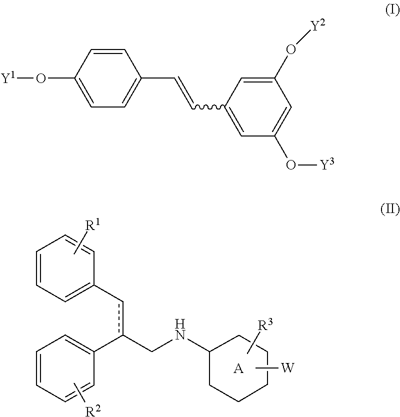

where the variables, e.g., Y¹, Y², Y³, R¹, R², R³ the A-ring, and W are defined herein. Said compound and compositions may be utilized in treating aging and age-associated neurocognitive and metabolic disorders including various types of cancer. In particular, compositions disclosed herein are neuroprotective against tyrosine/phenylalanine or their metabolites-mediated neurodegeneration and neurocognitive disorders. The disclosure also includes combination pharmaceutical compositions and methods of treatment in which cis-resveratrol, a compound of Formula I, a compound of Formula II, or a pharmaceutically acceptable salt thereof is used in combination with a GLP-1 receptor agonist for treating a traumatic brain injury or a neurodegenerative disorder.

### CROSS REFERENCE TO RELATED APPLICATION

This application is a continuation filed under 35 USC § 111(a) of international application PCT/US2025/022449, filed Apr. 1, 2025, which claims priority of U.S. Provisional Application No. 63/572,516, filed Apr. 1, 2024, and all the benefits accruing therefrom under 35 USC § 119, each of which is hereby incorporated by reference in its entirety. This application also claims priority of U.S. Provisional Appl. No. 63/699,404 filed Sep. 26, 2024 and of U.S. Provisional Appl. No. 63/699,513 filed Sep. 26, 2024, and all the benefits accruing therefrom under 35 USC § 119, each of which is hereby incorporated by reference in its entirety.

### GOVERNMENT SUPPORT CLAUSE

This invention was made with government support under grant P20 GM109091 awarded by National Institutes of Health (NIH). The government has certain rights in the invention.

### BACKGROUND

De novo protein synthesis is essential for the reactivation of the paused/stalled transcription complex to restore transcription. Stalled transcription complex is a hallmark of aging and age-associated disorders. Activation of protein synthesis consistently restores cognitive functions and memory formation in Alzheimer's Disease (AD) mice models. Aminoacyl-tRNA synthetases activate amino acids for protein synthesis. For example, tyrosyl-tRNA (TyrRS) activates tyrosine for protein synthesis. Recently, studies found that AD-affected brain regions show decreased tyrosyl-tRNA synthetase (TyrRS) and histone serine-ADP-ribosylation levels.

Protein synthesis is required for long-term memory formation. Tyrosine inhibits protein synthesis at the elongation step to deplete neuronal TyrRS and cause neuronal damage. Tyrosine is circadian regulated, with the highest serum levels in the morning and with the lowest around midnight (sleep time). Intriguingly, brain protein synthesis, memory formation, and neuronal DNA repair are activated during sleep when tyrosine levels are decreased during the nadir/trough of circadian rhythm. Tyrosine levels are also modulated by the circadian activities of tyrosine hydroxylase, tyrosine aminotransferases (TAT), and gut microbiota. Genetic mutations that increase the levels of tyrosine (tyrosinemia) or its precursor L-phenylalanine (Phe, phenylketonuria [PKU]) cause multiple health problems, including cognitive deficits in children. Moreover, tyrosine and/or phenylalanine exacerbate cognitive decline in the elderly and in Alzheimer's disease (AD) patients, in addition to shortening lifespan in tyrosinemia patients.

Increased tyrosine levels decrease TyrRS and cause neuronal oxidative DNA damage by simultaneously inhibiting protein synthesis and transcription along with stimulation of DNA repair and neurite degeneration. Dopamine and brain-derived neurotrophic factor (BDNF) increase TyrRS and histone serine-ADP-ribosylation.

Furthermore, cis-resveratrol (cis-RSV) binds to TyrRS mimicking a ‘tyrosine-free’ conformation, increases TyrRS, facilitates histone serine-ADP-ribosylation-dependent DNA repair, and provides neuroprotection in a TyrRS-dependent manner. Conversely, trans-RSV that binds to TyrRS mimicking a ‘tyrosine-like’ conformation decreases TyrRS, inhibits serine-ADP-ribosylation-dependent DNA repair, and induces neurodegeneration in rat cortical neurons. Thus cis-RSV protects the neurons against tyrosine and other neurotoxic agents-induced depletion of TyrRS and oxidative DNA damage and neurodegeneration.

The need exists for compounds, compositions, and methods useful for activating neuronal protein synthesis and transcription. Such compounds and compositions can act by increasing TyrRS levels and inhibiting tyrosine and phenylalanine-mediated neuronal DNA repair. Improvements in transcription may restore the function of crucial neuroinflammatory factors associated with cognitive performance and memory formation in neurodegenerant brains. In particular, a need exists for compounds, compositions, and methods for inhibiting tyrosine/phenylalanine-mediated neuronal DNA repair, and associated transcription inhibition by increasing TyrRS and activating protein synthesis in persons with autism spectrum disorders (ASD), in aging patients, in patients with age-associated neurocognitive and metabolic disorders including cardiovascular diseases (CVDs) and cancer. This disclosure addresses this need and provides other benefits which are detailed herein.

### SUMMARY

The present disclosure is directed to compositions and methods for inhibiting tyrosine-mediated DNA repair, methods for activating transcription, and methods for activating protein synthesis.

This disclosure provides a compound of Formula I

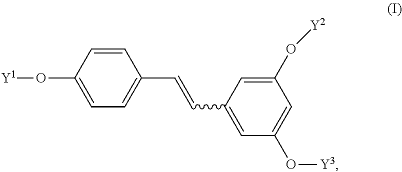

or a pharmaceutically acceptable salt thereof.

Within Formula I the variables, e.g., Y¹, Y², and Y³ have the following values. Any of the variable definitions set forth in this disclosure can be combined so long as a stable compound results.

The bond [symbol] indicates the compound of Formula I can be in either the cis or trans conformation or a mixture of cis and trans conformations.

Each of Y¹, Y², and Y³ is independently selected from a H or a group

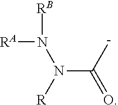

R is independently chosen at each occurrence from the 20 native amino acid side chains where any hydroxyl group in the amino acid side chain is optionally acylated with a C₂-C₆acyl group and when R is a proline the proline pyrrolidine ring is formed by R and R^{A} being joined by a —CH₂CH₂CH₂— chain.

R^{A} and R^{B} are independently chosen at each occurrence from H, C₂-C₆acyl, C₁-C₆alkyl, (C₃-C₆cycloalkyl)C₀-C₄alkyl, phenyl, and benzyl.

This disclosure also provides compounds of Formula II:

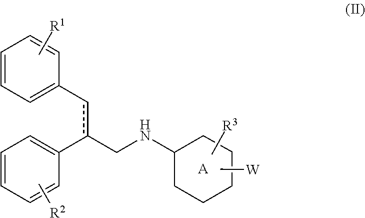

and the pharmaceutically acceptable salts thereof.

Within Formula II the following conditions are met.

The bond [symbol] is a double or single bond.

The A ring

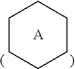

is phenyl, C₃-C₆cycloalkyl, or a 4- to 6-membered heterocyclic

group.

W is

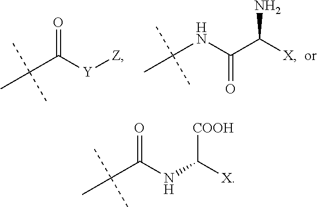

R¹ is absent or is 1 to 5 substituents independently selected from halogen, hydroxyl, amino, cyano, C₁-C₄alkyl, C₁-C₄alkoxy, (C₃-C₆cycloalkyl)C₀-C₂alkyl, C₁-C₂haloalkyl, and C₁-C₂haloalkoxy.

R² is absent or is 1 to 5 substituents independently selected from halogen, hydroxyl, amino, cyano, C₁-C₄alkyl, C₁-C₄alkoxy, (C₃-C₆cycloalkyl)C₀-C₂alkyl, C₁-C₂haloalkyl, and C₁-C₂haloalkoxy.

R³ is absent or is 1 to 4 substituents independently selected from halogen, C₁-C₂alkyl, C₁-C₂alkoxy, cyclopropyl, C₁-C₂haloalkyl, and C₁-C₂haloalkoxy.

Y is NH or O.

Z is H, hydroxyl, methyl, proline where the proline pyrrolidine ring is formed by Z and the nearest NH being joined by a —CH₂CH₂CH₂— chain, or Z is a group —CH₂X.

X is an amino acid side chain selected from H, —CH₃, —CH₂SH, —CH₂CO₂H, —CH₂OH, —CH(CH₃)OH, —CH₂CH₂CO₂H, —CH₂CH₂CONH₂,

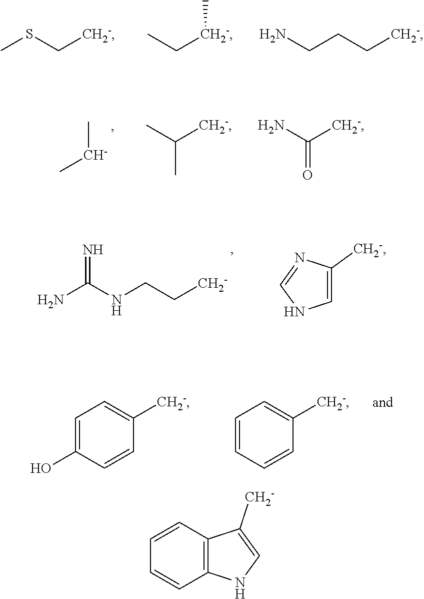

where a dash (—) indicates the point of attachment in Formula II.

The disclosure provides a method for inhibiting tyrosine-mediated DNA repair, the method comprising contacting a cell with an effective amount of a compound of Formula I or Formula II or a pharmaceutically acceptable salt of a compound Formula I or Formula II.

The disclosure also provides a method of treating a disorder in a subject, such as a neurocognitive disorder or a metabolic disorder, the method comprising administering a therapeutically effective amount of a compound of Formula I or Formula II or a pharmaceutically acceptable salt of Formula I or Formula II.

The disclosure includes pharmaceutical dosage forms comprising a compound of Formula I or Formula II or a pharmaceutically acceptable salt of Formula I or Formula II.

Compounds and compositions disclosed herein may be utilized in treating aging and age-associated neurocognitive and metabolic disorders. In particular, compositions disclosed herein are neuroprotective by inhibiting tyrosine-mediated DNA repair to activate transcription or inhibiting phenylalanine-mediated depletion of TyrRS and activation of inflammatory response. Similarly, compositions disclosed herein are neuroprotective by inhibiting tyrosine and phenylalanine combination-mediated neurite degeneration as well.

The disclosure also includes a pharmaceutical dosage form comprising
(i) cis-resveratrol, a compound of Formula I or Formula II or a pharmaceutically acceptable salt of any of the foregoing; and
(ii) a GLP-1 receptor agonist or pharmaceutically acceptable salt thereof, where the GLP-1 agonist selected from liraglutide, semaglutide, dulaglutide, exenatide, lixisenatide, tirzepatide, danuglipron (CAS Reg. No. 2230198-02-2), and orforglipron (CAS Reg. No. 2212020-52-3).

A method of treating a traumatic brain injury, a neurodegenerative disorder, or age-related cognitive impairment, the method comprising administering to a patient in need of such treatment
(i) a therapeutically effective amount of cis-resveratrol, a compound of Formula I, or a compound of Formula II, or a pharmaceutically acceptable salt thereof of the foregoing; and
(ii) a therapeutically effective amount of a GLP-1 receptor agonist or pharmaceutically acceptable salt thereof, where the GLP-1 agonist selected from liraglutide, semaglutide, dulaglutide, exenatide, lixisenatide, tirzepatide, danuglipron (CAS Reg. No. 2230198-02-2), and orforglipron (CAS Reg. No. 2212020-52-3).

A pharmaceutical kit comprising a package containing
(i) one of a dosage for comprising a dosage form of cis-resveratrol, a compound of Formula I, a compound of Formula II, or a pharmaceutically acceptable salt of any of the foregoing; and
(ii) a dosage form of a GLP-1 receptor agonist or pharmaceutically acceptable salt thereof, where the GLP-1 agonist selected from liraglutide, semaglutide, dulaglutide, exenatide, lixisenatide, tirzepatide, danuglipron (CAS Reg. No. 2230198-02-2), and orforglipron (CAS Reg. No. 2212020-52-3).

### BRIEF DESCRIPTION OF THE DRAWINGS

A full and enabling disclosure of the present disclosure is set forth more particularly in the remainder of the specification, including reference to the accompanying figures, in which:

FIG. 1A presents spectral images and quantitative IF analysis of TyrRS in the nucleus, soma, and neurites of primary cortical neurons (DIV 9/10) using anti-TyrRS antibody after treatment with thiorphan (100 nM) for 2 hr or verubecestat (50 nM) for 4 hr.

FIG. 1B presents spectral images and quantitative IF analysis of TyrRS in the nucleus, soma, and neurites of rat cortical neurons (DIV 9/10) using anti-TyrRS antibody after treatment with Aβ40 and Aβ42 (50 pM) for 4 hr.

FIG. 1C presents spectral images and quantitative IF analysis of TyrRS in the nucleus, soma, and neurites of rat cortical neurons (DIV 9/10) using anti-TyrRS antibody after treatment with Aβ42 (50 pM) alone or in combination with tyrosine (500 μM) for 4 hr.

FIG. 1D presents spectral images and quantitative IF analysis of TyrRS in the nucleus, soma, and neurites of rat cortical neurons (DIV 9/10) using anti-TyrRS antibody after treatment with Aβ40 (50 pM) alone or in combination with tyrosine (500 μM) for 4 hr.

FIG. 2A presents primary cortical neurons treated with thiorphan (50-500 nM) for 2 hr and TyrRS and PherRSα/β levels were detected by WB using their specific antibodies.

FIG. 2B presents spectral images and quantitative IF analysis of TyrRS in the nucleus, soma, and neurites of rat cortical neurons (DIV 9/10) using anti-TyrRS antibody after treatment with Aβ42 (50 pM) either alone or in combination with emetine (Em) for 4 hr.

FIG. 2C presents spectral images and quantitative IF analysis of TyrRS in the nucleus, soma, and neurites of rat cortical neurons (DIV 9/10) using anti-TyrRS antibody after treatment with Aβ40 (50 pM) either alone or in combination with anisomycin (Ani) for 4 hr.

FIG. 2D presents western blot image of primary cortical neurons were treated with reverse peptide of Aβ42 (42-1) (50 pM) for 2 hr and TyrRS levels were detected by WB.

FIG. 2E presents spectral images of spectral images and quantitative IF analysis of TyrRS in the nucleus, soma, and neurites of rat cortical neurons (DIV 9/10) using anti-TyrRS antibody after treatment with Aβ40 and Aβ42 (50 PM) for 1 hr.

FIG. 2F presents western blot images of primary rat cortical neurons treated with thiorphan (250 nM) for up to 16 hr and TyrRS and TrpRS levels detected by WB using their specific antibodies.

FIG. 3A presents western blot images of primary rat cortical neurons treated with increasing concentrations of thiorphan (100-500 nM) for 2 hr, followed by puromycin incorporation and western blot (WB) detection using an anti-puromycin antibody.

FIG. 3B presents primary cortical neurons treated with thiorphan (100 nM) for 2 hr and polysomes were separated using a sucrose density gradient ultracentrifugation and the ribosomal contents were measured in each fraction at wavelength 254.

FIG. 3C presents primary cortical neurons treated with Aβ40 and Aβ42 (50 pM) for 1 hr, followed by puromycin incorporation and detection using an anti-puromycin antibody.

FIG. 3D presents primary cortical neurons treated with verubecestat (50 nM) for 2 hr, followed by puromycin incorporation and detection using anti-puromycin antibody.

FIG. 3E presents primary cortical neurons treated with thiorphan (50-250 nM) for 2 hr and p-eIF2α and p-eEF2 levels were detected by WB analysis using anti-p-eIF2α, anti-p-eEF2, anti-eIF2α, and anti-eEF2 antibodies, respectively.

FIG. 3F presents primary cortical neurons treated with Aβ40 and Aβ42 (50 pM) for up to 15 min and p-eIF2α and p-eEF2 levels were detected by WB analysis using anti-p-eIF2α and anti-p-eEF2 antibodies, respectively.

FIG. 4A presents cortical neurons treated with tyrosine (500 μM) for up to 8 hr and the changes in PARP1, TOP1, MRE11, FEN1, and VCP levels were determined by WB using their corresponding antibodies.

FIG. 4B presents chromatin separation assay for cortical neurons treated with increasing concentrations of tyrosine (0.1-0.5 mM) for 15 min and association of TOP1, TOP2β, PARP1, PAR, FEN1, VCP, ARH3, TyrRS with the chromatin were determined using their specific antibodies.

FIG. 4C presents primary cortical neurons (DIV 9/10) treated with tyrosine (500 μM) for up to 16 hr and the percentages of DNA in the comet tail were quantified.

FIG. 4D presents primary cortical neurons (DIV 9/10) treated with phenylalanine (500 μM) for up to 16 hr and the percentages of DNA in the comet tail were quantified.

FIG. 4E presents primary cortical neurons (DIV 9/10) treated with tyrosine (500 μM) for 4 hr followed by a 30 min pulse labeling using 50 μM of nucleoside analog, CldU (5-chloro-2′-deoxyuridine).

FIG. 4F presents primary cortical neurons (DIV 9/10) treated with tyrosine (100-500 μM) for 8 hr and changes in the levels of TOP1, c-FOS, UBE3A, H3-Ser-ADPR, and γ-H2AX levels were determined by WB using their specific antibodies.

FIG. 4G presents primary cortical neurons (DIV 9/10) treated with phenylalanine (100-500 μM) for 8 hr and changes in the levels of TOP1, c-FOS, UBE3A, H3-Ser-ADPR, and γ-H2AX levels were determined by WB using their specific antibodies.

FIG. 5A presents graph showing the quantification of EU incorporation in nascent RNA. Primary cortical neurons were treated with treated with either tyrosine or phenylalanine (500 mM) for 16 hr followed by the addition of EU for 30 min and subsequent processing for image analysis as per the manufacturer's protocol.

FIG. 5B presents graph showing the quantification of 8-oxo-dG levels in primary cortical neurons treated with either tyrosine or phenylalanine (500 μM) for 16 hr.

FIG. 5C presents graph showing the quantification of EU incorporation in nascent RNA. Primary cortical neurons were treated with either Aβ40 (250 pM) or Aβ42 (100 pM) alone for 3 hr or in cultures pre-treated with tyrosine (500 μM) for 16 hr followed by the addition of EU for 30 min and subsequent processing for image analysis as per the manufacturer's protocol.

FIG. 5D presents graph showing the quantification of 8-oxo-dG levels in primary cortical neurons treated with either Aβ40 (250 pM) or Aβ42 (100 pM) alone for 3 hr or in cultures pre-treated with tyrosine (500 μM) for 16 hr.

FIG. 5E presents primary rat cortical neurons treated with Aβ40 and Aβ42 (50 pM) for 10 min and PARP1 was immunoprecipitated using anti-PARP1 antibody (α-PARP1) to determine the interaction of PARP1 with RNF4 by WB using anti-RNF4 antibody.

FIG. 5F presents primary cortical neurons (DIV 9/10) treated with Aβ40 (1 nM), Aβ42(250 pM) alone for 1 hr or in neuronal cultures pre-treated with tyrosine (500 μM) for 16 hr and the percentages of DNA in the comet tail were quantified.

FIG. 5G presents primary cortical neurons (DIV 9/10) treated with Aβ40 (1 nM), Aβ42 (250 pM) alone or in combination with tyrosine (500 μM) for 4 hr followed by a 30 min pulse labeling using 50 μM of the deoxynucleoside analog, CldU (5-chloro-2′-deoxyuridine).

FIG. 6A presents primary cortical neurons (DIV 9/10) treated with Aβ40 and Aβ42 (5, 10, and 25 pM) for 15 min and chromatin-associated proteins were separated using centrifugation. The presence of TOP1, TOP2β, FEN1, PARP1, PARylation, ARH3, p65/RelA, CHD1L, DDB1, and TyrRS in the pellet and supernatant fractions were detected by WB using their corresponding antibodies.

FIG. 6B presents primary rat cortical neurons (DIV 9/10) treated with Aβ40 and Aβ42 (10, 25, and 50 pM) for 4 hr and the changes in TOP1, PARP1, UBE3A, TyrRS, p65/RelA and CaMK2D levels were determined by WB using their respective antibodies.

FIG. 6C presents primary cortical neurons (DIV 9/10) treated with Aβ40 and Aβ42 (25 pM) for 10 min and TOP1 was immunoprecipitated using anti-TOP1 antibody. The levels of PARylation and its interaction with PARP1 and TyrRS were detected by immunoblot using anti-PAR, anti-PARP1, and anti-TyrRS antibodies respectively.

FIG. 6D presents primary cortical neurons (DIV 7) were transfected with siRNA against PARP1 (siRNA^{PARP1}), TyrRS (siRNA^{TyrRS}), or control siRNA (75 nM) for 72 hr and then treated with Aβ40 (1 nM) for 24 hr in combination with CPT (10 μM). Neuronal viability was assessed and quantified using MTT assay. The knockdowns of PARP1 and TyrRS were verified by WB.

FIG. 6E presents primary cortical neurons (DIV 9/10) treated with Aβ40 (1 nM), Aβ42 (250 pM), and CPT (10 μM) alone for 1 hr or in combination with CPT for 1 hr in neuronal cultures pre-treated with Aβ40 (1 nM) or Aβ42 (250 pM) for 2 hr and the percentages of DNA in the comet tail were quantified.

FIG. 7A-D presents picomolar Aβ42 protects against tyrosine-phenylalanine combination induced neurite degeneration whereas verubecestat exacerbates it. Representative images (scale bar, 20 μm) of primary cortical neurons following pre-treated with either tyrosine (500 μM), or phenylalanine (500 μM) or Tyr-Phe (0.5 mM each), Aβ40 or Aβ42 (50 pM), verubecestat (100 nM) alone for 20 hr before exposing them to Aβ40 or Aβ42 (50 pM) for another 4 hr. (MAP2—neurite marker, magenta and DAPI—nuclear marker, blue). Neurons were immunoassayed with anti-MAP2 antibody and quantified for neurite degeneration.

FIG. 8A presents primary cortical neurons were treated with CPT (10 μM) either alone or in combination with Aβ40 (1 nM) or Aβ42 (250 pM), thiorphan (500 nM) or verubecestat (50 nM) or tyrosine (250 μM) or phenylalanine (250 μM) for 24 hr and quantified neuronal survival using MTT assay.

FIG. 8B presents primary cortical neurons treated with ETO (10 μM) either alone or in combination with Aβ40 (1 nM), Aβ42 (250 pM), thiorphan (500 nM), verubecestat (50 nM), tyrosine (250 μM), or phenylalanine (250 μM) for 24 hr and quantified neuronal survival using MTT assay.

FIG. 8C presents primary rat cortical cultures treated with NMDA either alone or in combination with Aβ40 (250 pM), Aβ42 (250 pM), tyrosine (500 μM), or verubecestat (50 nM) and subjected them to MTT assay after 24 hr.

FIG. 8D illustrates mechanism of regulation of inflammation by tyrosine and phenylalanine by regulating neuronal DSBs and SSBs. γ-H2AX is a marker of DSBs and H3-Ser-ADPR is a marker of neuronal SSBs.

FIG. 9A presents western blot images showing that nanomolar concentrations of Aβ42 depletes TyrRS. Primary cortical neurons (DIV 9/10) were treated with Aβ42 (50 nM) for 8 hr and TyrRS and PheRSb protein levels were determined by WB analysis using their respective antibodies.

FIG. 9B presents western blot images showing that nanomolar concentrations of Aβ42 depletes TyrRS while accumulating PARP1. Primary cortical neurons (DIV 9/10) were treated with Aβ42 (50 nM) for 8 hr and PARP1, TOP1, and TyrRS protein levels were determined by WB analysis using their respective antibodies.

FIG. 9C presents western blot images showing that nanomolar concentrations of Aβ42 decreases histone serine-ADP-ribosylation. Primary cortical neurons (DIV 9/10) were treated with Aβ42 (50 nM) for 8 hr and H3-Ser-ADPR levels were determined by WB analysis using anti-H3-Ser-ADPR antibody.

FIG. 9D presents western blot images showing that nanomolar concentrations of Aβ40 depletes TyrRS while accumulating TOP1 protein. Primary cortical neurons (DIV 9/10) were treated with Aβ40 (50 nM) for 8 hr and PARP1, TOP1, and TyrRS protein levels were determined by WB analysis using their respective antibodies.

FIG. 9E presents quantitation of DNA damage levels in primary cortical neurons (DIV 9/10) showing that higher concentrations of amyloid beta fail to induce DNA breaks in presents of tyrosine. Cortical neurons were treated with nM Aβ40 (50 nM), nM Aβ42 (50 nM) alone for 1 hr or in neuronal cultures pre-treated with tyrosine (500 μM) for 16 hr and the percentages of DNA in the comet tail were quantified.

FIG. 10A presents spectral images and quantitative IF analysis of TyrRS in rat cortical neurons (DIV 9/10) using anti-TyrRS antibody after treatment with nM Aβ42 (50 nM) for 16 hr or in combination with BDNF for 1 hr showing that reactivation of protein synthesis rescues nM Aβ42-mediated TyrRS depletion.

FIG. 10B presents spectral images and quantitative IF analysis of TyrRS in rat cortical neurons (DIV 9/10) using anti-TyrRS antibody after treatment with nM Ab42 (50 nM) for 16 hr or in combination with A-484594 (eEF2K inhibitor, 100 nM) showing that reactivation of protein synthesis rescues nM Aβ42-mediated TyrRS depletion.

FIG. 10C presents spectral images and quantitative IF analysis of TyrRS in rat cortical neurons (DIV 9/10) using anti-TyrRS antibody after treatment with nM Ab42 (50 nM) for 16 hr or in combination with ISRIB (100 nM) showing that reactivation of protein synthesis rescues nM Ab42-mediated TyrRS depletion.

FIG. 10D presents spectral images and quantitative IF analysis of MAP2 in rat cortical neurons (DIV 9/10) after treatment with nM Aβ42 (50 nM) for 24 hr or in combination with ISRIB (100 nM) showing that reactivation of protein synthesis rescues nM Aβ42-mediated neurite degeneration.

FIG. 10E presents spectral images and quantitative IF analysis of γ-H2AX in rat cortical neurons (DIV 9/10) after treatment with nM Aβ42 (50 nM) for 24 hr or in combination with ISRIB (100 nM) showing that reactivation of protein synthesis rescues nM Aβ42-mediated DNA damage accumulation.

FIG. 11A presents graph showing the quantification of EU incorporation in nascent RNA demonstrating that cis-RSV protects against tyrosine-mediated inhibition of transcription. Primary cortical neurons were treated with treated with either tyrosine or cis-RSV (50 μM) for 16 hr followed by the addition of EU for 30 min and subsequent processing for image analysis as per the manufacturer's protocol.

FIG. 11B presents comet assay demonstrating that cis-RSV protects against tyrosine-mediated DNA repair. Primary cortical neurons (DIV 9/10) were treated with cis- and trans-RSV (50 μM) either alone or in combination with tyrosine (1 mM) for 16 hr and the percentages of DNA in the comet tail were quantified.

FIG. 11C presents primary rat cortical neurons treated with cis- and trans-RSV (50 μM) for up to 1 hr and the recruitment of TOP1 and TOP2β to the chromatin was determined after chromatin separation followed by WB using anti-TOP1 and anti-TOP2β antibodies, respectively.

FIG. 11D presents volcano blot showing the differential gene expression profile based on the RNA-Seq analysis in neurons treated with cis-RSV and trans-RSV for 24 hr.

FIG. 11E presents illustration of the Kyoto Encyclopedia of Genes and Genomes (KEGG) pathway analysis of cis-RSV downregulated mRNAs.

FIG. 12A illustrates Venn diagram showing the number of differentially expressed genes by cis- and trans-RSV based on the RNA-Seq analysis.

FIG. 12B presents illustration of the KEGG pathway analysis of cis-RSV upregulated mRNAs.

FIG. 12C presents illustration of the KEGG pathway analysis of trans-RSV downregulated mRNAs.

FIG. 12D presents illustration of the KEGG pathway analysis of trans-RSV upregulated mRNAs.

FIG. 13A presents gene ontology (GO) pathway analysis of cis-RSV downregulated mRNAs.

FIG. 13B presents GO pathway analysis of cis-RSV upregulated mRNAs.

FIG. 13C presents GO pathway analysis of trans-RSV downregulated mRNAs.

FIG. 13D presents GO pathway analysis of trans-RSV upregulated mRNAs.

Repeat use of reference characters in the present specification and drawings is intended to represent the same or analogous features or elements of the present invention.

FIG. 14. The effect of compound II-8 (TTC-369) on TyrRS (tyrosyl-tRNA synthetase), PheRSβ (phenylalanyl-tRNA synthetase beta), and PheRSα (phenylalanyl-tRNA synthetase alpha) levels in primary rat coritical neurons. FIG. 14A. Rat cortical neurons were treated with compound II-8 at concentrations of 0.1 nM 1.0 nM for 2 hours and then detecting TyrRS, PheRSβ, and PheRSα in the rat cortical neurons via Western blot using anti-TyrRS and anti-PheRS antibodies. FIG. 14B. TyrRS and PheRSβ levels as a function of increasing compound II-8 concentration. TyrRS and PheRSβ levels were increased in rat cortical neurons treated with II-8 at all tested concentrations between 0.1 nM to 1.0 nM relative to TyrRS and PheRSβ levels in untreated neurons.

FIG. 15. The effect of compound II-8 on PARylation in primary rat cortical neurons. FIG. 15A. Rat cortical neurons were treated with II-8 at concentrations from 0.25 to 2.5 nM for 15 minutes and then quantified PARylation levels in untreated and II-8 treated neurons via western blot analysis using anti-PAR antibody. FIG. 15B. II-8 treatment increased PARylation in rat cortical neurons relative to untreated rat cortical neurons at all tested concentrations. The maximum effect was at 1 nM.

FIG. 16. II-8 protects against tyrosine, NMDA, and etoposide (ETO)-induced DNA damage accumulation and neurotoxicity in rat cortical neurons. FIG. 16A. Rat cortical neurons were treated with 1 mM tyrosine either alone or in combination with II-8 (50 nM) for 16 hr and the quantified the changes in γ-H2AX foci levels (a marker of DNA damage) using immunofluorescent (IF) analysis. The number of γ-H2AX foci decreased slightly relative to the untreated group in response to II-8 treatment alone. The number of γ-H2AX foci increased approximately 3-fold relative to untreated in response to Tyrosine treatment but was similar to the untreated group when neurons were treated with both tyrosine and II-8. FIG. 16B. Quantification of number of γ-H2AX foci in II-8, Tyrosine only, and Tyrosine+II-8 treated groups.

FIG. 17. FIG. 17A. Rat cortical neurons treated with NMDA alone and II-8 alone and in combination at II-8 concentrations of 100 nM and 250 nM for 24 hours to assess the effect of II-8 on NMDA damage. The MTT assay was used to quantify neuronal viability. FIG. 17B. To test the effect of II-8 on etoposide mediated damage, rat cortical neurons were treated with ETO alone and II-8 alone and in combination at II-8 concentrations of 0.1 to 1 nM for 24 hours, again using the MTT assay to quantify neuronal viability. Treatment with NMDA alone or etoposide alone decreased neuronal viability. Treatment with II-8 in combination with NMDA or etoposide restored viability in a concentration dependent manner.

FIG. 18. II-8 protects against nanomolar amyloid beta 42 (Aβ42)-mediated neurite degeneration. Rat cortical neurons with either 50 nM Aβ42 alone (FIG. 18A) or in combination with 50 nM II-8 (FIG. 18B) for 24 hours. Quantification via the neurite degeneration index showed that treatment II-8 protects neurons from Aβ42 induced neurite degeneration.

FIG. 19. II-8 mimics cis-RSV in protecting against nanomolar amyloid beta 42 (Aβ42)-mediated neurotoxicity. Rat cortical neurons were treated with 50 nM Aβ42 or in combination with cis-RSV or trans-RSV or II-8 (100 pM, supplemented every 3^{rd} day) for 25 days and the neuronal viability was quantified using MTT assay.

FIG. 20. II-8 protects against tyrosine-mediated neuronal DNA repair. Primary cortical neurons (DIV 9/10) were treated with II-8 (1 nM) either alone or in combination with tyrosine (1 mM) for 16 hr and the percentages of DNA in the comet tail were quantified

FIG. 21. II-8 protects against tyrosine-mediated nucleotide incorporation in cortical neurons. Rat cortical neurons (DIV 9/10) were treated with CldU (100 μM) either alone or in combination with tyrosine (1 mM) or II-8 (1 nM) or their combination for 4 hr and nucleotide incorporation was determined using DNA fiber assay after staining with anti-CidU antibody using immunofluorescence (IF).

FIG. 22. shows an illustration of the mechanism of action of cis-RSV-mediated neuroprotection and trans-RSV-mediated neurotoxicity.

FIG. 23. shows at: (a) representative spectral images of the immunostaining (scale bar, 20 μm) and quantification (nuclear localization) for TyrRS protein in cortical neurons; (b) a graph of cell viability assessed and quantified using a MTT assay; and (c) shows a graph of viability of rat cortical neurons (DIV 8) treated with trans-RSV alone or in combination with different doses of cis-RSV (10-50 μM) for 48 hours.

FIG. 24 shows at: (a) a graph showing trans-RSV evoking dichotomic effects on neuronal survival under excitotoxicity; (b) a graph showing cis-RSV evoking dose-dependent neuroprotection against excitotoxicity; and (c) a graph showing only cis-RSV protects against MPP⁺ toxicity.

FIG. 25 shows at (d) representative images for cortical neurons following neurite degeneration.

FIG. 26 shows at: (a) representative immunoblots and quantification from chromatin fraction of cortical neurons (DIV 9) depicting PARP1 and PAR after treatment with cis- and trans-RSV (50 μM) for 1 hour; (b) representative immunoblots depicting effects of cis- and trans-RSV on inhibitory GSK3β phosphorylation (Ser 21/9) in cortical neurons (DIV 9) after treatment for 16 hours; and (c) representative spectral images of the immunostaining and quantification depicting effects of cis- and trans-RSV (25 μM) on phospho-tau (pSer404-tau (p-tau)) in cortical neurons (DIV 9) after treatment for 16 hours.

### DETAILED DESCRIPTION

### Terminology

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Compounds of the present disclosure are generally described using standard nomenclature.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The terms “a” and “an” do not denote a limitation of quantity, but rather denote the presence of at least one of the referenced item. The term “or” means “and/or.”

The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Formula I includes all subformulae and compounds that fall within the scope of Formula I and Formula II includes all subformulae and compounds that fall within the scope of Formula II.

In certain situations, the compounds of Formula I or Formula II may contain one or more asymmetric elements such as stereogenic centers, stereogenic axes and the like, e.g. asymmetric carbon atoms, so that the compounds can exist in different stereoisomeric forms. Unless stated otherwise or clearly indicated from the context, Formula I and Formula II include all stereoisomeric forms, including racemates, optically enriched, and optically pure forms. In addition, compounds with carbon-carbon double bonds may occur in Z- and E-forms, with all isomeric forms of the compounds being included in the present disclosure. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example, a chiral HPLC column.

Cis-resveratrol, Formula I, and Formula II includes all isotopically enriched versions of compounds of cis-resveratrol, Formula I, and Formula II. Isotopes include those atoms having the same atomic number but different mass numbers. By way of general example, and without limitation, isotopes of hydrogen include tritium and deuterium and isotopes of carbon include ¹¹C, ¹³C, and ¹⁴C, isotopes of fluorine include ¹⁹F, isotopes of nitrogen include ¹⁵N, and isotopes of oxygen include ¹⁷O. Isotopically enriched versions of compounds of cis-resveratrol, Formula I, and Formula II, include, e.g., compounds in which one or more C in cis-resveratrol, a compound of Formula I, or Formula II is 13C enriched, in which one or more hydrogen position in cis-resveratrol, a compound of Formula I, or Formula II is deuterated or tritiated, in which one or more N in cis-resveratrol, a compound of Formula I, or Formula II is ¹⁵N enriched.

Certain compounds are described herein using a general formula that includes variables, e.g., R¹, R², R³, Y¹, Y², Y³, R^{A}, R^{B}, R, W, X, Y, and Z. Unless otherwise specified, each variable within such a formula is defined independently of other variables. Thus, if a group is said to be substituted, e.g., with 0-2 R*, then said group may be substituted with up to two R* groups and R* at each occurrence is selected independently from the definition of R*.

Combinations of substituents and/or variables are permissible only if such combinations result in stable compounds. A stable compound or stable structure is meant to imply a compound that is sufficiently robust to survive isolation from a reaction mixture, and subsequent formulation into an effective therapeutic agent.

The term “substituted” means that any one or more hydrogen atoms bound to the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified substituents are named into the core structure.

Substituents are named into the ring unless otherwise indicated. A dash (“—”) or a double bond (“═”) that is not between two letters or symbols indicates the point of attachment for a substituent. For example, —CONH₂ is attached through the carbon atom.

The term “amino acid” as used herein refers to native amino acids, non-native amino acids, and amino acid analogs. Native amino acids include, for instance, the 20 (L)-amino acids commonly utilized during protein biosynthesis. Non-native amino acids include the 20 (D)-amino acids not utilized for protein biosynthesis, selenocysteine, and other plant-derived non-protecogenic amino acids such as pyrrolysine. Non-native amino acids include, for instance, selenocysteine, homoserine, ornithine, canvanine, N-methyl-L-alanine, L-DOPA (3,4-dihydroxyphenylalanine), Dap, and Dap. Amino acid analogs may include modified forms of naturally or non-naturally occurring amino acids, for instance, substitution or replacement of chemical groups and moieties on the amino acid or by derivatization of the amino acid. Pomaglumetad (LY-404,039) is an amino acid analog.

“Alkyl” includes both branched and straight-chain saturated aliphatic hydrocarbon groups, having the specified number of carbon atoms. Thus, the term C₁-C₆alkyl includes alkyl groups having from 1 to about 6 carbon atoms. When C₀-Cₙalkyl is used herein in conjunction with another group, for example, (cycloalkyl)C₀-C₂ alkyl, the indicated group, in this case cycloalkyl, is either directly bound by a single covalent bond (C₀), or attached by an alkyl chain having the specified number of carbon atoms, in this case from 1 to about 2 carbon atoms. Examples of alkyl include, but are not limited to, methyl, ethyl, n-propyl, isopropyl, n-butyl, t-butyl, n-pentyl, and sec-pentyl. C₁-C₄alkyl includes alkyl groups having 1, 2, 3, or 4 carbon atoms.

“Alkoxy” is an alkyl group as defined above with the indicated number of carbon atoms attached to the group it substitutes through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “Alkylthio” or a “thioalkyl” group is an alkyl group as defined above with the indicated number of carbon atoms covalently bound to the group it substitutes by a sulfur bridge (—S—).

“Acyl” is a group of the formula alkylC(═O)—, where alkyl has the definition given above. A C₂-C₆acyl group has an alkyl group of 1 to 5 carbon covalently bound to a keto (C═O) group which is in turn covalently bound to the group it substitutes.

“Halo” or “halogen” as used herein is fluoro, chloro, bromo, or iodo.

“Haloalkyl” includes both branched and straight-chain saturated aliphatic hydrocarbon groups, having the specified number of carbon atoms, substituted with 1 or more halogen atoms, generally up to the maximum allowable number of halogen atoms. Thus, the term C₁-C₆haloalkyl includes haloalkyl groups having from 1 to about 6 carbon atoms. Examples of haloalkyl include, but are not limited to, trifluoromethyl, difluoromethyl, 2-fluoroethyl, chloromethyl, chloroethyl, and penta-fluoroethyl. C₁-C₂alkyl includes alkyl groups having 1 or 2 carbon atoms, substituted with 1 or more halogen atoms, generally up to the maximum allowable number of halogen atoms.

“Haloalkoxy” is an haloalkyl group as defined above with the indicated number of carbon atoms attached through an oxygen bridge. Examples of haloalkoxy include, but are not limited to, chloromethoxy, chloroethoxy, bromo-n-propoxy, bromo-i-propoxy, iodo-n-butoxy, iodo-2-butoxy, or chloro-n-pentoxy.

A “heterocyclic group” is a monocyclic saturated, partially unsaturated, or aromatic ring containing from 1 to 4 heteroatoms chosen from N, O, and S, with remaining ring atoms being carbon, or a bicyclic saturated, partially unsaturated, or aromatic heterocycle containing at least 1 heteroatom chosen from N, O, and S in one of the two rings of the two ring system and containing up to about 4 heteroatoms independently chosen from N, O, and S in each ring of the two ring system. The rings of a bicyclic heterocyclic group can be in fused, bridged, pendant, or spiro orientation. Usually, each ring of the heterocycle contains from 4-6 ring atoms but some other number of ring atoms may be specified. Unless otherwise indicated, the heterocycle may be attached to its pendant group at any heteroatom or carbon atom that results in a stable structure. When indicated the heterocycles described herein may be substituted on carbon, sulfur, or nitrogen atom if the resulting compound is stable. It is preferred that the total number of heteroatoms in a heterocycle is not more than 4 and that the total number of S and O atoms in a heterocycle is not more than 2, more preferably not more than 1. Examples of heterocycles include, pyridyl, indolyl, pyrimidinyl, pyridazinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyrazolyl, pyrrolidinyl, morpholinyl, piperazinyl, piperidinyl, and pyrrolidinyl. In certain embodiments a heterocycle is chosen from pyridinyl, pyrimidinyl, furanyl, thienyl, and pyrrolyl.

Additional examples of heterocycles include, but are not limited to, phthalazinyl, indolizinyl, indazolyl, benzothiazolyl, benzimidazolyl, benzofuranyl, benzoisoxolyl, dihydro-benzodioxinyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanonyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, 5 pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromanyl, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrirnidinyl N-oxide, pyridazinyl N-oxide, pyrazinyl N-oxide, quinolinyl N-oxide, indolyl N-oxide, indolinyl N oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N oxide, indazolyl N-oxide, benzothiazolyl N-oxide, benzimidazolyl N-oxide, pyrrolyl N-oxide, oxadiazolyl N-oxide, thiadiazolyl N-oxide, tetrazolyl N-oxide, benzothiopyranyl S-oxide, and benzothiopyranyl S,S-dioxide.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Examples of heteroaryl groups include, but are not limited to, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

“Heterocycloalkyl” is a stable monocyclic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered cyclic ring which contains from 1 to 3, or in some embodiments from 1 to 2, heteroatoms chosen from N, O, and S, with remaining ring atoms being carbon. Monocyclic heterocycloalkyl groups typically have from 5 to 7 ring atoms. The stable monocyclic heterocycloalkyl may have 3-10 ring atoms which contains from 1 to 3, or in some embodiments from 1 to 2, heteroatoms chosen from N, O, and S, with remaining ring atoms being carbon. In some embodiments bicyclic heterocycloalkyl groups are 9- to 10-membered heterocycloalkyl groups, that is, groups containing 9 or 10 ring atoms in which one 5- to 7-member cyclic ring is fused to a second aromatic or non-aromatic ring. It is preferred that the total number of S and O atoms in the heteroaryl group is not more than 2. Examples of heterocycloalkyl groups include, but are not limited to, oxiranyl, oxetanyl, tetrahydrofuranyl, azetidinyl, pyrrolidinyl, piperidinyl, morpholinyl, or piperazinyl.

An “active agent” means a compound (including a compound disclosed herein), element, or mixture that when administered to a patient, alone or in combination with another compound, element, or mixture, confers, directly or indirectly, a physiological effect on the subject. The indirect physiological effect may occur via a metabolite or other indirect mechanism. The “active agent” may also potentiate or make more active another active agent.

The term “administering” includes all modes and routes of administration which allow a compound to perform its intended function. In general, the compounds disclosed herein may be administered to a subject according to known methods, including injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The compound may be administered alone, or in conjunction with a pharmaceutically acceptable carrier. The compound also may be administered as a prodrug, which is converted to its active form in vivo.

“Pharmaceutical compositions” are compositions comprising at least one active agent, such as a compound, salt, or hydrate of a named Formulac of the disclosure (Formula I or II) and at least one other excipient. “Excipients” are any materials of a pharmaceutical composition other than the active agent or agents. Excipients include carriers and diluents, which may be added to the pharmaceutical compositions. Pharmaceutical compositions meet the U.S. FDA's GMP (good manufacturing practice) standards for human or non-human drugs.

A “pharmaceutical dosage form” or “dosage form” is the physical form in which an active agent is prepared or administered. Examples of dosage forms include tablets, capsules, powders, and oral and injectable solutions. A “dose,” “unit dose,” or “dosage” Is a physically discrete unit suitable for use in a subject, each unit containing a predetermined quantity and/or a pharmaceutical formulation calculated to produce the desired response or responses in association with its administration.

The term “carrier” applied to pharmaceutical compositions/combinations of the invention refers to a diluent, excipient, or vehicle with which an active compound is provided. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

“Pharmaceutically acceptable salt” includes derivatives of the disclosed compounds wherein the parent compound is modified by making non-toxic acid or base salts thereof, and further refers to pharmaceutically acceptable hydrates or solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxylmaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002.

A “patient” or a “subject” is a human or non-human animal in need of medical treatment. Medical treatment can include treatment of an existing condition, such as a disease or disorder or diagnostic treatment. In some embodiments the patient is a human patient.

“Administering” means giving, providing, applying, or dispensing by any suitable route. Administration of a combination of active agents includes administration of the combination in a single formulation or unit dosage form, administration of the individual active agents of the combination concurrently but separately, or administration of the individual active agents of the combination sequentially by any suitable route. The dosage of the individual active agents of the combination may require more frequent administration of one of the active agent(s) as compared to the other active agent(s) in the combination. Therefore, to permit appropriate dosing, packaged pharmaceutical products may contain one or more dosage forms that contain the combination of active agents, and one or more dosage forms that contain one of the combination of active agents, but not the other active agent(s) of the combination.

“Treatment” or “treating” means providing an active compound to a patient in an amount sufficient to measurably reduce any existing condition or slow existing condition progression. In certain embodiments treatment can be preventative. Preventative treatment is treatment that reduces the likelihood of symptoms occurring, produces a statistically significant delay in symptoms occurring, or a statistically significant delay in symptoms occurring.

A significant change is any detectable change that is statistically significant in a standard parametric test of statistical significance such as Student's T-test, where p<0.05.

The term “combination therapy” refers to the administration of two or more therapeutic (active) agents to treat a condition or disorder described in the present disclosure. Such administration encompasses co-administration of these therapeutic agents in a substantially simultaneous manner, such as in a single dosage form having a fixed ratio of active ingredients or in separate dosage forms for each active ingredient. In addition, such administration also encompasses administration of each therapeutic agent in a sequential manner, cither at approximately the same time or at different times. In either case, the treatment regimen will provide the beneficial effects of each therapeutic agent in the drug combination in treating the conditions or disorders described herein.

The term “therapeutically effective amount” of a compound of the disclosure, means an amount effective, when administered to a patient, to provide a therapeutic benefit such as an amelioration of symptoms, e.g., an amount effective to decrease the symptoms of a neurocognitive or neurodegenerative disorder, and including an amount sufficient to reduce or inhibit DNA repair or an amount effect to decrease neuronal tyrosine levels. Thus, a therapeutically effective amount of a compound is also an amount sufficient to significantly reduce the indicia of the disease or condition being treated. A significant reduction is any detectable negative change that is statistically significant in a standard parametric test of statistical significance, such as Student's t-test, in which p<0.05.

### Chemical Description

Reference will now be made in detail to various embodiments of the presently disclosed subject matter, one or MORE examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is directed to compositions and methods to activate neuronal protein synthesis and transcription by inhibiting DNA repair. Improvements in transcription may restore the function of crucial neuroinflammatory factor associated with cognitive performance and memory formation in neurodegenerant brains. In particular, the present disclosure is directed to compositions and methods for inhibiting tyrosine/phenylalanine-mediated DNA repair and associated transcription inhibition by activating protein synthesis in aging and age-associated neurocognitive and metabolic disorders including ASD, CVDs, and cancer.

This disclosure further provides pharmaceutical dosage forms comprising (i) one of a cis-resveratrol analogue, such as a compound of Formula I or II or a pharmaceutically acceptable salt of Formula I or II and (ii) a GLP-1 receptor agonist.

Resveratrol (RSV) is a naturally occurring polyphenol, that occurs in cis- and trans-forms.

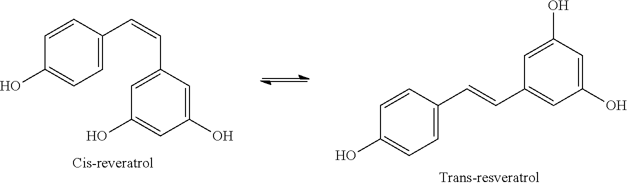 [SMILES: Oc1ccc(/C=C\c2cc(O)cc(O)c2)cc1 ; Oc1ccc(/C=C/c2cc(O)cc(O)c2)cc1]

Resveratrol has a tyrosine-like phenolic ring that mimics tyrosine in binding to the active site of TyrRS. Studies have shown that the sulfate metabolites of trans-RSV provide an intracellular pool to generate cis-RSV. Further, analysis of the x-ray crystal structures of TyrRS with and without cis-RSV showed that the binding of cis-RSV in the active site of TyrRS mimics its ‘tyrosine-free’ conformation. Thus, binding of cis-RSV may enable the moonlighting functions of TyrRS even in the presence of tyrosine. For example, D-tyrosine and trans-RSV, upon binding to TyrRS and mimicking a ‘tyrosine-like’ conformation, decrease TyrRS, inhibits DNA repair, and cause neurotoxicity by inhibiting HPF1/PARP1-dependent serine-ADP-ribosylation. Conversely, cis-RSV, upon binding to TyrRS and mimicking a ‘tyrosine-free’ conformation, increases TyrRS, facilitates DNA repair through serine-ADP-ribosylation, and provides neuroprotection in a TyrRS-dependent manner.

Only cis-RSV has been shown to protect neurons against stress conditions by activating TyrRS-regulated neuronal DNA repair and resilient signaling; trans-RSV, conversely, facilitated the downregulation of TyrRS resulting in the accumulation of DNA damage and subsequent neurodegeneration. Cis-RSV may be administered as isomerically pure cis-RSV or may be administered as a mixture of cis- and trans-resveratrol so long as the daily dose of trans-RSV does not exceed 200 mg for an adult human patient.

The disclosure provides resveratrol analogues, including cis-RSV analogues.

Compositions disclosed herein may include a DNA repair inhibitor, such as resveratrol or a derivative thereof.

For instance, the composition may include a resveratrol analogue of Formula I:

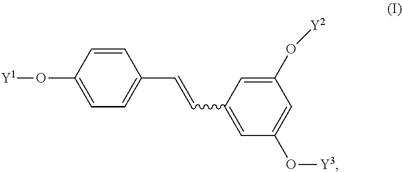

or a pharmaceutically acceptable salt thereof, wherein the variables, e.g., Y¹, Y², and Y³ carry the definitions set forth in the Summary section.

The bond, [symbol] , indicates the compound of Formula I can be in either the cis or trans conformation or a mixture of cis and trans conformations. The cis and trans conformations are

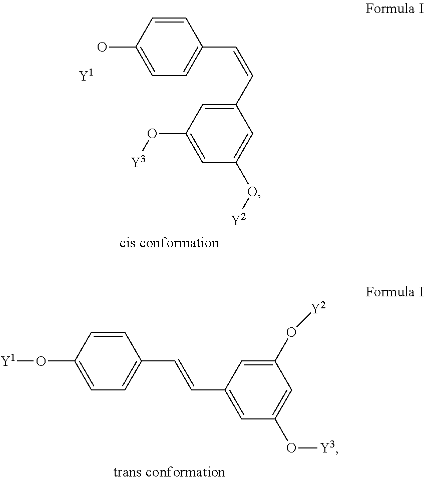

In certain embodiments the compound of Formula I, or salt thereof, is in the cis conformation.

The variables, Y¹, Y², and Y³, can each independently have any value set forth in the claims or specification so long as a stable compound results. In certain embodiments the variables, Y¹, Y², and Y³, can have any of the following definitions.

In an embodiment Y¹, Y², and Y³ are all

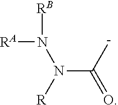

and R^{A} and R^{B} are both H and R is independently chosen at each occurrence from the 20 native amino acid side chains where any hydroxyl group in the amino acid side chain is optionally acylated with a C₂-C₆acyl group and when R is a proline the proline pyrrolidine ring is formed by R and R^{A} being joined by a —CH₂CH₂CH₂— chain. The dash (—) indicated the point of attachment of the group to one of the 3 oxygen atoms shown in Formula I.

In an embodiment Y¹ is

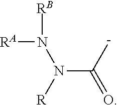

and Y² and Y³ are each hydrogen or methyl and R^{A} and R^{B} are both hydrogen.

In some embodiments R is independently chosen at each occurrence from the amino acid side chain of tyrosine optionally substituted at the tyrosine hydroxyl with Ac, a C₂-C₆acyl group, phenylalanine, leucine, isoleucine, and valine.

In some embodiments R is independently chosen at each occurrence from the amino acid side chain of tyrosine optionally substituted at the tyrosine hydroxyl with Ac, a C₂-C₆acyl group, phenylalanine, leucine, isoleucine, and valine.

Or, R can be the side chain of phenylalanine.

Formula I includes the following subformulae:

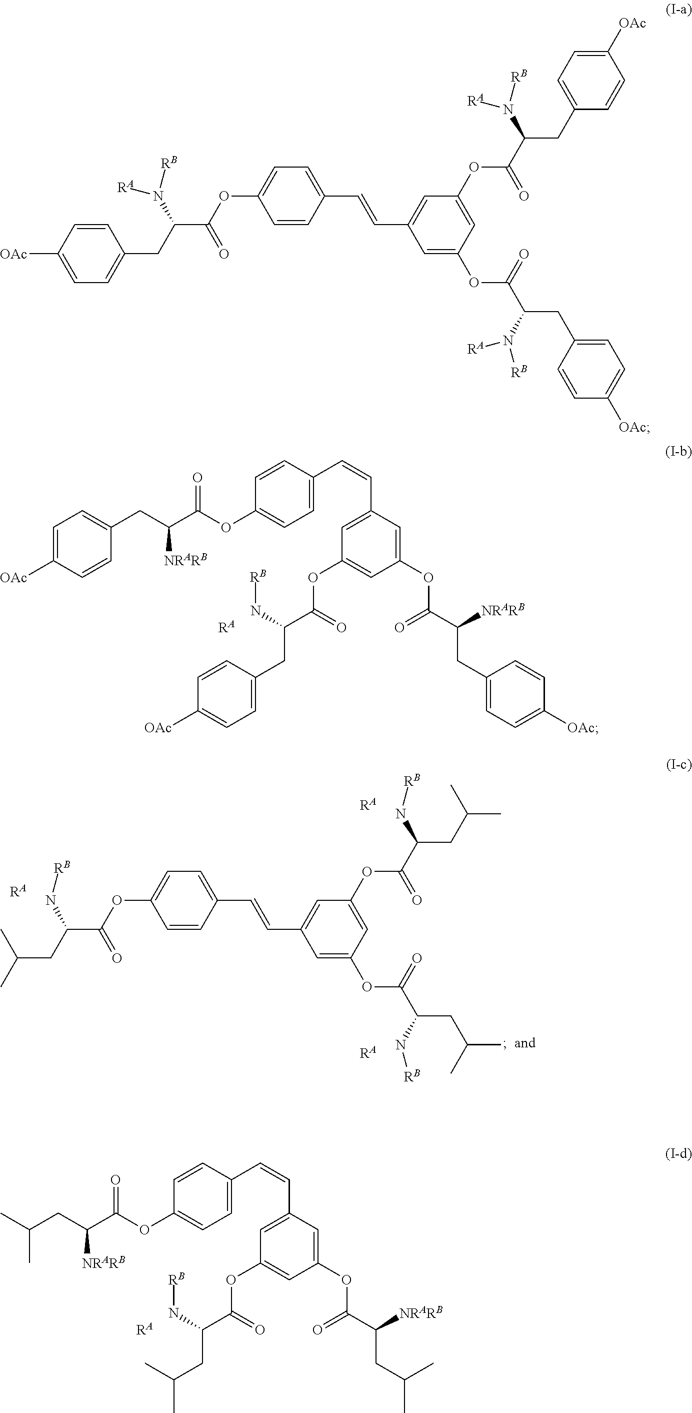

and the pharmaceutically acceptable salts of the foregoing.

This disclosure also includes the following subformulae of Formula I:

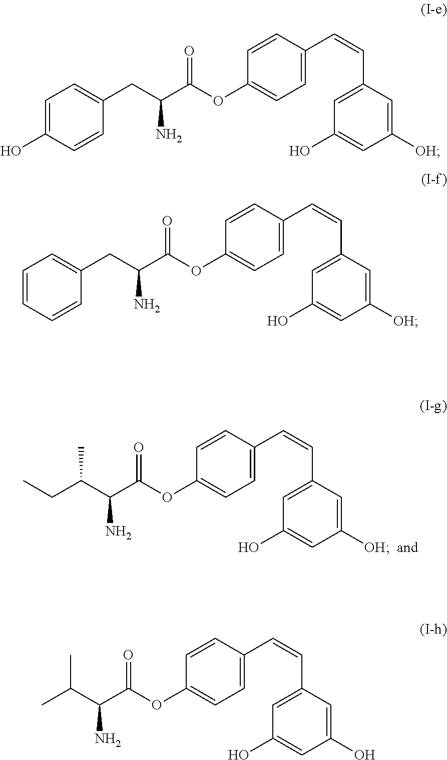

and the pharmaceutically acceptable salt of any of the foregoing.

This disclosure also includes the following exemplified compounds of Formula I and the pharmaceutically acceptable salts thereof:

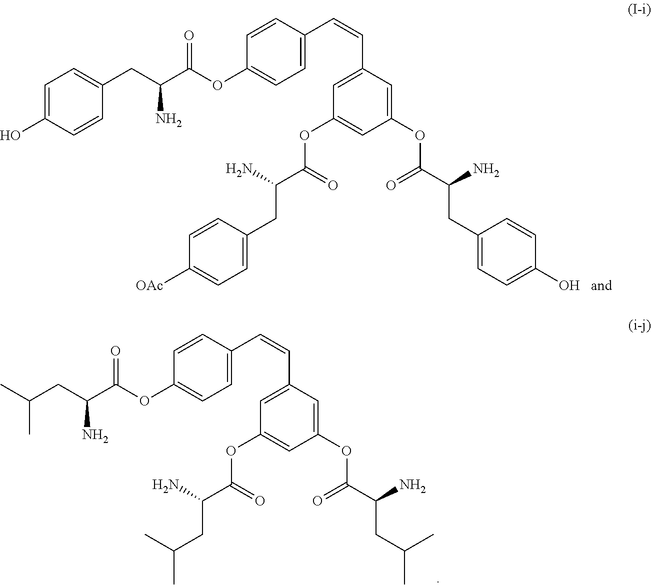

Compositions disclosed herein may include a tyrosine-mediated DNA repair inhibitor, such compounds of Formula II and the pharmaceutically acceptable salt thereof.

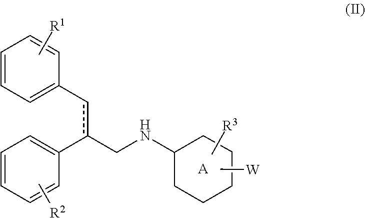

This disclosure includes compounds of the following subformulae of Formula II and the pharmaceutically acceptable salts thereof.

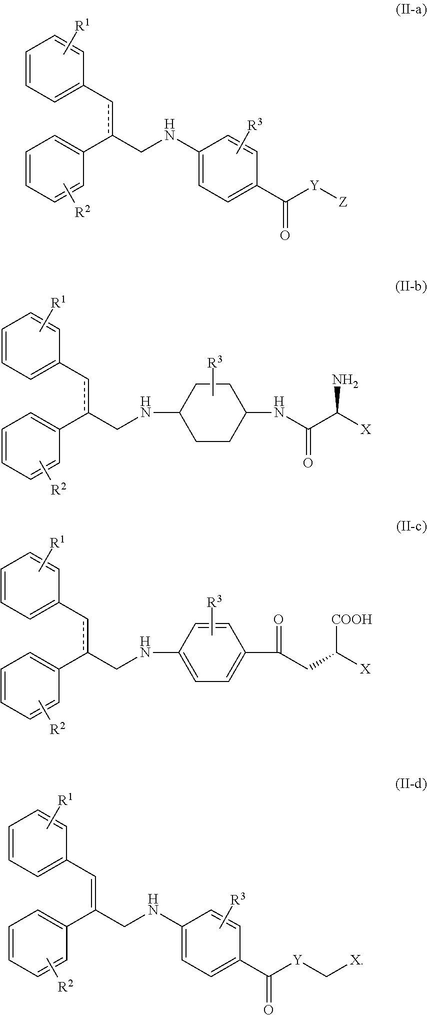

The variables, R¹, R², R², W, X, Y, Z, and the A-ring, can have the definitions set forth in the SUMMARY section, any of the definitions set forth in this specification. Any of the variable definitions can be combined so long as a stable compound results.
(i) R¹ and R² are each absent or are 1 to 2 substituents independently selected from halogen, hydroxyl, amino, cyano, C₁-C₄alkyl, C₁-C₄alkoxy, C₁-C₂haloalkyl, and C₁-C₂haloalkoxy.
(ii) R³ is absent.
(iii) Y is NH.
(iv) Y is O.
(v) X is H, —CH₂OH, or —CH(CH₃)OH.
(vi) The bond [symbol] is a double bond.
(vii) The bond [symbol] is a single bond.

The includes a compound of Formula I or salt thereof in which R¹-R³ are all absent, X is —CH(CH₃)OH and Y is NH; i.e. a compound of the formula

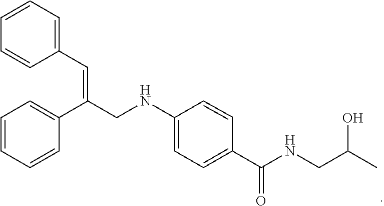 [SMILES: CC(O)CNC(=O)c1ccc(NC/C(=C/c2ccccc2)c2ccccc2)cc1]

This disclosure includes the following exemplified compound of Formula II and the pharmaceutically acceptable salts thereof.

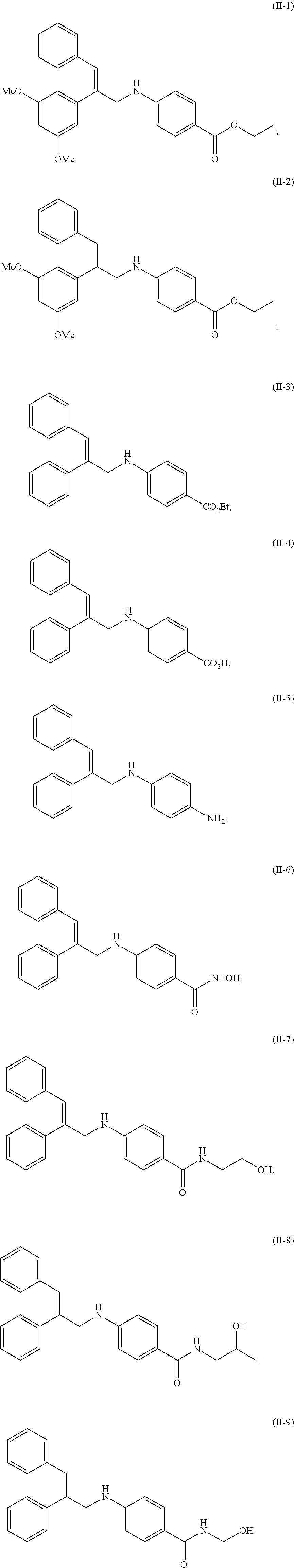

The compound II-8 may have the following stereochemistry:

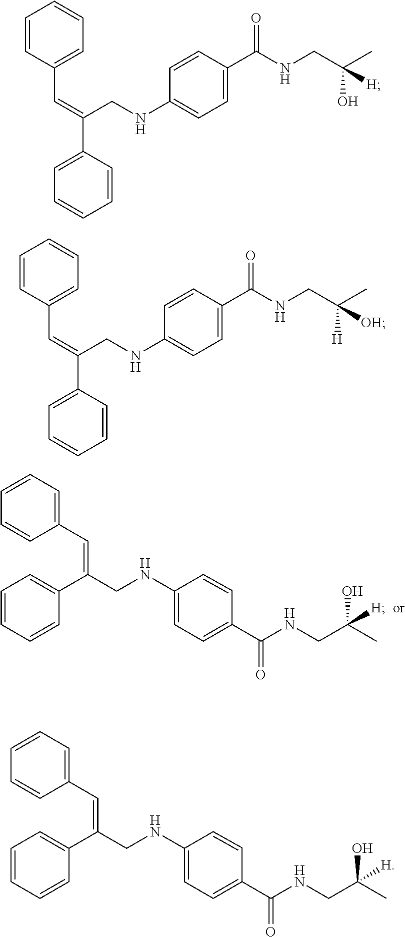

In an embodiment the compound II-8 may have the following stereochemistry:

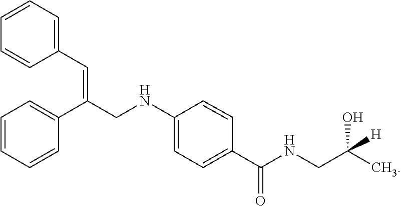 [SMILES: C[C@@H](O)CNC(=O)c1ccc(NC/C(=C/c2ccccc2)c2ccccc2)cc1]

In Formula I the variable R may be an amino acid side chain and in Formula II the variable X may be an amino acid chain. The amino acid side chain represented by variable R or X may be from an acidic amino acid. For example, the amino acid residue has a negative charge due to loss of hydrogen ion at physiological pH. Amino acids having an acidic side chain may include glutamic acid and aspartic acid.

The amino acid side chain may be from a basic amino acid residue. For example, the residue may have a positive charge due to association with hydrogen ions at physiological pH or within one or two pH units thereof. Amino acids having a basic side chain include arginine, lysine, and histidine.

The amino acid side chain may be from a hydrophobic amino acid residue. For example, the residue is not charged at physiological pH. Amino acids having a hydrophobic side chain include tyrosine, valine, isoleucine, leucine, methionine, phenylalanine, and tryptophan.

The amino acid side chain may be from a neutral/polar amino acid residue. For instance, the residue is not charged at physiological pH and the residue is not sufficiently repelled by aqueous solution so that it would seek inner positions in the conformation of a peptide or protein in which it is contained when the peptide is in an aqueous medium. Amino acids having a neutral/polar side chain include asparagine, glutamine, cysteine, histidine, serine, and threonine.

The compounds of Formula I and Formula II are resveratrol analogues. Resveratrol has two stereoisomers shown below.

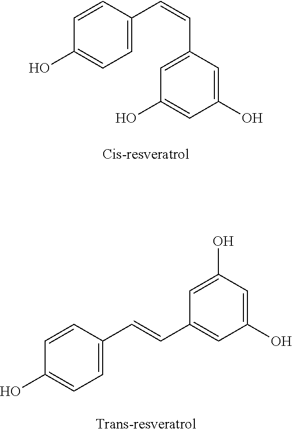 [SMILES: Oc1ccc(/C=C\c2cc(O)cc(O)c2)cc1 ; Oc1ccc(/C=C/c2cc(O)cc(O)c2)cc1]

Surprisingly, the stereo configuration of the resveratrol analogue (compound of Formula I or Formula II) can have opposing effects on tyrosine-mediated neurotoxic effects. For instance, phenylalanine and trans-RSV and analogues thereof can sustain neuronal DNA damage that exacerbates neuroinflammatory factors. In contrast, increased DNA repair mediated through elevated tyrosine levels can induce neurodegeneration by inhibiting transcription and protein synthesis. Stalled transcription has emerged as one of the hallmark of aging process. Unexpectedly, cis-RSV that increases neuronal TyrRS protein levels stimulate neuronal transcription and associated DNA damage to protect against tyrosine-mediated transcription inhibition and DNA repair and neurodegeneration. As such, cis-resveratrol and L-leucine or other amino acids that would act as tyrosine antagonists or their derivatives may serve as a neuroprotectants against tyrosine-induced neurodegeneration by activating transcription and protein synthesis. Similarly, cis-resveratrol and L-leucine or other amino acids that would act as tyrosine antagonists or their derivatives may also serve as anti-inflammatory compounds to protect against phenylalanine-induced inflammatory response.

Compositions disclosed herein may be cis-resveratrol analogues. Compound synthesized through a scheme in which D/L amino acids or their amino group derivatives connected to cis/trans-resveratrol through an ester bond such that they will be metabolized in vivo or in vitro in the cell to release/regenerate cis-resveratrol and D/L amino acids or their amino group derivatives from the administered parent compound.

In exemplified embodiments, the compounds Formula I may be of the following design:

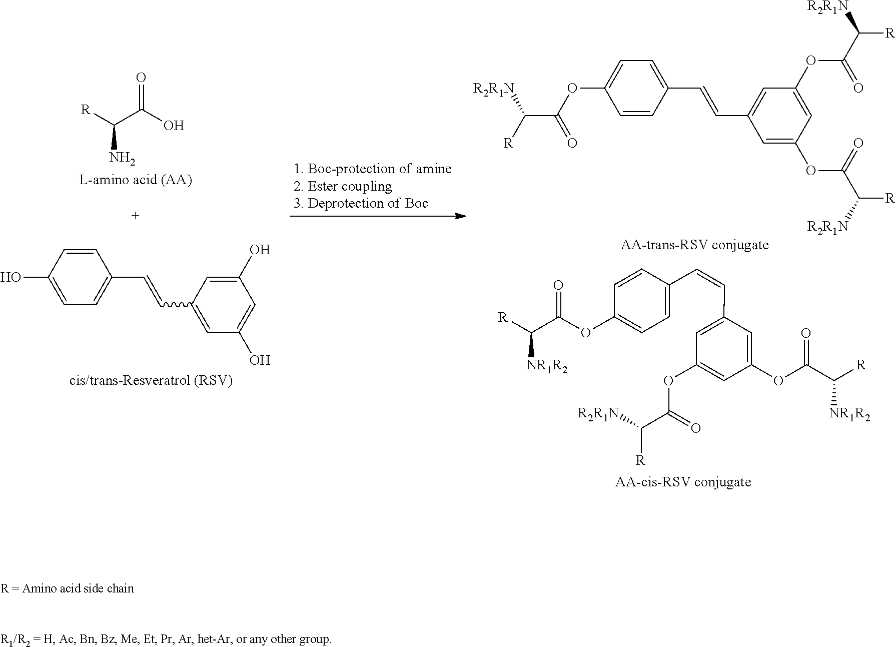

### Methods of Treatment

The disclosure includes a method of inhibiting tyrosine/phenylalanine-mediated DNA damage and repair or activating protein synthesis in a subject (e.g. a patient) comprising administering an effective amount of a compound or salt thereof of Formula I or II to the subject. The disclosure further includes a method of treating a disease or disorder associated with tyrosine/phenylalanine-mediated DNA damage and repair in a subject (e.g. a patient) comprising administering an effective amount of a compound or salt thereof of Formula I or II to the subject. A patient can be a human patient or non-human patient such as a companion animal or livestock animal. The disclosure includes use of a compound or salt thereof of Formula I or II for inhibiting tyrosine/phenylalanine-mediated DNA damage and repair or activating protein synthesis in a subject (e.g. a patient). The disclosure includes use of a compound or salt thereof of Formula I or II for treating a disease or disorder associated with tyrosine/phenylalanine-mediated DNA damage and repair in a subject (e.g. a patient). The disclosure includes methods of manufacturing a medicament of Formula I or II, or salt thereof, for use in inhibiting tyrosine/phenylalanine-mediated DNA damage and repair or activating protein synthesis in a subject (e.g. a patient) or for use in treating a disease or disorder associated with tyrosine/phenylalanine-mediated DNA damage and repair in a subject.

The disclosure also provides a method of treating a traumatic brain injury or a neurodegenerative disorder, the method comprising administering a therapeutically effective amount of (i) a compound of Formula I or II or a pharmaceutically acceptable salt of Formula I or II and (ii) a GLP-1 receptor agonist.

In one embodiment, compositions disclosed herein may be utilized in methods for inhibiting tyrosine/phenylalanine-mediated DNA damage and repair and/or activating protein synthesis. For instance, tyrosine-mediated DNA repair and inhibition of transcription and protein synthesis may be inhibited by administering a composition disclosed herein. In one embodiment, DNA repair may include DNA repair mediated by an amino acid. Amino acids mediating DNA repair may include, but are limited to, tyrosine, phenylalanine, leucine, isoleucine, valine, or a combination thereof. For instance, DNA repair may include tyrosine-mediated DNA repair. In another embodiment, DNA repair may include phenylalanine-mediated DNA repair. In another embodiment, protein synthesis inhibition may include tyrosine-mediated inhibition of protein synthesis.

In one embodiment, amino acid-mediated DNA repair may be inhibited via administration of a composition disclosed hercin in vitro or ex vivo (e.g., by contacting the cell with such compounds) or, alternatively, in vivo (e.g., administering the compound to a subject).

Increased levels of amino acids, such as tyrosine and/or phenylalanine, stimulate neuronal DNA repair and transcription inhibition. For instance, increased levels of neuronal tyrosine may inhibit protein synthesis at the clongation step depleting neuronal TyrRS. As such, following administration of cis-resveratrol, a compound of Formula I, or a compound of Formula II disclosed herein, tyrosine levels in a cell may be reduced relative to a control amount by about 10% or more, such as about 20% or more, such as about 30% or more, such as about 40% or more, such as about 50% or more, or such as about 60% or more.

This disclosure provides methods for treating and/or preventing a disorder in a subject comprising administering a therapeutically effective amount of cis-resveratrol, a compound of Formula I, or a compound of Formula II. In one embodiment, the disorder may be mediated by increased tyrosine levels. In another embodiment, the disorder may be aging.

The disorder can be an age-associated neurocognitive disorder. The age-associated neurocognitive disorders include but are not limited to, insomnia, hypersomnia, frontotemporal dementia, vascular dementia, Alzheimer's disease, Parkinson's disease, Lewy body dementia, Wernicke-Korsakoff syndrome, multiple sclerosis, amyotrophic lateral sclerosis, multiple sclerosis, Huntington's disease, epilepsy and seizures, learning disabilities, neuromuscular disorders, Cockayne syndrome, cerebral palsy, dystonia, spinocerebellar ataxia with axonal neuropathy-1 (SCAN1), Angelman Syndrome, COVID-19-related neurocognitive problems, chemotherapy-associated neurocognitive problems including ‘chemo brain’, autism spectrum disorder (ASD), delirium, mild-cognitive impairment, traumatic brain injury, phenylketonuria, or tyrosinemia. In one embodiment, the age-associated neurocognitive disorder is Alzheimer's disease.

The disorder can be an age-associated metabolic disorder. Age-associated metabolic disorders include, but are not limited to, heart failure, cardiovascular disease, autoimmune-related disorders, myocardial ischemia reperfusion injury, hypertension, stroke, septic encephalopathy, diabetes, obesity, sepsis, Systemic Lupus Erythematosus, metabolic syndrome, and inflammation.

In an embodiment the method is a method of treating a neurodegenerative disease in a patient and the neurodegenerative disease is neurodegenerative disease and the neurogenerative disease is Alzheimer's dementia, frontal cortex dementia, frontotemporal dementia (FTD), Mixed Dementia, Huntington's disease, vascular dementia, Wernicke-Korsakoff syndrome, Lewy body dementia, Multiple Sclerosis or Parkinson's disease

In an embodiment the method is a method of treating a traumatic brain injury in a patient. Because both agents in the combination are very safe and suitable for continued use, this disclosure also includes prophylactic treatment of a person at high risk for a traumatic brain injury, for example prophylactic treatment of boxers, rugby players, football players, or American football players. Prophylactic treatment can decrease the severity of symptoms of a traumatic brain injury. Methods of treating a traumatic brain injury include treatment immediately following the injury or within 1, 2, 3, 4, 5, 6, 7, 8, 9, 10, 11, 12, 18, or 24 hours of the traumatic brain injury. Symptoms of traumatic brain injury include headache, nausea or vomiting, fatigue or drowsiness, dizziness, balance issues, sensitivity to light or noise, blurred or double vision, seizures, confusion or disorientation, difficulty concentrating or focusing, short and long term memory problems, reduced cognitive function, mood swings, irritability, agitation, anxiety, depression, changes in sleep patterns, loss of consciousness, difficulty with coordination, and changes in taste or smell. Methods of treatment includes administering an amount of the disclosed combination of (i) a compound of Formula I or II or a pharmaceutically acceptable salt of Formula I or II and (ii) a GLP-1 receptor agonist effective to reduce one or more of these symptoms of traumatic brain injury, or if given prophylactically, sufficient to decrease the probability of developing one or more of these symptoms or decreasing the severity of symptoms should a TBI occur.

In an embodiment methods of treatment include administering a compound or salt of Formula I and the therapeutically effective amount of the compound of Formula I of II is 1 mg to 500 mg daily, 5 mg to 3500 mg administered weekly, or 20 mg to 15 g administered monthly; and

The GLP-1 receptor agonist can be liraglutide, semaglutide, dulaglutide, exenatide, lixisenatide, tirzepatide, danuglipron (CAS Reg. No. 2230198-02-2), and orforglipron (CAS Reg. No. 2212020-52-3). The therapeutically effective amount of the GLP-1 receptor agonist can be 0.1 to 2 mg of the GLP-1 receptor agonist administered daily, 0.1 to 3 mg of the GLP-1 receptor agonist administered daily, 0.1 to 1 mg of the GLP-1 receptor agonist administered daily, 1 to 15 mg, 1 to 10 mg, or 2 to 10 mg of the GLP-1 receptor agonist administered every week, 2 to 30 mg, 1 to 50 mg, or 5 to 30 mg of the GLP-1 receptor agonist administered twice monthly, or 4 to 60 mg, 5 to 50 mg, or 10 to 50 mg of the GLP-1 receptor agonist administered monthly.

In an embodiment the (i) the compound of Formula I of II or salt of Formula I or II and (ii) the GLP-1 receptor agonist or salt thereof are administered together, either in the same dosage form or in separate dosage forms but at the same time (within one hour).

In an embodiment the (i) the compound of Formula I or salt thereof and (ii) the GLP-1 receptor agonist or salt thereof are administered sequentially.

In an embodiment treating a TMI includes (i) the one of the compound of Formula I or II or salt thereof, and (ii) the GLP-1 receptor agonist are both administered to the patient within 1 hour, 2 hours, 6 hours, 12 hours, or 24 hours of the patient incurring a traumatic brain injury.

The therapeutically effective amount of the composition can vary based on factors such as the disorder stage, age, sex, and weight of the individual, and the ability of the compound to clicit a desired response in the subject. Further, compounds disclosed herein can be administered to a subject at one time or over a series of treatments and may be administered to the subject at any time.

Interestingly, in accordance with the present disclosure, nanomolar concentration of compounds disclosed herein may exacerbate undesirable neurotoxic effects that leads to the depletion of neuronal TyrRS and increases levels of neuroinflammatory factors. As such, compositions disclosed herein that are in the trans configuration may be introduced to a cell at a concentration less than 25 nM. As such, compounds disclosed herein may be introduced to a cell at a concentration from about 1 micromolar (μM) to about 100 μM, such as from about 2 μM to about 95 μM, such as from about 10 μM to about 85 μM, such as from about 20 μM to about 75 μM, such as from about 35 μM to about 50 μM, or any range therebetween. As expected, the dosage will be dependent on the condition, size, and age of the subject.

Compounds disclosed herein may be administered, as appropriate or indicated, in a single dose as a bolus or by continuous infusion, or as multiple doses by bolus or by continuous infusion. Multiple doses may be administered, for example, multiple times per day, once daily, multiple times per week, every 2, 3, 4, 5, 6 or 7 days, weekly, every 2, 3, 4, 5 or 6 weeks, or monthly. However, other dosage regimens may be useful. The progress of this therapy is easily monitored by conventional techniques.

In an embodiment methods of treatment include administering cis-resveratrol, a compound of Formula I, or a compound of Formula II, or a pharmaceutically acceptable salt of any of the foregoing and the therapeutically effective amount of cis-resveratrol, the compound of Formula I, or the compound of Formula II is 0.1 mg to 2000 mg daily, 0.5 mg to 15 g administered weekly, or 20 mg to 500 g administered monthly. The amount of cis-resveratrol, the compound of Formula I, or the compound of Formula II administered daily can be 0.1 mg to 2000 mg daily, 0.1 mg to 1500 mg daily, comprising 0.1 to 1000 mg daily, 0.1 to 900 mg daily, 0.1 to 700 mg daily, 0.1 to 600 mg daily, 0.1 to 500 mg daily, 0.1 to 400 mg daily, 0.1 to 300 mg daily, 0.1 to 200 mg daily, 0.1 to 100 mg daily, 0.1 to 50 mg daily, 0.1 to 25 mg daily, 0.1 to 10 mg daily, 0.1 to 5 mg daily, 1.0 to 1000 mg daily, 1.0 to 900 mg daily, 1.0 to 700 mg daily, 1.0 to 600 mg daily, 1.0 to 500 mg daily, 1.0 to 400 mg daily, 1.0 to 300 mg daily, 1.0 to 200 mg daily, 1.0 to 100 mg daily, 1.0 to 50 mg daily, 1.0 to 25 mg daily, 1.0 to 10 mg daily, 1.0 to 5 mg daily, 10 to 1000 mg daily, 10 to 900 mg daily, 10 to 700 mg daily, 10 to 600 mg daily, 10 to 500 mg daily, 10 to 400 mg daily, 10 to 300 mg daily, 10 to 200 mg daily, 10 to 100 mg daily, 10 to 50 mg daily, 100 to 1000 mg daily, 100 to 900 mg daily, 100 to 700 mg daily, 100 to 600 mg daily, or 100 to 500 mg daily.

When cis-resveratrol, the compound of Formula I, or the compound of Formula II is administered daily, it can be administered 1, 2, 3, or 4 or more times daily. Once or twice daily administration is preferred.

Methods of treatment include administering a sufficient amount of cis-resveratrol, a compound of Formula I, or a compound of Formula II or salt of any of the foregoing to provide a plasma or blood Cₘₐₓ of 100 ng/ml, 90 ng/ml, 80 ng/ml, 70 ng/ml, 60 ng/ml, 50 ng/ml, 40 ng/ml, 30 ng/ml, 20 ng/ml, or 10 ng/ml.

### Pharmaceutical Compositions and Pharmaceutical Dosage Forms

In one embodiment, a pharmaceutical composition comprising cis-resveratrol, a compound of Formula I, or a compound of Formula II, or pharmaceutically acceptable salt of any of the foregoing, together with at least one pharmaceutically compatible excipient can be delivered to the targeted cells or tissue via a pharmaceutically acceptable delivery system.

Pharmaceutical excipients include carriers, buffering agents, coloring agents, diluents, disintegrants, emulsifiers, flavorants, glidants, lubricants, preservatives, stabilizers, surfactants, tableting agents, and wetting agents. In some instances definitions of these classes of excipients overlap.

Pharmaceutically acceptable carriers include, but are not limited to, saline, buffered saline, glucose in saline, etc. Solid supports, liposomes, nanoparticles, microparticles, nanospheres or microspheres may also be used as carriers for administration of a compound disclosed herein. As used herein, the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. Examples of suitable aqueous and nonaqueous carriers, diluents, solvents or vehicles include, but are not limited to, water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. The use of such media and agents for pharmaceutically active substances is well-known in the art. Supplementary agents can also be incorporated into the compositions.

It can be advantageous to formulate oral or parenteral compositions in dosage unit form for case of administration and uniformity of dosage. Dosage unit form as used herein includes physically discrete units suited as unitary dosages for the subject to be treated; each unit may contain a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

Pharmaceutical compositions for parenteral, intradermal, or subcutaneous injection can include pharmaceutically acceptable sterile aqueous or nonaqueous solutions, dispersions, suspensions or emulsions, as well as sterile powders for reconstitution into sterile injectable solutions or dispersions just prior to use.

A composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions, and by the use of surfactants. A composition may also contain adjuvants such as preservatives, wetting agents, emulsifying agents, and dispersing agents. It may also be desirable to include isotonic agents such as sugars, sodium chloride and the like.

For intravenous administration, suitable carriers include, without limitation, physiological saline, bacteriostatic water, Cremophor EL™ (BASF™, Parsippany, N.J.) or phosphate buffered saline (PBS). In all cases, an injectable composition should be sterile and should be fluid to the extent that easy syringeability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents and/or adjuvant materials can be included as part of an orally ingestible composition. The tablets, pills, capsules, troches and the like can contain any of the following ingredients or compounds of a similar nature: a binder such as microcrystalline cellulose, gum tragacanth, or gelatin; an excipient such as starch or lactose; a disintegrating agent such as alginic acid, Primogel®, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water, petroleum, oils of animal or plant origin (e.g., peanut oil, mineral oil, soybean oil, or sesame oil), or synthetic oils may be added. A liquid form may further contain physiological saline solution, dextrose or other saccharide solution, or glycols such as ethylene glycol, propylene glycol, or polyethylene glycol. When administered in liquid form, a composition can contain from about 0.5 to 90% by weight of the cis-resveratrol derivative.

For administration by inhalation, a compound, e.g., a cis-resveratrol derivative may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, a pharmaceutical composition can be formulated for sustained or controlled release of the compound (e.g., cis-resveratrol derivative). Biodegradable, biocompatible polymers, such as ethylene vinyl acetate, polyanhydrides, polyglycolic acid, collagen, polyorthoesters, and polylactic acid can be used. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art.

The pharmaceutical composition can be formulated for oral administration. The oral dosage form can comprise from 0.1 to 99% by weight (wt %) of cis-resveratrol, the compound of Formula I, or the compound Formula II, or a pharmaceutically acceptable salt of any of the foregoing. Some embodiments contain from 1 to 99%, 1 to 90%, 1 to 80%, 1 to 70%, 1 to 60%, 1 to 50%, 1 to 40%, 1 to 30%, 1 to 25%, 1 to 20%, 5 to 99%, 5 to 90%, 5 to 80%, 5 to 70%, 5 to 60%, 5 to 50%, 5 to 40%, 5 to 30%, 5 to 25%, 5 to 20%, 10 to 99%, 10 to 90%, 10 to 80%, 10 to 70%, 10 to 60%, 10 to 50%, 10 to 40%, 10 to 30%, 20 to 99%, 20 to 90%, 20 to 80%, 20 to 70%, 20 to 60%, 20 to 50%, or 20 to 40% by weight (wt %) of the compound or salt thereof of cis-resveratrol, Formula I, or Formula II.

In an embodiment the dosage form comprises of cis-resveratrol, a compound of Formula I, or a compound of Formula II, or a pharmaceutically acceptaly salt of any of the foregoing, and is an immediate or extended release dosage from comprising 0.1 to 1000 mg, 0.1 to 900 mg, 0.1 to 700 mg, 0.1 to 600 mg, 0.1 to 500 mg, 0.1 to 400 mg, 0.1 to 300 mg, 0.1 to 200 mg, 0.1 to 100 mg, 0.1 to 50 mg, 0.1 to 25 mg, 0.1 to 10 mg, 0.1 to 5 mg, 1.0 to 1000 mg, 1.0 to 900 mg, 1.0 to 700 mg, 1.0 to 600 mg, 1.0 to 500 mg, 1.0 to 400 mg, 1.0 to 300 mg, 1.0 to 200 mg, 1.0 to 100 mg, 1.0 to 50 mg, 1.0 to 25 mg, 1.0 to 10 mg, 1.0 to 5 mg, 10 to 1000 mg, 10 to 900 mg, 10 to 700 mg, 10 to 600 mg, 10 to 500 mg, 10 to 400 mg, 10 to 300 mg, 10 to 200 mg, 10 to 100 mg, 10 to 50 mg, 100 to 1000 mg, 100 to 900 mg, 100 to 700 mg, 100 to 600 mg, or 100 to 500 mg of the compound of Formula I or Formula II or salt thereof per unit dosage form.

In an embodiment the dosage form comprises cis-resveratrol, a compound of Formula I, or a compound of Formula II, or a pharmaceutically acceptable salt of any of the foregoing, and is an extended release dosage form formulated for once weekly to once monthly administration that provides a plasma Cₘₐₓ of 0.1 to 100 nM cis-resveratrol, of the compound of Formula I, or Formula II to the patient. The form can be an extended release dosage form.

The disclosure provides combination dosage forms comprising (i) cis-resveratrol, the compound of Formula I, or the compound of Formula II, or a pharmaceutically acceptable salt of any of the foregoing and (ii) the GLP-1 receptor agonist or salt thereof.

In an embodiment the dosage form comprises cis-resveratrol, a compound of Formula I, a compound of Formula II, or a pharmaceutically acceptable salt of any of the foregoing.

In an embodiment the GLP-1 receptor agonist is exenatide or a pharmaceutically acceptable salt thereof.

In an embodiment the dosage form comprises cis-resveratrol or salt thereof, a compound or salt of Formula I or a compound or salt of Formula II and is an extended release dosage form formulated for once weekly to once monthly administration that provides a plasma Cₘₐₓ of 0.1 to 100 nM of the compound of Formula I or II to the patient. The form can be an extended release dosage form.

In an embodiment the dosage form comprises cis-resveratrol, a compound of Formula I, or a compound of Formula II, or a pharmaceutically acceptable salt thereof and is an extended release dosage form formulated for once weekly to once monthly administration that provides a daily dosage of 0.1 to 500 mg cis-resveratrol, of the compound of Formula I, or the compound of Formula II, or a pharmaceutically acceptable salt of any of the foregoing to the patient. The form can be an extended release dosage form. The form can be an extended release dosage form.

In an embodiment the dosage form is a dosage form for weekly administration comprising 1 to 15 mg of the GLP-1 receptor agonist, a dosage for every other week administration comprising 2 to 30 mg of the GLP-1 receptor agonist, or a monthly dosage form for monthly administration comprising 4 to 60 mg of the GLP-1 receptor agonist.

The present disclosure may be better understood with reference to the following examples.

### EXAMPLES

ABBREVIATIONS

| | |
|---|---|
| AcOH | Acetyl alcohol |
| DCE | 1,2-Dichloroethane |
| DIBAL | Diisobutylaluminum hydride |
| Et₃N | Triethyl amine |
| EtOH | Ethanol |
| Na(OAc)₃BH | Sodium triacetoxyhydroborate |
| OTBS | O-(tert-Butyldimethylsilyl)hydroxylamine |
| rt or RT | Room temperature |
| TBAF | Tetra-n-butylammonium fluoride |
| THF | Tetrahydrofuran |

### Materials and Methods

### Primary Neuronal Culture

Primary cortical neurons were harvested from 18-day-old Sprague Dawley rat pups using Hibernate E (BrainBits) and dissociated with the Neural Tissue Dissociation kit (Miltenyi Biotec). Briefly, the cortices were minced cortices and incubated in a pre-heated enzyme mix at 37° C. for 15 minutes. The tissues were then strained using a 40 μm cell strainer, washed, and centrifuged. The neurons were then cultured on tissue culture plates coated with 50 μg/ml poly-D-Lysine (Sigma Aldrich). The culture medium consisted of NBActive-1 medium (BrainBits) supplemented with 100 U/ml of Penicillin-Streptomycin (Life Technologies), 2 mM L-Glutamine (Life Technologies), and 1× N21 supplement (R&D Systems) was used as culture medium. Control (non-targeting), TyrRS, and PARP1 siRNAs were obtained from Invitrogen (#AM4635, s443, and s130207, respectively). 5 DIV neurons were transfected with 75 nM control or TyrRS siRNA using Dharmafect 3 Transfection Reagent. A second transfection was done two days later using 75 nM of TyrRS siRNA, followed by cell collection or assays after another 48 hr. For PARP1 siRNA, 7 DIV neurons were transfected with either 75 nM control or PARP1 siRNA.

### Ribosome Profiling

Briefly, ribosome profiling for cortical neurons were washed twice in ice-cold PBS supplemented with 100 μg/ml cycloheximide. Neurons were collected with a scraper in polysome lysis buffer [20 mM Tris pH 7.5, 150 mM NaCl, 5 mM MgCl₂, 100 μg/ml cycloheximide, 1 mM DTT, protease inhibitor cocktail (Roche), and 8% glycerol]. After scraping, the lysates were supplemented with Triton X100 to a final concentration of 1% and chilled on ice for 10 min followed by trituration (10 times) using a 23-gauge syringe. Samples were chilled on ice for 10 min and then cleared by centrifugation at 16,100 g for 10 min. For ribosome profiling, samples were loaded onto 6-ml 10% to 50% sucrose density gradients that were prepared w/v in the polysome lysis buffer. Gradients were centrifuged for 2 hours 45 min at 36,000 revolutions per minute (rpm) at 4° C. in a SW41 Ti swing-out rotor. Following the centrifugation, 100 μL fractions were collected, followed by measurement of absorbance at 254 nm to obtain the ribosome profiles. All the procedures were done using ice-cold conditions and with RNase-free reagents.

### RNA Extraction and Library Preparation

RNA and library preparation, and post-processing of the raw data were performed by at a COBRE Functional genomics Core. RNAs were extracted Zymo Quick-RNA MicroPrep Kit as per manufacturer recommendations and RNA clean by Zymo RNA Clean and Concentrator Kit (Zymo Research, Irvine, CA, USA). RNA quality was evaluated on RNA-1000 chip using Bioanalyzer (Agilent, Santa Clara, CA, USA). RNA libraries were prepared using established protocol with NEBNExt Ultra II Directional Library Prep Kit (NEB, Lynn, MA). Each library was made with one of the TruSeq barcode index sequences and the Illumina sequencing done by Novogene (Sacramento, CA) with Illumina HiSeq PE150 (150bp, pair-ended). Sequences were aligned to the Human genome GRCh38.77 (GCA_000001405.15, ensemble release-77) using STAR v2.4. Samtools (v1.2) were used to convert aligned sam files to bam files and reads were counted using the feature Counts function of the Subreads package using Homo_sapiens.GRCh38.77.gtf annotation file. Only reads that were mapped uniquely to the genome were used for gene expression analysis. Differential expression analysis was performed in R using the DeSeq2 package, followed by gene ontology using ShinyGO.

### EU Incorporation Assay

The Click-iT® RNA Alexa Fluor® 594 Imaging Kit (Invitrogen, C10330) was used for gene EU (5-ethynyl Uridine) incorporation assay. Briefly, the stocks solutions were prepared as per manufacturer's instructions. After the treatment, 2× working solution from 100 mM stock of EU was added to well, for a final concentration of 1 mM in each well followed by incubation for 1 hr at 37° C. The media was then removed, and the wells were washed twice with PBS before fixing them using 2% paraformaldehyde for 15 min at room temperature. The fixed cells were then washed three times with PBS and were then blocked using 5% BSA with 0.1% Tween 20 for 30 min. Freshly prepared 1× Click IT solution and Click IT cocktail were added immediately after removing the blocking solution and the cells were then allowed to incubate for 30 min in dark. After 30 min, the click IT cocktail solution was removed, and the cells were washed with Click IT rinse buffer followed by three washes with PBS. Finally, the coverslips were dried and then mounted using DAPI (4′,6-diamidino-2-phenylindole)-supplemented mounting medium, Prolong Gold Antifade (Invitrogen) and imaged with Leica DMI6000 epifluorescent microscope using oil immersion 63×/NA 1.4 objective. The quantification for total protein levels in neurons was done using ImageJ (Version 1.53c), and imaging parameters were matched for exposure, gain, and offset.

### Western Blotting

Cultured primary rat cortical neurons (DIV 9/10) were prepared for analysis by washing with cold 1×PBS and lysing in cell lysis buffer. The lysates were then centrifuged at 15,000 g for 15 min at 4° C. to separate the chromatin-bound and soluble fractions and equal amounts of protein were loaded onto a 4% to 12% gradient gel (NuPAGE-Invitrogen) for electrophoresis. The protein was transferred to a 0.2 μm NC membrane and the membrane was blocked with 5% non-fat milk in TBST. Primary antibodies were applied to the membrane and incubated overnight at 4° C., followed by incubation with secondary antibodies for 1 hour at room temperature. The immobilon ECL Ultra Western HRP Substrate was used to detect the proteins, and the luminescent image analyzer (ChemiDoc Imaging System, Bio-Rad) was used for quantification. The western blots were quantified using ImageJ software (Version 1.53t).

List of antibodies used for western blotting

| Antibody | Company | Catalog No. | Dilution |
|---|---|---|---|
| ARH3 | Proteintech | 16504-1-AP | 1:500 |
| α-Tubulin | Proteintech | 66031-1-Ig | 1:2000 |
| c-FOS | Cell Signaling Technology | 2250 | 1:1000 |
| DDB1 | US Biological | 522188 | 1:500 |
| | Life Sciences | | |
| eEF2 | Cell Signaling Technology | 2332 | 1:1000 |
| Fen1 | Proteintech | 14768-1-AP | 1:1000 |
| GAPDH | Cell Signaling Technology | 2118 | 1:2000 |
| H3 | Proteintech | 17168-1-AP | 1:1000 |
| H3-S10-ADP-Ribose | Bio-RAD | HCA357 | 1:1000 |
| MRE11 | Cell Signaling Technology | 4847 | 1:200 |
| NuMA | Invitrogen | PA1-32451 | 1:500 |
| p65/RelA | Cell Signaling Technology | 8242 | 1:500 |
| PARP1 | Proteintech | 66520-1-Ig | 1:1000 |
| PheRSα | Proteintech | 18121-1-AP | 1:1000 |
| PheRSβ | Proteintech | 16341-1-AP | 1:1000 |
| Phospho-eEF2 (Thr56) | Cell Signaling Technology | 2331 | 1:1000 |
| Phospho-Histone H3 | Cell Signaling Technology | 53348S | 1:1000 |
| (Ser10) | | | |
| Poly (ADP-Ribose) | Abcam | ab14459 | 1:1000 |
| Polymer | | | |
| Puromycin | Millipore | MABE343 | 1:2000 |
| RNF4 | Sigma | SAB1100322 | 1:500 |
| STAT3 | Cell Signaling Technology | 9139 | 1:1000 |
| TrpRS | Proteintech | 16081-1-AP | 1:1000 |
| TOP1 | Novus Biologicals | NBP1-90365 | 1:500 |
| TOP2β | Novus Biologicals | NBP1-89527 | 1:1000 |
| TyrRS | Abcam | ab50961 | 1:1000 |
| UBE3A | Cell Signaling Technology | 7526 | 1:1000 |
| VCP | Proteintech | 10736-1-AP | 1:1000 |
| β-Tubulin | Cell Signaling Technology | 2128 | 1:2000 |

### Comet Assay

The cells were harvested with a cell scraper using chilled PBS and counted. The comet assay (Trevigen Inc, Gaithersburg, MD) was performed according to manufacturer's protocol using alkaline conditions). Briefly, after electrophoresis, the slides were washed twice in deionized water for 5 min and immersed in 70% ethanol for 5 min. Subsequently, the slides were dried at 37° C. for 30 min. DNA staining was done using SYBR Gold dye (Fisher Scientific, 1:10000 in Tris-EDTA buffer, pH 7.5) for 20 min in the dark at room temperature and then imaged using an epifluorescent microscope at 10× magnification. The images were quantified and scored for comet parameters such as tail length using the Tritek CometScore™ Freeware v1.5 image analysis software.

### DNA Fiber Analysis

Cultured cortical neurons (DIV 9/10) were harvested using chilled PBS and counted. Briefly, cells were isolated by trypsinization, embedded in agarose plugs, and subjected to proteinase K (0.5% SDS, 0.1 M EDTA, 1 mg/ml Proteinase K) digestion at 50° C. for 16 hr. Plugs were dissolved with agarose (Fisher [NEB], 50-811-726) for 16 hr. Molecular combing was performed using the FiberComb® Molecular Combing System (Genomic Vision) with a constant stretching factor of 2 kb/um using vinylsilane coverslips (20×20 mm; Genomic Vision), according to the manufacturer's instructions. Combed coverslips were incubated at 60° C. for 2 hr in a pre-warmed hybridization oven to minimize photo-breaking, followed by denaturation of the DNA fibers (0.5 M NaOH+1 M NaCl) for 8 min. The coverslips were then washed with PBS, followed by serial ethanol dehydration (70%-100%). Following two 1×PBS washes, the coverslips were blocked in 3% BSA/1×PBS for 30 min followed by incubation with α-BrdU (for CldU) (Accurate Chemical, OBT0030) (1:100) and ssDNA antibody (Millipore MAB3034) (1:100), for 2 hr at 37° C. After three PBST washes, secondary antibody incubation was done using a-mouse AlexaFluor 594 and α-rat AlexaFluor 488 (1:500) for 1 hr at 37° C. Coverslips were washed three times with 1×PBST, dehydrated and mounted on slides with mounting media. The stained DNA fibers were visualized using a fluorescence microscope (EVOS FL, ThermoFisher Scientific). Analysis was performed in ImageJ by counting the total ssDNA (red) and the CldU labeled fibers (green). For each treatment condition, 300 fibers were counted, and the average ratio of CldU incorporation for ssDNA fibers per condition was used for final representation.

### Immunofluorescence (IF)

Cultured cortical neurons at DIV 9-10 were fixed in 4% formaldehyde for 15 minutes, permeabilized, and blocked with 5% BSA (PBS) and 0.1% Tween20 for 30 minutes at room temperature. Primary antibodies were added and incubated overnight at 4° C., followed by secondary antibody incubation for 1 hour at room temperature. Alexa Fluor 647 (anti-chicken), Alexa Fluor 555 (anti-mouse), and Alexa Fluor 488 (anti-rabbit) from Invitrogen were used as secondary antibodies at a dilution of 1:1000. Coverslips were mounted with DAPI-supplemented mounting medium, Prolong Gold Antifade (Invitrogen), and imaged with a Leica DMI6000 epifluorescent microscope using an oil immersion 63×/NA 1.4 objective. The quantification of total protein levels in neurons was performed using ImageJ (Version 1.53c), with imaging parameters matched for exposure, gain, and offset. Neuronal γ-H2AX foci were calculated.

List of antibodies used for IF

| Antibody | Company | Catalog No. | Dilution |
|---|---|---|---|
| CldU | Cell Signaling Technology | 5292S | 1:500 |
| MAP2 | Abcam | Ab5392 | 1:500 |
| phospho-histone | Cell Signaling Technology | 9178 | 1:400 |
| H2AX (Ser139) | | | |
| TyrRS | Novus Biologicals | NBP1-32551 | 1:200 |
| Phospho-Histone | Cell Signaling Technology | 53348S | 1:600 |
| H3 (Ser10) | | | |

### Pharmacological Treatment

All drugs/inhibitors stock solutions (1000×) were prepared in DMSO or ethanol and diluted in culture media to their final concentration. The various compounds used for treatments and their stock concentrations are listed below:

| | | Stock | | Final | | |
|---|---|---|---|---|---|---|
| Compound | Catalog | Concentration | | Concentration | | Solvent |
| L-Tyr | 194759, MP Biomedicals | 100 | mM | 0.1-0.5 | mM | PBS |
| Aβ42 | A9810, Sigma Aldrich | 100 μM, | | 50 nM, | | PBS |
| | | 100 nM | | 50 pM | | |
| Aβ40 | A1075, Sigma Aldrich | 100 μM, | | 50 nM, | | PBS |
| | | 100 nM | | 50 pM | | |
| AB42-1 | SCP0048, Sigma Aldrich | 100 | μM | 100 | nM | PBS |
| Etoposide | 28435, Chem Implex | 100 | mM | 10 | μM | DMSO |
| Camptothecin | 276721000, Acros Organics | 100 | mM | 10 | μM | DMSO |
| Thiorphan | T6031, Sigma Aldrich | 100 | μM | 100 | nM | Ethanol |
| cis-RSV | 10004235, Cayman Chemicals | 100 | mM | 50 | μM | Ethanol |
| trans-RSV | 34092, Millipore-Sigma | 100 | mM | 50 | μM | Ethanol |
| Verubecestat | HY-16759, MedChemExpress | 100 | μM | 100 | nM | DMSO |
| Emetin | 324693, Millipore | 10 | mM | 10 | μM | PBS |
| Anisomycin | A9789, Sigma | 10 | mM | 10 | μM | DMSO |
| NMDA | 0114, Tocris | 50 | mM | 50 | μM | PBS |

### Neurite Degeneration Index

Briefly, samples were imaged using ImageXpress Micro 4 at a magnification of 10× to capture the entire field of interest. The samples were stained with MAP2 (Alexa fluor 647) for neurites and DAPI for the nucleus. The analysis of neurite degeneration was done using ImageJ. The images are then processed using ImageJ software to convert pixel intensity of the neurite staining to black and all other regions to white. The particle analyzer module of ImageJ is used to calculate the percentage of fragmented neurite area (size=3-10 μm²) to the intact neurite area (size>25 μm²). The degeneration index (DI) is calculated as the ratio of the fragmented neurite area to the intact neurite area. This DI was used as a measure of the health of the neurites in the sample, with higher values indicating greater degeneration.

### Cell Viability Assays

Rat cortical neurons (DIV 9/11) were seeded at a density of 20,000 cells/well in 96-well plates. Cultured rat cortical neurons were incubated with MTT (0.5 mg/mL) for 2 hr. The MTT assay is a colorimetric assay used to determine cell viability by measuring the reduction of the soluble tetrazolium salt, MTT, to an insoluble formazan product that can be quantified spectrophotometrically. The assay is based on the principle that living cells have active mitochondria that can reduce MTT to formazan, while dead or dying cells cannot perform this reduction. The absorbance measurement at 570 nm is proportional to the number of live cells, as it reflects the amount of formazan produced by the reduction of MTT. The results are presented as a percentage of control (wells incubated with the vehicle).

### Statistical Analysis

The data was analyzed for statistical significance and differences between groups were determined. Depending on the number of groups being compared, either one-way ANOVA with multiple comparisons without correction or two-way ANOVA with multiple comparisons without correction were used. When comparing two groups, either paired or unpaired t-tests were used. The data analysis was performed using GraphPad statistical analysis software.

### Example 1. Synthesis of Ethyl (E)-4-((2-(3,5-Dimethoxyphenyl)-3-Phenylallyl)Amino)Benzoate

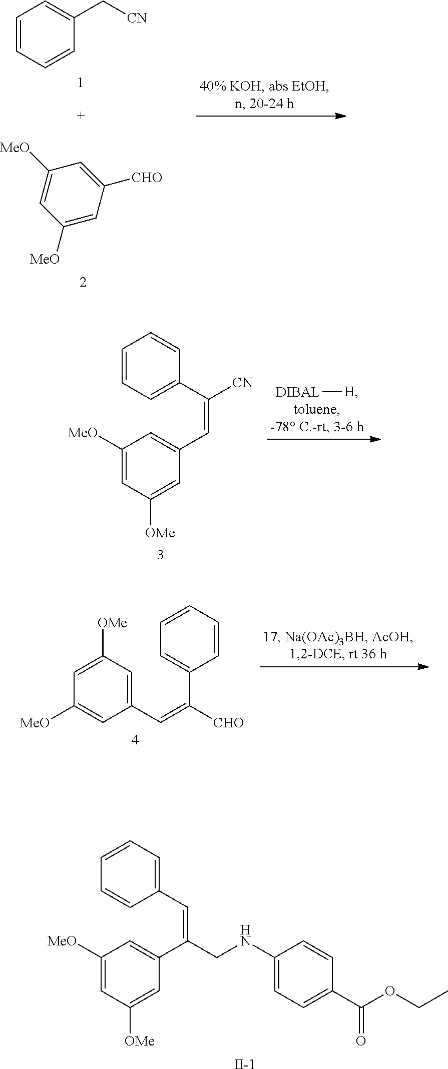

### Example 2. Synthesis Scheme of Ethyl (E)-4-((2-(3,5-Dimethoxyphenyl)-3-Phenylallyl)Amino)Benzoate (II-2)

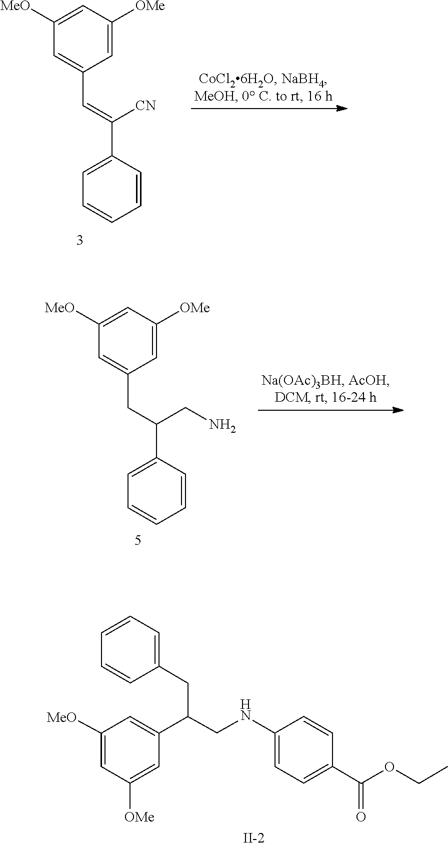 [SMILES: COc1cc(/C=C(\C#N)c2ccccc2)cc(OC)c1 ; COc1cc(CC(CN)c2ccccc2)cc(OC)c1 ; CCOC(=O)c1ccc(NCC(Cc2ccccc2)c2cc(OC)cc(OC)c2)cc1 ; "II-2"]

To synthesize ethyl 4-((2-(3,5-dimethoxyphenyl)-3-phenylpropyl)amino)benzoate (II-2), we prepared 3-(3,5-dimethoxyphenyl)-2-phenylpropan-1-amine (5) from nitrile 3 via the double reduction in addition to nitril group in the presence of CoCl₂—NaBH₄, the subsequent reductive amination of amine 5 with ethyl 4-formylbenzoate afforded the II-2 as shown in the Scheme above.

### Example 3. Synthesis of Synthesis of Ethyl (Z)-4-((2,3-Diphenylallyl)Amino)Benzoate (II-3)

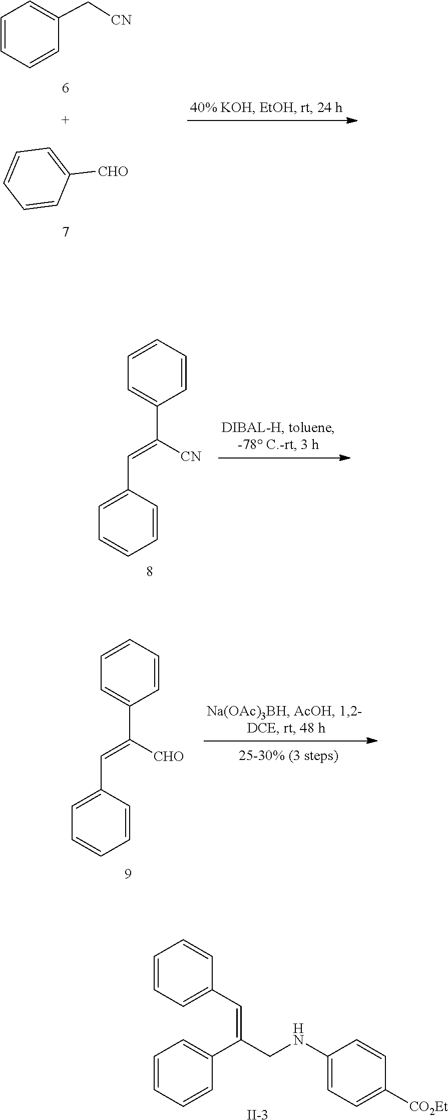

We have observed that substitution of methoxy functional group on the aromatic rings lowered the cell survival activity of II-1. Therefore, in order to increase the potency of II-1, we modified it to defunctionalize the methoxy substituent groups on the aromatic ring of the stilbene segment. In addition, we also modified the amino-benzoate functional moiety of II-1 to benzamide derivatives with the attachment of serine and threonine amino acid side chain linkers, as these pharmaceutically important pharmacophores would have a potential impact on the broadening of multi-synthetase activity at the given instance of disease target. Therefore, we synthesized the modified stilbene core without methoxy groups on both the aromatic rings by following a three-step reaction sequence, starting from simple and commercially available starting materials, benzaldehyde and 2-phenyl-acetonitrile.

The first step in the sequence involving condensation of 2-phenyl-acetonitrile (6) and benzaldehyde (7) in presence of 40% aqueous ethanolic KOH provided the 2,3-diphenylacrylonitrile (8). However, unlike routine synthesis, a significant formation of 2-phenylacetic acid from the reactant 2-phenylacetonitrile under strong basic conditions limited the reaction outcome to moderate yield. Nevertheless, the crucial reduction of CN-group to the corresponding 2,3-diphenylacrylaldehyde 9 was achieved by using a mild reducing agent, diisobutylaluminium hydride (DIBAL-H) reaction conditions. Due to the slow reactivity of CN-group at lower reaction temperature, and an unwanted double bond reduction, CN group reduction of 8 to the corresponding amine, and further reduction of formed aldehyde 9 to the corresponding alcohol at higher temperatures had greatly hindered the scale-up reaction beyond 0.1 to 0.15 gram. Therefore, we conducted a series of small-scale reactions to obtain the required quantity in order to proceed further steps.

The subsequent reductive amination of the obtained 2,3-diphenylacrylaldehyde 9 with ethyl-4-aminobenzoate under sodium triacetoxyhydroborate (NaBH(OAc)₃ conditions afforded the pure stilbene-aminobenzoate conjugate II-3 in 25-30% yield over three steps.

### Example 4. Synthesis of (E)-N1-(2,3-Diphenylallyl)Benzene-1,4-Diamine (II-5) AND (E)-4-((2,3-Diphenylallyl)Amino)-N-Hydroxybenzamide (II-6)

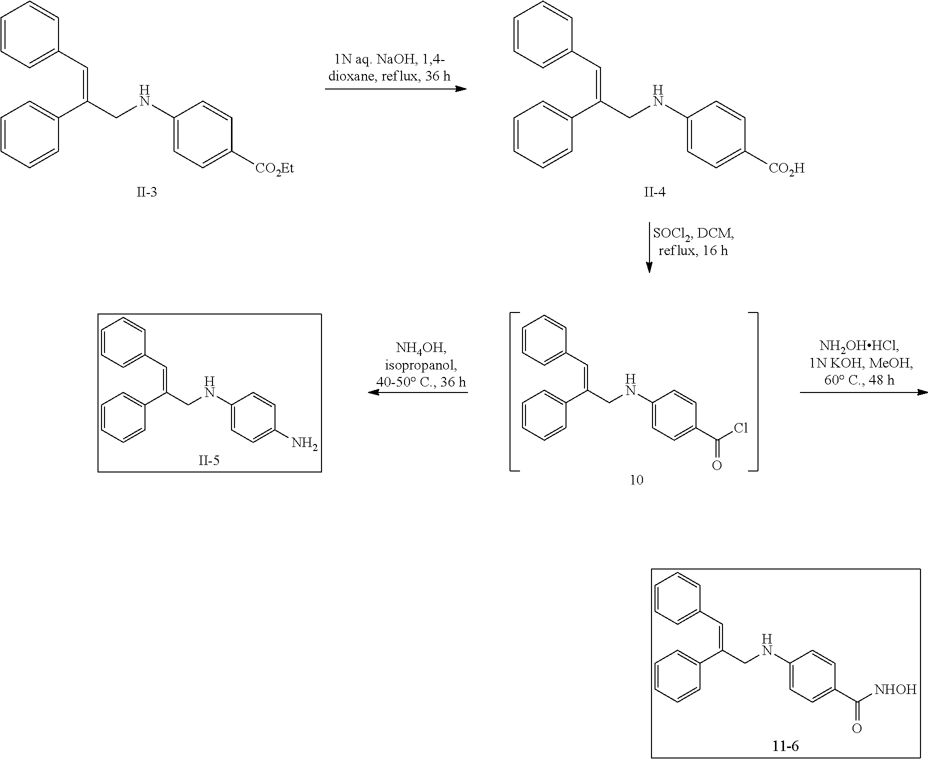

To prepare II-5, we initially treated II-3 with 28% aqueous ammonia (NH₄OH) in isopropanol, refluxing at room temperature. Unfortunately, under these reactions condition the desired product could not be obtained. However, the conversion of ester to its acid chloride 10 followed by the treatment of crude reaction mixture with NH₄OH in isopropanol resulted in the desired benzamide derivative II-5 in good yield. Similarly, the treatment of II-3 with hydroxylamine hydrochloride in presence of 1N KOH in methanol furnished the hydroxamic acid derivative II-6 in good yield as shown in the above scheme.

### Example 5. Synthesis Scheme of II-7 and II-8: Synthesis of 4-((2,3-Diphenylallyl)Amino)-N-(2-Hydroxyethyl)Benzamide (II-7) and 4-((2,3-Diphenylallyl)Amino)-N-(2-Hydroxypropyl)Benzamide (II-8)

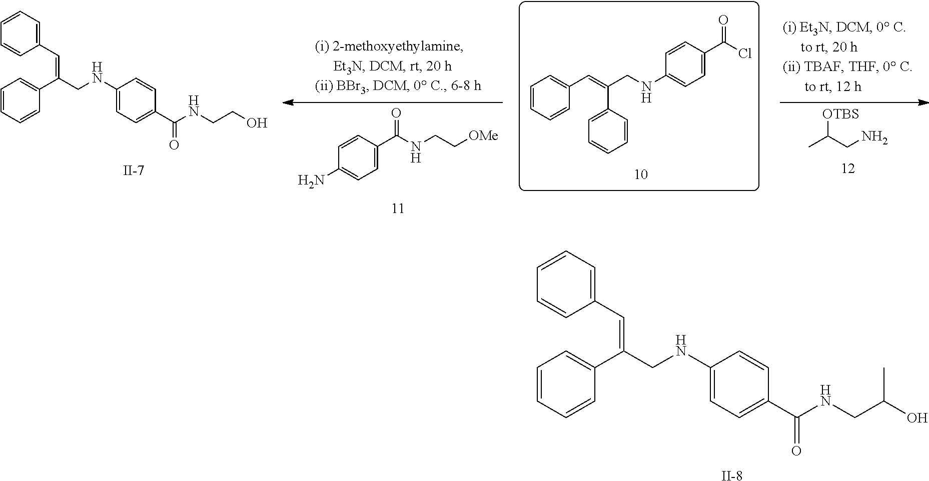

To connect a serine amino acide side chain to II-3, we initially synthesized the ethanolamine connected benzamide which upon reductive amination should provide the desired product. Nevertheless, our efforts with the treatment of 9 with the benzamide 11 was unsuccessful under reductive amination reaction conditions. Therefore, we have choosen the acid-amine coupling approach by converting the estre to acid chloride, and then treating the acid chloride with 2-methoxyethylamine under basic conditions to provide the desired II-7 in good yield over four steps.

A similar approach with 2-methoxypropan-1-amine was ineffective to provide the respective amide derivative II-8. However, the TBS-protected 1-aminopropan-2-ol (12) under acid-amine coupling reaction conditions furnished the desired threonyl side chain linker of the benzamide II-8 as depicted in the Scheme. Interestingly, the II-8 (EC₅₀=≤25 nM) is more potent than the II-3 (EC₅₀=≥25 μM) as shown in MTT assay.

¹H NMR for compound II-8 (400 MHZ, Chloroform-d) δ 7.61-7.52 (m, 2H), 7.30-7.19 (m, 3H), 7.14-7.06 (m, 2H), 7.01 (dd, J=4.9, 1.9 Hz, 3H), 6.85 (dd, J=6.8, 3.0 Hz, 2H), 6.55-6.51 (m, 3H), 4.30 (t, J=6.0 Hz, 1H), 4.05 (d, J=4.5 Hz, 2H), 3.90 (ddq, J=12.5, 6.2, 2.9 Hz, 1H), 3.50 (ddd, J=14.0, 6.4, 2.9 Hz, 1H), 3.27 (s, 1H), 3.19 (ddd, J=14.0, 7.6, 5.2 Hz, 1H), 1.12 (d, J=6.3 Hz, 3H). ¹³C NMR (101 MHz, Chloroform-d) δ 168.59, 150.72, 139.09, 138.38, 136.31, 129.18, 128.91, 128.80, 128.56, 127.98, 127.64, 127.50, 126.87, 122.42, 112.17, 67.82, 51.60, 47.58, 20.99. HRMS (ESI) calcd for C₂₅H₂₇N₂O₂, 387.2073; found, 387.2069.

### Example 6. Picomolar Aβ40 and Aβ42 Protect Against Tyrosine-Mediated TyrRS Depletion in Cortical Neurons

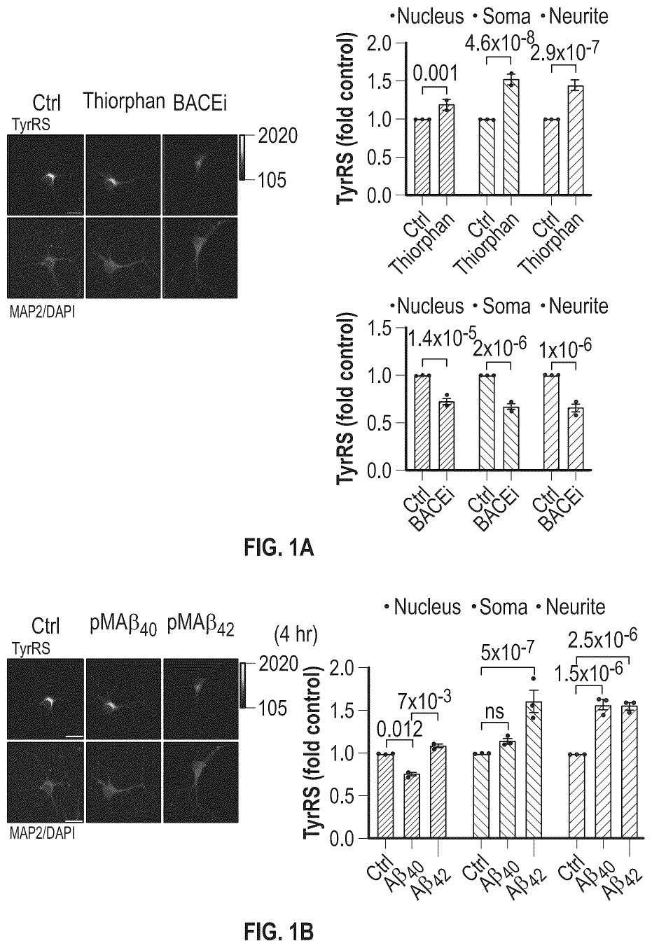

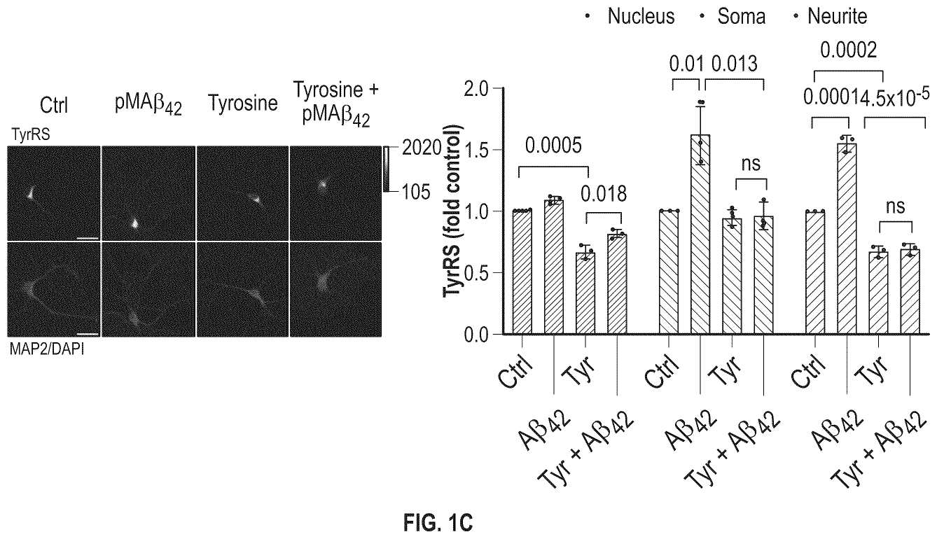

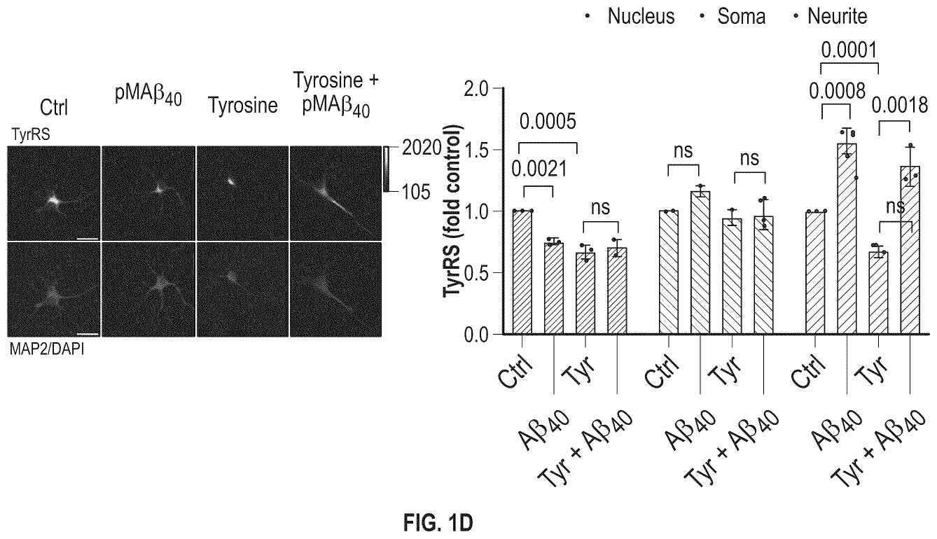

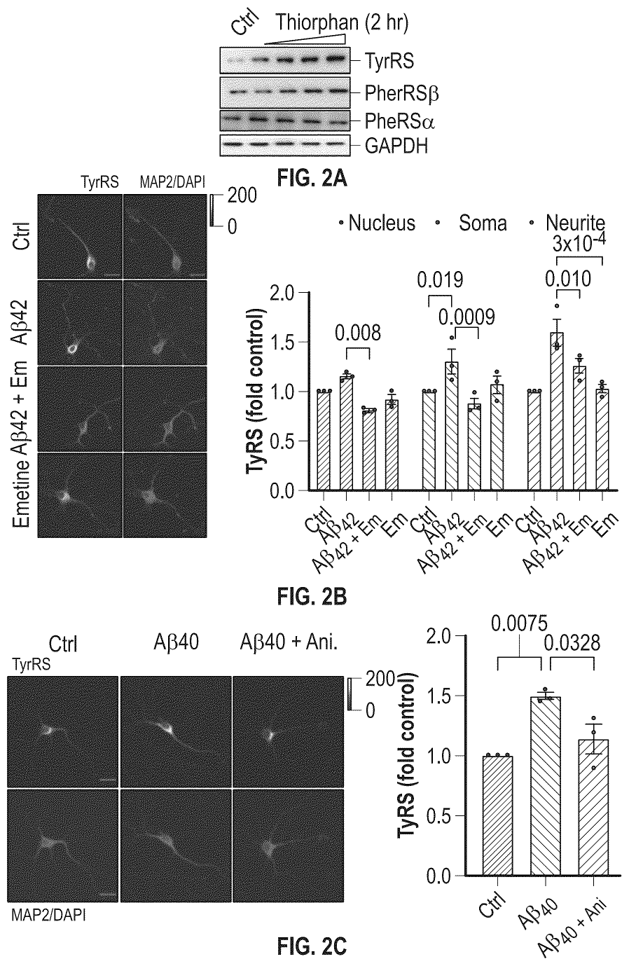

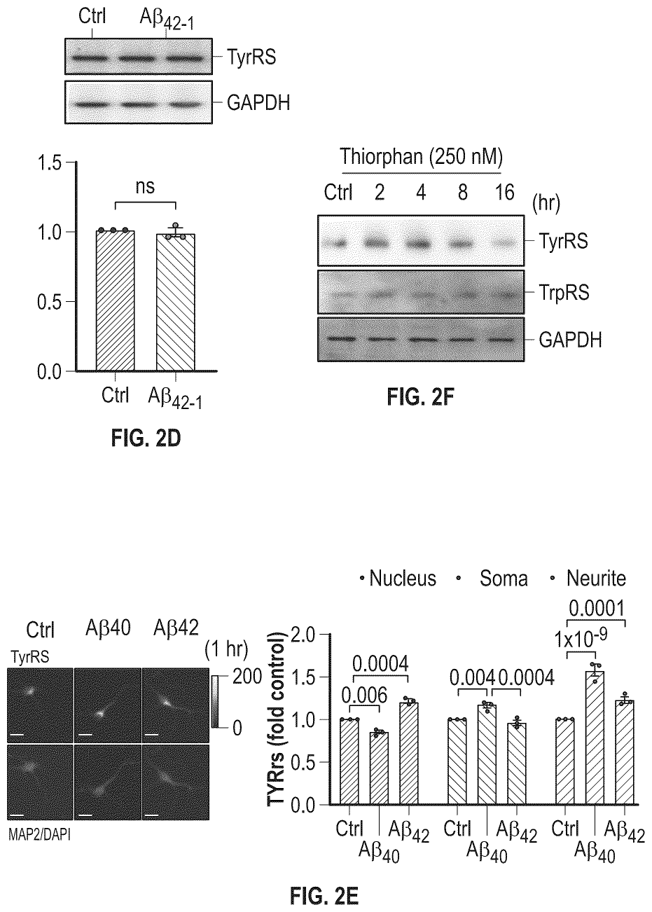

TyrRS is essential for protein synthesis, but it also has moonlighting functions especially in the regulation of PARP1-dependent nuclear functions. To determine the effect of endogenous Aβ levels on neuronal TyrRS levels, rat cortical neurons (DIV 9/10) were treated with thiorphan, an inhibitor of neprilysin that degrades Aβ peptides. It was found that thiorphan increased TyrRS levels (FIG. 1A) along with PheRSβ (FIG. 2A) but not PheRSα levels. Conversely, treatment with verubecestat, a BACE-1 inhibitor that causes dose-dependent cognitive decline in AD patients, decreased neuronal TyrRS levels (FIG. 1A).

To determine whether the effects of thiorphan and verubecestat were due to the changes in the endogenous Aβ levels, primary rat cortical neurons (DIV 9/10) were treated with picomolar amounts of Aβ (pMAβ) peptides (50 pM) and found that pMAβ increased TyrRS levels in the neurites (FIG. 1B) in a protein synthesis-dependent manner (FIGS. 2B and C). In contrast, a reverse peptide of Aβ42 (Aβ₄₂₋₁) did not show any effects (FIG. 2D). pMAβ40 and pMAβ42 exhibited opposing effects on the nuclear TyrRS levels (FIG. 1B). While treatment with pMAβ40 sustained the nuclear depletion (FIG. 1B), Aβ42 transiently increased nuclear TyrRS levels at 1 hr (FIG. 2E) and returned to control levels in 4 hr (FIG. 1B), suggesting that pMAβ40 and pMAβ42 may evoke opposing effects on the nuclear functions of neuronal TyrRS. However, prolonged treatment with thiorphan depleted TyrRS in 16 hr (FIG. 2F), whereas there was an increase in tryptophanyl-tRNA synthetase (TrpRS) which is an activator of PARP1 during inflammatory response (FIG. 2F). Together, these data indicate that endogenous Aβ may have dual effects on neuronal TyrRS levels in a concentration-dependent manner. At picomolar concentrations, Aβ increases TyrRS levels, but it may facilitate neuronal TyrRS depletion at higher concentrations.

Previously it was observed that tyrosine depletes neurite and nuclear TyrRS levels and inhibits protein synthesis at the elongation step. Therefore, whether pMAβ would protect against tyrosine-mediated TyrRS depletion was investigated. To do so, rat cortical neurons (DIV 9/10) were treated with tyrosine, followed by pMAβ peptides for 4 hr. Although pMAβ42 did not prevent tyrosine-induced TyrRS depletion in the neurites, it rescued nuclear TyrRS levels (FIG. 1C). In comparison, pMAβ40 rescued neurite TyrRS levels without affecting its nuclear or soma levels (FIG. 1D). Collectively, these findings indicate that pMAβ40 and pMAβ42 modulate nuclear and neurite TyrRS levels through distinct mechanisms, and tyrosine disrupts the functions of both Aβ peptides.

### Example 7. Picomolar Aβ Stimulates Neuronal Protein Synthesis in Cortical Neurons

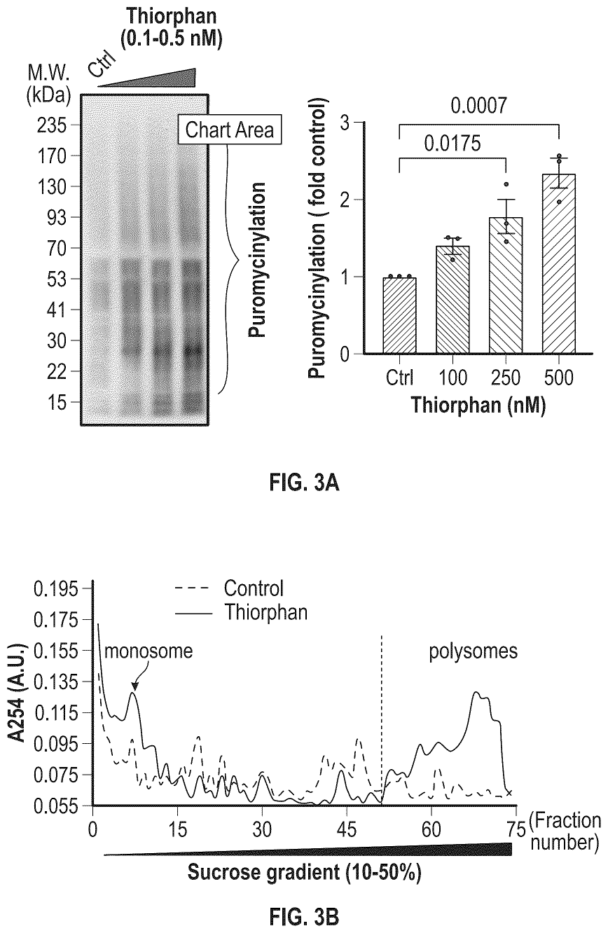

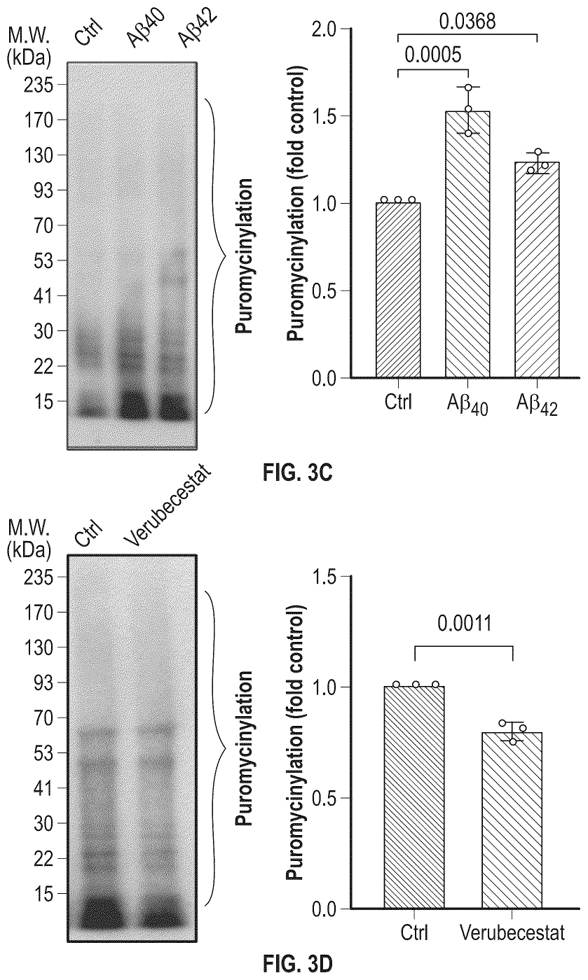

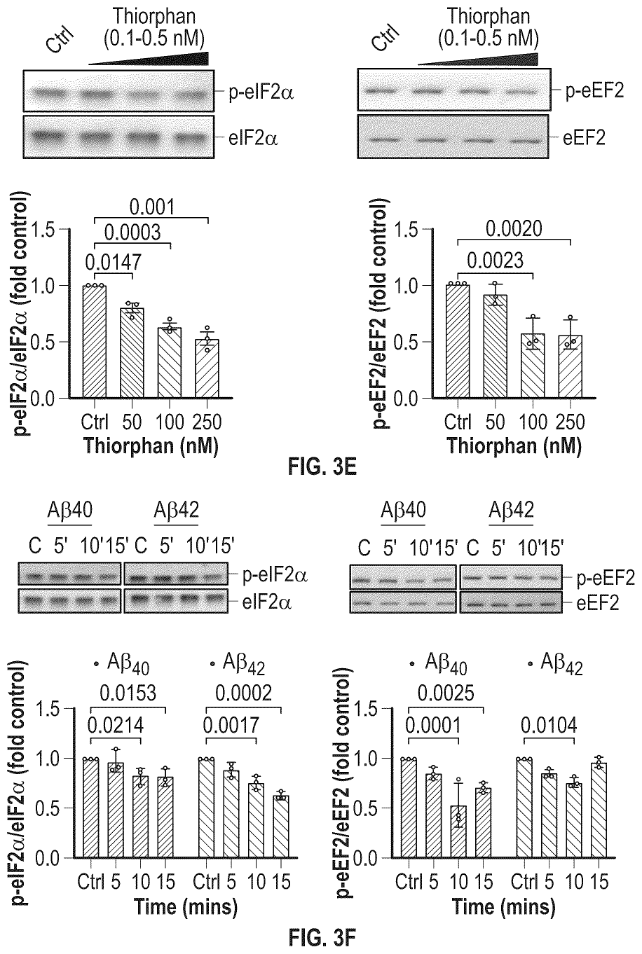

Neuronal protein synthesis is regulated in an activity-dependent manner and protein synthesis is a ubiquitous feature of synaptic compartments. Phosphorylation of eukaryotic initiation factor 2 alpha (p-eIF2a) and phosphorylation of eEF2 by eEF2K inhibit protein synthesis at the initiation and elongation steps, respectively. Although activation of eIF2α stimulates memory formation, intriguingly, regulated phosphorylation of eIF2α is also required for normal cognitive and motor functions in non-pathological state. BDNF and dopamine (DA)-dependent activation of the mammalian target of rapamycin (mTOR) inhibits eEF2K to activate protein synthesis. Since thiorphan and verubecestat have opposite effects on TyrRS levels, without wishing to be bound by theory, it was hypothesized that treatment with thiorphan and Aβ would stimulate neuronal protein synthesis while verubecestat might inhibit it. Consistently, treatment with thiorphan activated neuronal protein synthesis as measured by the puromycin incorporation assay (FIG. 3A) and polysome profiling (FIG. 3B). Further, treatment with both pMAβ40 and pMAβ42 also stimulated protein synthesis as determined by the puromycin incorporation assay (FIG. 3C) while treatment with verubecestat decreased puromycin incorporation in rat cortical neurons (FIG. 3D). Further, it was determined whether thiorphan and Aβ would modulate p-eIF2α and p-eEF2 levels. Consistent with its ability to stimulate global protein synthesis, thiorphan decreased p-eIF2α and p-eEF2 levels (FIG. 3E). Similarly, pMAβ40 and pMAβ42 also decreased p-eIF2α and p-eEF2 levels (FIG. 3F). Since p-eIF2α and p-eEF2 levels are increased in AD brains, together these results indicate that Aβ stimulated neuronal protein synthesis is attenuated in AD brains.

### Example 8. Tyrosine and Phenylalanine Distinctly Regulated TOP1 and TOP2β Induced DNA Breaks and Associated Gene Expression in Neurons

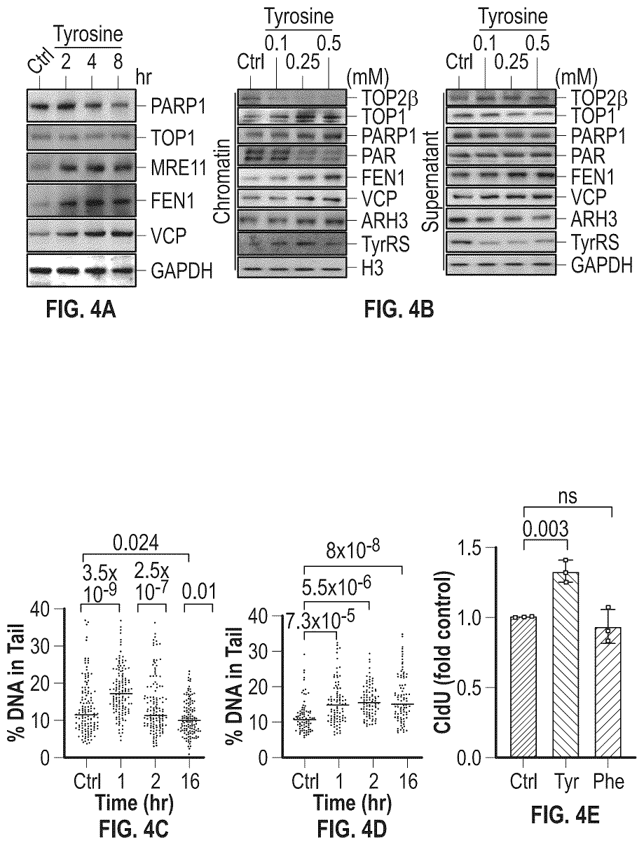

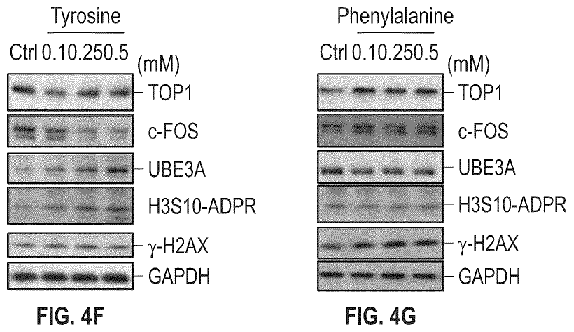

Although tyrosine is known to induce DNA damage, the mechanism of tyrosine-mediated DNA damage induction is not known. Moreover, L-tyrosine is not an inducer of neuorodegeneration despite its ability to induce neuronal DNA damage, suggesting that treatment with L-tyrosine may induce the activation of DNA repair proteins. Since topoisomerases are major mediators of DNA damage in neurons, we hypothesized that tyrosine and phenylalanine would distinctly regulate topoisomerases-mediated DNA damage in neurons as well. To test this hypothesis, cortical neurons treated with tyrosine (500 μM) for up to 8 hr and the changes in PARP1, TOP1, MRE11, FEN1, and VCP levels were determined by WB using their corresponding antibodies (FIG. 4A). This data showed that increased levels of tyrosine can induce the expression of DNA repair proteins. To test if tyrosine would recruit the DNA factors to the chromatin to facilitate DNA repair, we performed chromatin separation assay after treatment with tyrosine for 15 min. We treated cortical neurons treated with increasing concentrations of tyrosine (0.1-0.5 mM) for 15 min and association of TOP1, TOP2β, PARP1, PAR, FEN1, VCP, ARH3, TyrRS with the chromatin were determined using their specific antibodies (FIG. 4B). This data suggested that tyrosine treatment can indeed stimulate DNA repair in neurons. To confirm this observation, we performed alkaline comet assay (FIG. 4C). Primary cortical neurons (DIV 9/10) treated with tyrosine (500 μM) for up to 16 hr and the percentages of DNA in the comet tail were quantified. Interestingly, treatment with phenylalanine (500 μM) for up to 16 hr sustained the DNA damage (FIG. 4D). Furthermore, nucleotide pulse labeling using 50 μM of nucleoside analog, CldU (5-chloro-2′-deoxyuridine) also showed that tyrosine but not phenylalanine induces DNA repair in neurons (FIG. 4E). To determine if tyrosine-mediated recruitment of TOP1 and removal of TOP2β from the chromatin (FIG. 4B) results in corresponding gene expression changes, we determined the changes in the levels of UBE3A and H3-Ser-ADPR (TOP1 target and PARP1 trapping) and c-FOS and γ-H2AX (TOP2β target and DSB marker). Primary cortical neurons (DIV 9/10) treated with tyrosine (100-500 μM) for 8 hr and changes in the levels of TOP1, c-FOS, UBE3A, H3-Ser-ADPR, and γ-H2AX levels were determined by WB using their specific antibodies (FIG. 4F). Similarly, primary cortical neurons (DIV 9/10) treated with phenylalanine (100-500 μM) for 8 hr and changes in the levels of TOP1, c-FOS, UBE3A, H3-Ser-ADPR, and γ-H2AX levels were determined by WB using their specific antibodies (FIG. 4G). Together these data indicate that tyrosine and phenylalanine distinctly regulate TOP1 and TOP2β-induced DNA breaks.

### Example 9. Picomolar Aβ Stimulates Neuronal Transcription and Rescues Tyrosine-Mediated Transcriptional Inhibition in Cortical Neurons

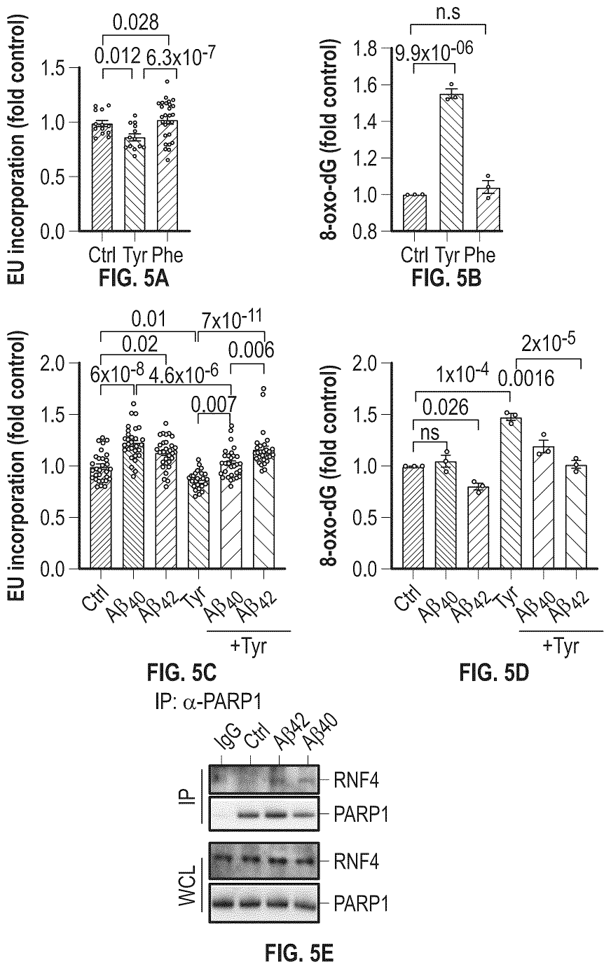

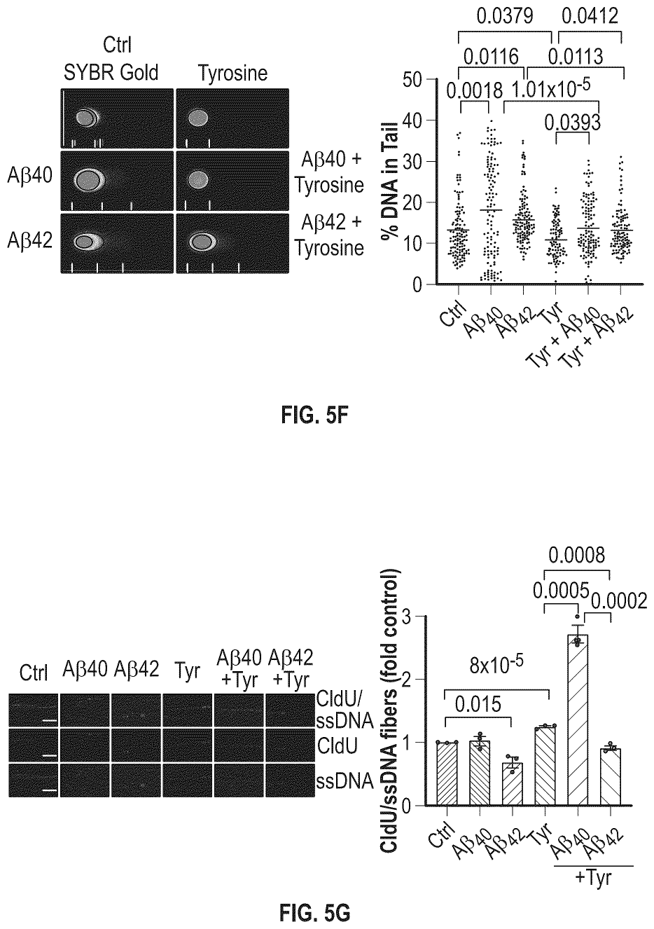

Oxidative DNA damage ‘traps’ PARP1 and inhibits transcription and PARP1 removal from the chromatin by the concerted actions of valosin-containing protein (VCP) and RNF4-depent ubiquitination are required to facilitate transcription. Previously it was observed that tyrosine induces oxidative DNA damage, potentially by depleting TyrRS. Therefore, we hypothesized that tyrosine will inhibit global transcription. Therefore, rat cortical neurons were treated with tyrosine for 16 hr and measure the levels of transcription using EU incorporation and found that tyrosine inhibits global transcription (FIG. 5A). However, phenylalanine in contrast increased transcription, suggesting that tyrosine and phenylalanine have differential effects of global transcription activation. Since tyrosine and phenylalanine have differential effects on neuronal oxidative DNA damage (FIG. 5B), without wishing to be bound by theory, it was hypothesized that tyrosine and phenylalanine would have differential effects on neuronal transcription and both pMAβ40 and pMAβ42 would rescue tyrosine-mediated transcription inhibition by activating the repair of oxidative DNA damage. To test this hypothesis, the effect of tyrosine alone or in combination with pMAβ40 and pMAβ42 on transcription was analyzed using a 5-ethynyluridine (EU) incorporation assay. To do so, primary cortical neurons were treated with pMAβ40 and pMAβ42 for up to 3 hr and then treated with EU for 30 min in neuronal cultures pre-treated with tyrosine that induces oxidative stress. The extent of EU incorporation in nascent RNA was quantified and found while Aβ-treated neurons show increased incorporation of EU, tyrosine decreased the incorporation of EU whereas phenylalanine increased EU incorporation suggesting that tyrosine and phenylalanine have opposing effects on global transcription (FIG. 5A). Although co-treatment with pMAβ40 and pMAβ42 rescued tyrosine-mediated transcription inhibition (FIG. 5C), it was observed that pMAβ40 only partially rescued tyrosine-mediated transcription inhibition (FIG. 5C) whereas Aβ42 completely rescued it (FIG. 5C). Intriguingly, studies herein revealed that unlike tyrosine, phenylalanine did not evoke oxidative DNA damage (FIG. 5B), suggesting that tyrosine and phenylalanine have differential effects on neuronal oxidative DNA damage accumulation. To determine if pMAβ40 and pMAβ42 would rescue tyrosine-mediated oxidative DNA damage induction, the levels of 8-oxo-gyanosine (8-oxo-dG) levels were quantified in presence of tyrosine alone or in combination with pMAβ40 and pMAβ42. Interestingly, tyrosine increased the 8-oxo-dG levels and treatment with pMAβ40 and pMAβ42 rescued tyrosine-induced oxidative DNA damage (FIG. 5D). Together these observations provide a potential molecular basis for picomolar Aβ-mediated rescue of tyrosine-mediated transcription inhibition. Since trapped PARP1 inhibits transcription and both Aβ peptides induced transcription (FIG. 5D), it was determined whether Aβ peptides would facilitate the interaction of PARP1 with RNF4 and VCP. To test this possibility, PARP1 was immunoprecipitated after treatment with Aβ40 and Aβ42 and found that both Aβ40 and Aβ42 facilitated the interaction of PARP1 with RNF4 (FIG. 5E), providing a potential molecular basis for pMAβ-mediated activation of transcriptional activation by RNF4 mediated removal of PARP1 from the chromatin while facilitating the repair of oxidative DNA damage. Since both pMAβ40 and pMAβ42 rescued tyrosine-mediated transcription inhibition (FIG. 5C) and oxidative DNA damage (FIG. 5D), it was hypothesized that pMAβ40 and pMAβ42 would show differential effect on tyrosine-mediated DNA repair. To test this hypothesis, the cortical neurons were pre-treated with tyrosine for 16 hr followed by treatment with Aβ40 and Aβ42 for 1 hr and subjected the neurons to alkaline comet assay. Consistent with their ability to induce DNA damage, both Aβ40 and Aβ42 induced neuronal DNA damage even in the presence of tyrosine (FIG. 5F). Since tyrosine showed increased synthesis associated repair (SAR) in the DNA fiber assay (FIG. 4E), we further determined the effect of pMAβ40 and pMAβ42 on tyrosine stimulated SAR using a DNA fiber assay. Notably, it was found that pMAβ40 and pMAβ42 exhibited opposing effects on SAR; Aβ40 by itself showed no effect on SAR while exacerbating tyrosine-mediated SAR, whereas Aβ42 inhibited SAR even in the presence of tyrosine (FIG. 5G), indicated that pMAβ42 has a unique ability to induce neuronal DNA damage even under elevated tyrosine levels (FIGS. 5F and G).

### Example 10. pMAβ40 and pMAβ42 Distinctly Regulate Topoisomerase 1 and 2β Levels on the Chromatin in Cortical Neurons

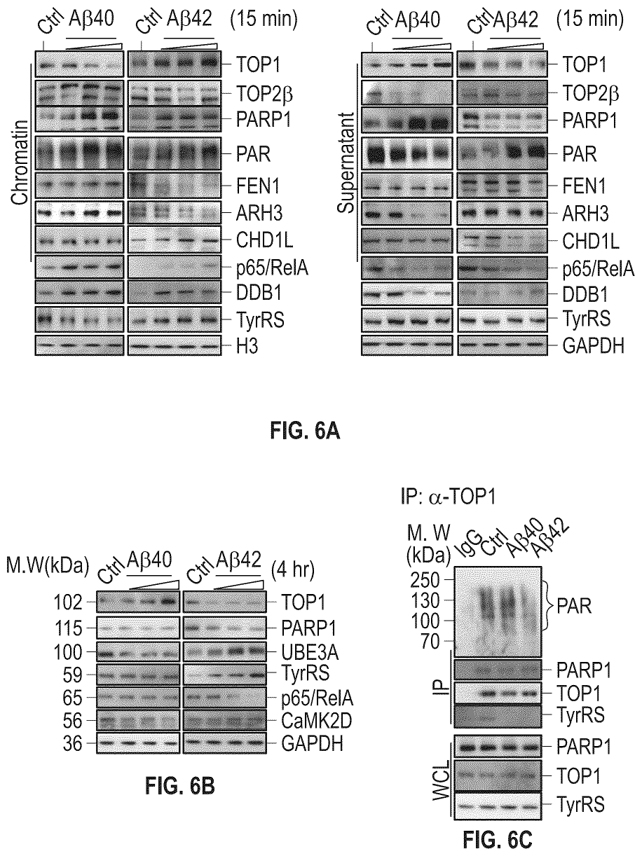

Although TOP1 is an inhibitor of transcription overactivation, it is essential for transcription associated with normal synaptic transmissions and TOP2β stimulates activity-dependent gene expression. However, unlike etoposide (ETO) that activates transcription, treatment with camptothecin (CPT) impairs transcription associated with synaptic function, suggesting that TOP1 induced SSBs and TOP2β-induced DSBs distinctly regulate neuronal transcription. Since BACE-1 inhibitors caused cognitive impairments in AD patients and TOP2β and TOP1-induced DNA breaks are essential for cognitive function and memory formation, without wishing to be bound by theory, it was hypothesized that Aβ might recruit TOP1 and TOP2β to the chromatin to activate neuronal transcription associated with cognition and memory. To determine this possibility, rat cortical neurons (DIV 9/10) were treated with pMAβ40 and pMAβ42 for 15 min and subjected them to chromatin separation. It was found that Aβ42 facilitated the recruitment of TOP1 along with TyrRS, and PARP1 without affecting TOP2β levels. Also, it was determined the chromatin's poly-ADP-ribos(PAR)ylation levels as an indicator of DNA damage-dependent PARP1 activation. In contrast, Aβ40 recruited TOP2β and PARP1 with concomitant depletion of chromatin-associated TyrRS and TOP1 (FIG. 6A). Together, these results indicate that picomolar Aβ40 and Aβ42 distinctly regulate TOP1 and TOP2β induced DNA damage response to regulate neuronal transcriptional activities. Since pMAβ42 recruit TOP1, which regulates transcription associated with normal synaptic transmissions and cognitive functions, together these findings provide a potential molecular basis for pMAβ42-mediated memory formation.

### Example 11. pMAβ42 Decreases PARP1, NF-κB, and TOP1 Protein Levels in Cortical Neurons

Since pMAβ40 and pMAβ42 stimulated transcription (FIG. 5C) and facilitated the recruitment of TOP2β and TOP1 respectively and the development of Aβ plaques decrease Aβ42/40 ratio, it was determined whether pMAβ40 and pMAβ42 would differentially regulate NF-kB-dependent transcription and PARP1 and TOP1 protein levels. To test this possibility, cortical neurons were treated with pMAβ40 and pMAβ42 and determined the protein levels of PARP1, p65/NF-kB, and TOP1. While pMAβ40 increased TOP1 protein levels, pMAβ42decreased the levels of PARP1, p65/NF-KB, and TOP1 (FIG. 6B). Moreover, pMAβ42 also increased UBE3A and CaMK2D levels whereas pMAβ40 decreased their levels (FIG. 6B).

### Example 12. Aβ40 and Aβ42 Exhibit Opposing Effects on TOP1 Induced DNA Breaks in Cortical Neurons

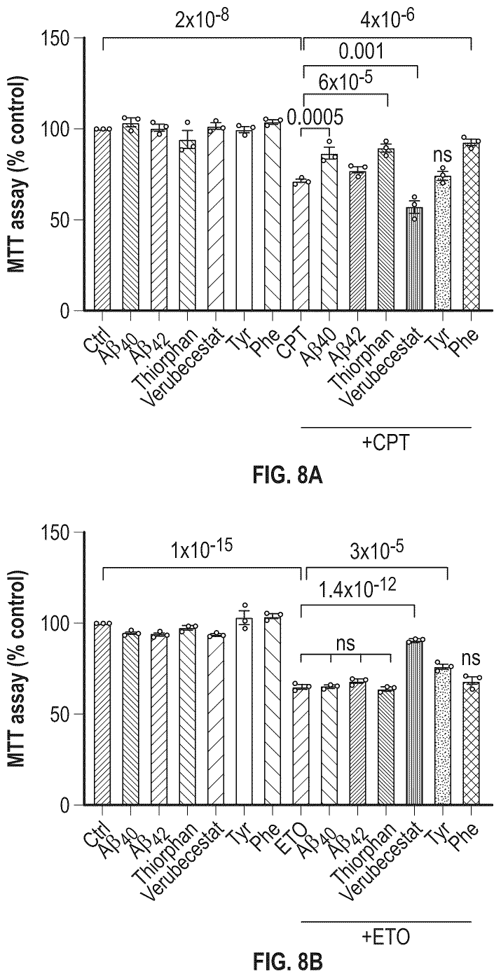

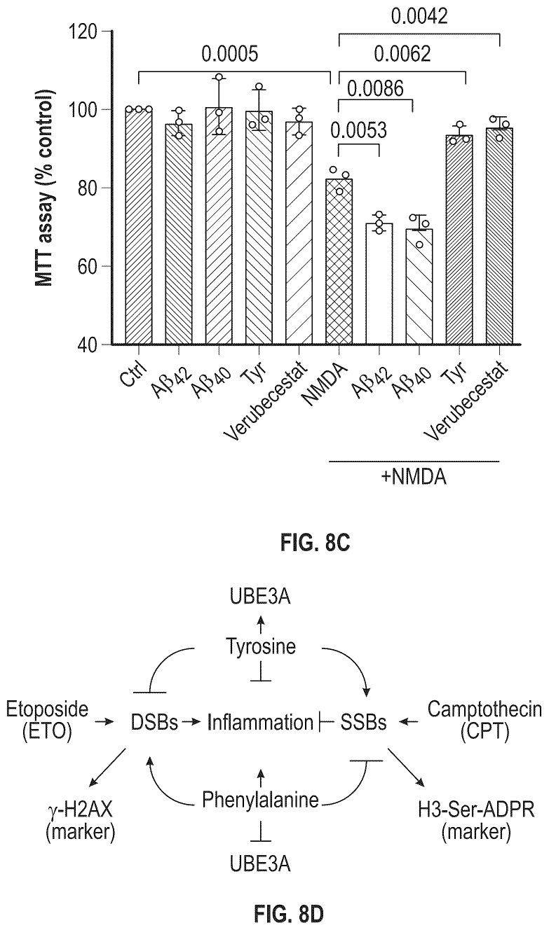

Although Aβ40 and Aβ42 exhibited differential effect on the stability of TOP1 (FIG. 6B), whether this differential effect would have any physiological effect on TOP1-mediated DNA damage response was explored. Since PARP1-dependent PARylation protects TOP1 from degradation, without wishing to be bound by theory, it was hypothesized that Aβ42 would stimulate TOP1 degradation through dePARylation. To test this possibility, TOP1 was immunoprecipitated. While Aβ42 facilitated the dePARylation of TOP1, in contrast, Aβ40 increased its PARylation levels (FIG. 6C), potentially explaining the opposing effects of Aβ40 and Aβ42 on TOP1 protein levels (FIG. 6C). PARylation of TOP1 promotes DNA repair, and inhibition/depletion of PARP1 exacerbates CPT-induced toxicity. Based on these observations, without wishing to be bound by theory, it was hypothesized that Aβ40 mediated PARylation and stabilization of TOP1 (FIG. 6C) would protect against CPT-induced neurotoxicity and DNA damage. In contrast, Aβ42-induced depletion of PARP1 (FIG. 6C) and dePARylation of TOP1 (FIG. 6C) may hinder the repair of CPT-induced DNA damage. Although PARP1 inhibition or knockdown of PARP1 exacerbates CPT induced cytotoxicity, the effect of TyrRS knockdown on CPT-induced neurotoxicity is not yet known. To corroborate the differential effect of Aβ40 and Aβ42 on CPT-induced neurotoxicity (β), we quantified the extent of CPT-induced DNA breaks using an alkaline comet assay. To test this hypothesis, the effect of Aβ40 and Aβ42 on CPT-induced neurotoxicity was determined. Therefore, we tested the effect of siRNA knockdown of both TyrRS and PARP1 on CPT-induced toxicity and Aβ40-mediated neuroprotection. Consistent with previous reports that PARP1 protects against CPT, and that TyrRS is essential for neuronal survival, it was found that siRNA knockdown of either TyrRS or PARP1 exacerbated CPT-induced neurotoxicity, and Aβ40 protected against CPT in a TyrRS and PARP1-dependent manner (β) (FIG. 6D). However, supporting Aβ40-mediated PARylation of TOP1 (FIG. 6C), neuronal cultures treated with CPT in combination with Aβ40 showed reduced DNA in the comet tail whereas Aβ42 had no effect (FIG. 6E). Furthermore, cortical neurons were treated with CPT alone or in combination with Aβ40 or Aβ42, thiorphan or verubecestat, tyrosine or phenylalanine and quantified the neuronal survival using the MTT assay. Aβ40, thiorphan, and phenylalanine protected against CPT-induced neurotoxicity, while verubecestat exacerbated it (FIG. 8A). However, tyrosine and Aβ42 did not show any effect on CPT-induced neurotoxicity (FIG. 8A), underscoring the essential role of Aβ40 and phenylalanine in protecting against TOP1 induced neuronal DNA damage. Together these results suggest that Aβ40 and Aβ42 exhibit differential effects in the repair of CPT-induced DNA damage, nuclear TyrRS, and TOP1 levels.

### Example 13. Picomolar Aβ42 Protects Against Tyrosine-Phenylalanine Combination Induced Neurite Degeneration

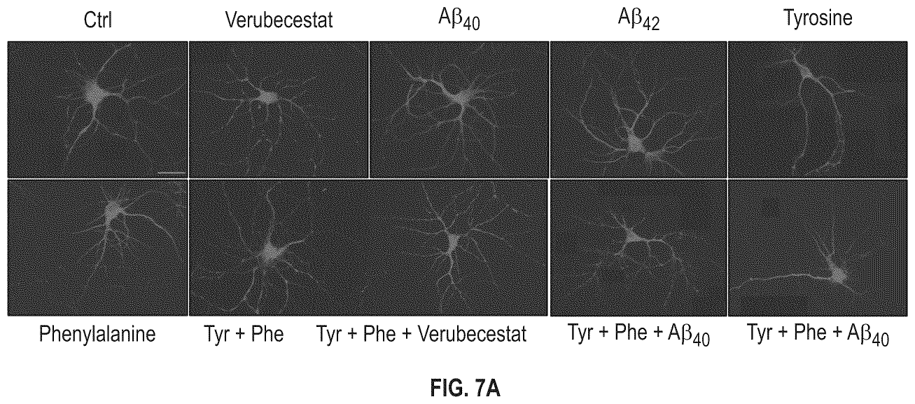

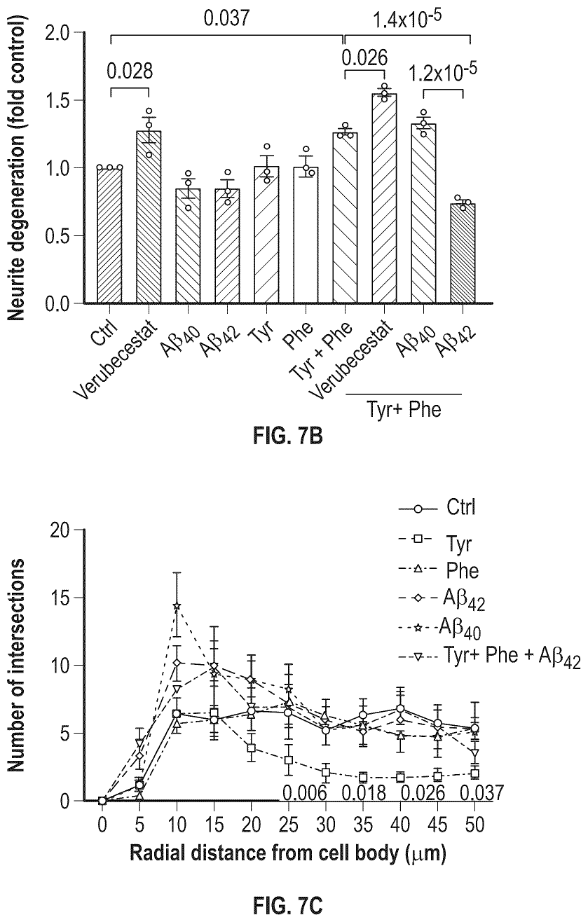

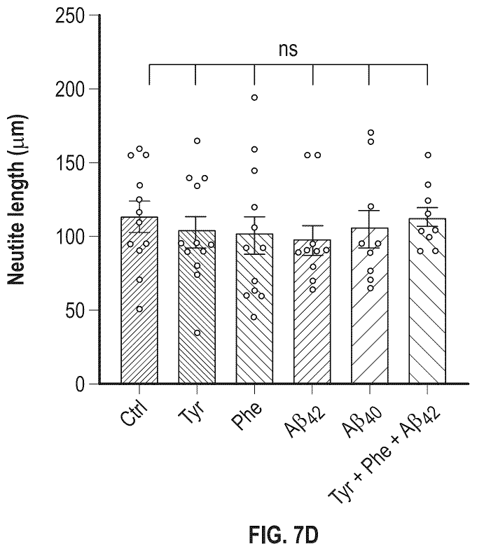

Since phenylalanine/Aβ40 protected against CPT (FIG. 6D), we hypothesized a combination of both tyrosine and phenylalanine or prolonged treatment with verubecestat might inhibit topoisomerase-dependent transcription to trigger neurite degeneration and only Aβ42 that stimulates neuronal DNA damage in presence of tyrosine (FIGS. 4F and 4G) would protect against it. To test this hypothesis, we treated cortical neurons with either tyrosine or phenylalanine alone or in combination (Tyr-Phe) with or without verubecestat 24 hr and then treated them with Aβ40 or Aβ42 (50 pM) for another 4 hr in cultures and quantified the levels of neurite degeneration. We found that while sustained presence of elevated tyrosine or phenylalanine alone did not evoke significant neurite degeneration, Tyr-Phe combination induced neurite degeneration, and in combination with verubecestat exacerbated it (FIG. 7A). Interestingly, we found that only picomolar Aβ42 but not Aβ40 protected against Tyr-Phe combination-induced neurite degeneration (FIGS. 7B and 7C), suggesting an essential function of picomolar Aβ42 in protecting against Tyr-Phe combination induced neurite degeneration. However, neither Aβ40 nor Aβ42 (50 pM) themselves had any effect on neurite stability for up to 24 hr (FIG. 7D).

### Example 14. Mimicking BACE-1 Inhibitors, Tyrosine Protects Against NMDA and Etoposide-Induced Neurotoxicity

Interestingly, Aβ peptides are known to exacerbate NMDA toxicity and consistently, BACE-1 inhibitors protect against it. PARP1 is considered a major driver of neurodegeneration in multiple conditions, including excitotoxicity. Since Aβ peptides recruit topoisomerases and PARP1 to the chromatin (FIG. 6A), and encouraged by our observation that tyrosine inhibits PARP1, we hypothesized that tyrosine and Aβ peptides would exhibit opposing effects on NMDA and ETO-induced neurotoxicity and tyrosine would protect against them. Consistently, we found that treatment with tyrosine protected against NMDA and ETO-induced neurotoxicity (FIGS. 8B and 8C) whereas Aβ peptides exacerbated NMDA toxicity and showed no effect on ETO-induced neurotoxicity. Similarly, phenylalanine also did not show any effect on NMDA and ETO-induced neurotoxicity. However, in contrast to the observed exacerbation of CPT-induced neurotoxicity (FIG. 8A), verubecestat protected against both NMDA and ETO-induced neurotoxicity (FIGS. 8B and 8C), suggesting a critical role of Aβ peptides in sustaining activity-induced DSBs while protecting against neuronal SSBs-induced neurotoxicity. Taken together, tyrosine and phenylalanine may distinctly regulate single stand and double strand DNA breaks and signaling though H3-Ser-ADPR and γ-H2AX mediating inflammatory gene expression (FIG. 8D)

### Example 15. Nanomolar Aβ42 Accumulates PARP1 and Induces Neurotoxicity in Cortical Neurons

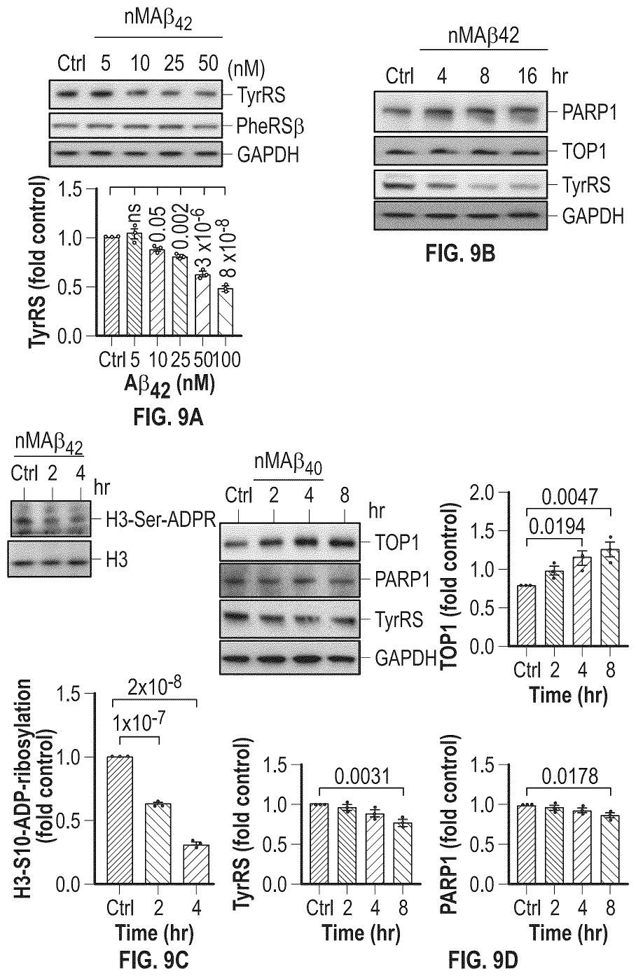

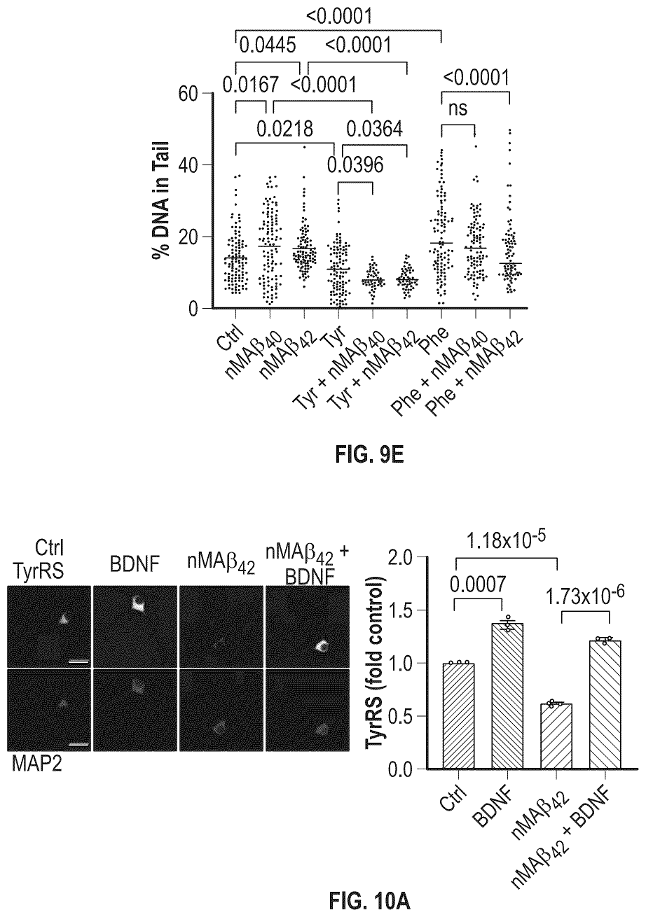

Since AD brains accumulate PARP1 and TOP1 along with TyrRS depletion, without wishing to be bound by theory, it was hypothesized that a higher concentration of Aβ40 and Aβ42 would accumulate PARP1 and TOP1 levels with concomitant TyrRS depletion. To test this hypothesis, cortical neurons were treated with 1000-fold higher levels of Aβ40 or Aβ42 (i.e; 50 nM vs 50 pM) and found that nanomolar Aβ42 (nM Aβ42) deplete neuronal TyrRS (FIG. 9A) and accumulate PARP1 along with depletion of neuronal TyrRS but not TOP1 in a time-dependent manner (FIG. 9B) and depleted H3-Ser-ADPR levels (FIG. 9C). However, nanomolar Aβ40 (nM Aβ40) accumulated TOP1 with concomitant depletion of both PARP1 and TyrRS levels (FIG. 9D). Most significantly, nM Aβ40 and 42 failed to induce DNA breaks in the presence of tyrosine (FIG. 9E).

### Example 16. Activators of Protein Synthesis Rescue nM Aβ42-Mediated TyrRS Depletion and Accumulation of DNA Damage and Neurite Degeneration

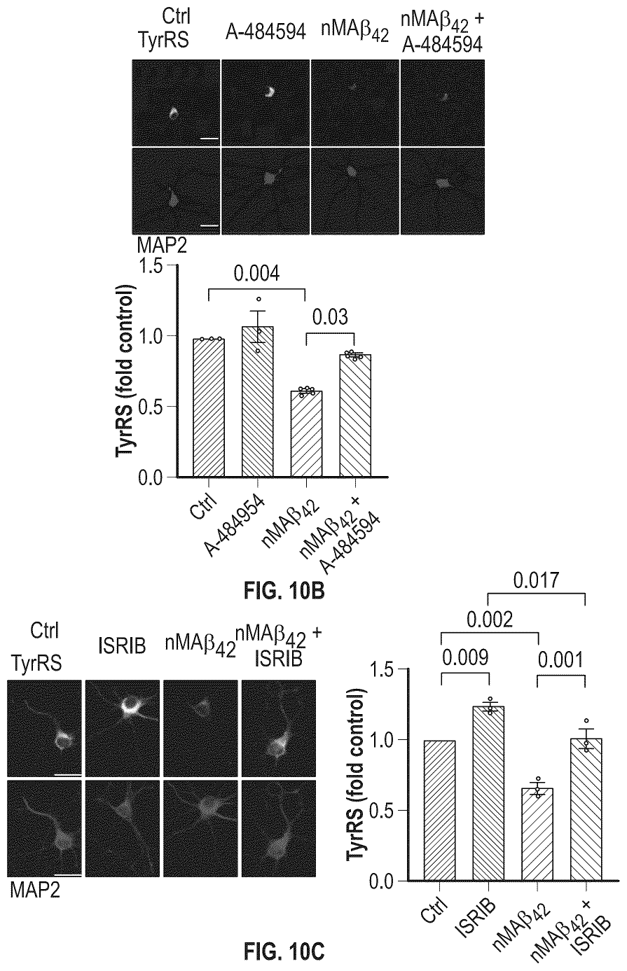

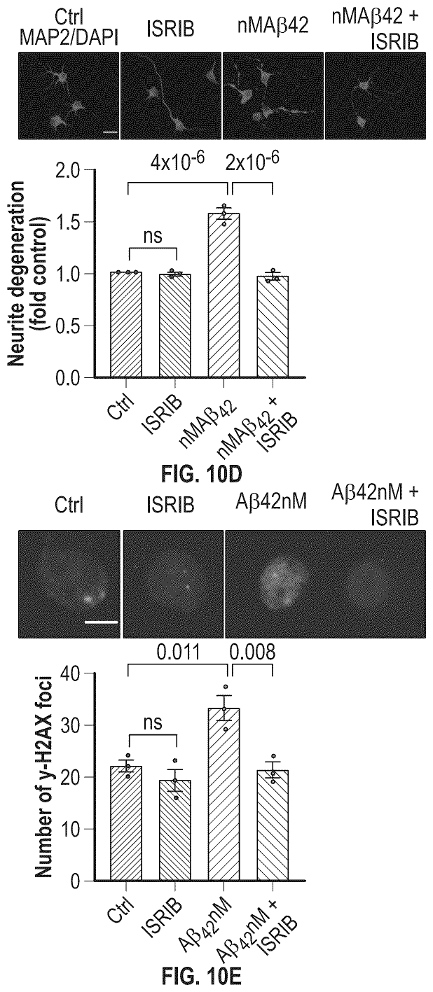

Neuronal protein synthesis is regulated in an activity-dependent manner and protein synthesis is a ubiquitous feature of neuronal synaptic compartments. Consistently, protein synthesis is increased in the brain during memory formation, suggesting that axonal TyrRS mRNA would be translated during memory formation and may thus contribute to its positive correlation with cognitive performance score. Phosphorylation of eukaryotic initiation factor 2 alpha (p-eIF2a) and phosphorylation of eukaryotic elongation factor 2 (p-eEF2) by eEF2 kinase (eEF2K) inhibit protein synthesis at the initiation and elongation steps, respectively. Therefore, dephosphorylation of p-eIF2a and p-cEF2 by protein phosphatase 1 and 2A (PP1 and PP2A), respectively activate protein synthesis. Similarly, BDNF and dopamine (DA)-dependent activation of the mammalian target of rapamycin (mTOR) inhibits eEF2K to activate protein synthesis. Although activation of elF2a regulates circadian behavior and memory formation, intriguingly, regulated phosphorylation of eIF2a is also required for normal cognitive and motor function in a non-pathological state. Consistent with the downregulation of both DA and BDNF in AD brains, p-eIF2a and p-eEF2 levels are increased while protein synthesis and TyrRS levels are decreased in AD brains. Therefore, we hypothesized that treatment with protein synthesis activators will protect against nM Aβ42-mediated TyrRS depletion, neurite degeneration and DNA damage accumulation. Therefore, we treated rat cortical neurons (DIV 9/10) with nM Aβ42 (50 nM) for 16 hr or in combination with BDNF for 1 hr and TyrRS levels were determined using anti-TyrRS antibody after treatment (FIG. 10A). Similarly, rat cortical neurons (DIV 9/10) were treated with nM Aβ42 (50 nM) for 16 hr or in combination with A-484594 (eEF2K inhibitor, 100 nM) (FIG. 10B). Most importantly, treatment with ISRIB also protected against TyrRS depletion. Rat cortical neurons (DIV 9/10) were treated with nM Aβ42 (50 nM) for 16 hr or in combination with ISRIB (100 nM) (FIG. 10C). Together these data show that reactivation of protein synthesis rescues nM Aβ42-mediated TyrRS depletion. Furthermore, treatment with ISRIB also rescued nM Aβ42-mediated neurite degeneration (FIG. 10D) and DNA damage accumulation (FIG. 10E).

### Example 17. Cis-RSV Rescues Tyrosine-Mediated Transcription Inhibition by Activating Neuronal DNA Damage Induction that Mitigates Tyrosine-Mediated DNA Repair

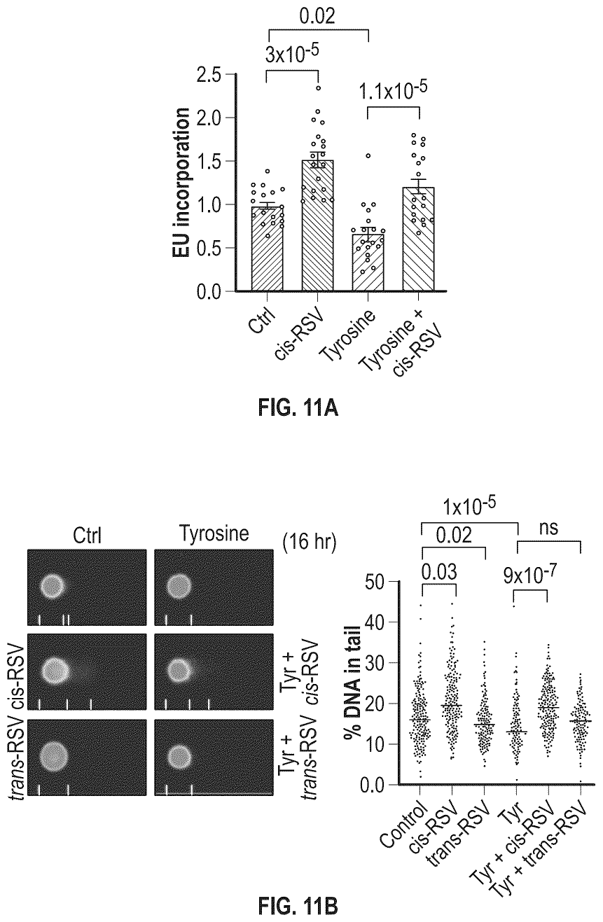

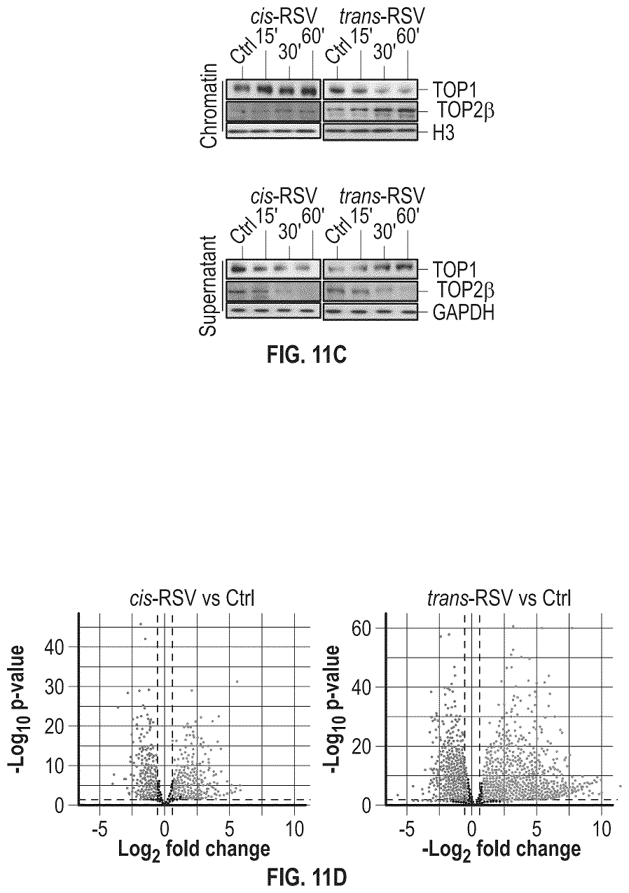

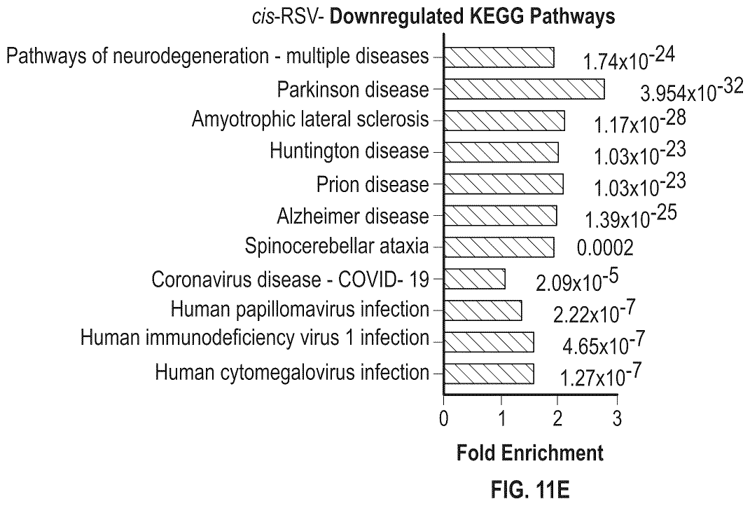

Previously it was shown that cis-RSV protects against tyrosine-mediated neurotoxicity. Therefore, we hypothesized that cis-RSV would protect against tyrosine-mediated transcription inhibition and may mimic pMAβ42 in evoking DNA damage response in presence of elevated levels of tyrosine. To test this hypothesis, we treated rat cortical neurons with either tyrosine or cis-RSV alone or in combination and quantitated the levels of EU incorporation. We found that cis-RSV itself stimulate transcriptional activation (FIG. 11A) and protect against tyrosine-mediated transcription inhibition (FIG. 11A). To determine if cis-RSV would protect against tyrosine-mediated overactivation of neuronal DNA repair (FIG. 4C), we co-treated the neuronal culture with either tyrosine, cis-and trans-RSV alone or in combination and subjected them to alkaline comet assay. We found that while cis-RSV, cither alone or in combination with tyrosine, exhibited comet tails after 16 hr, trans-RSV, alone or in combination with tyrosine, reduced them (FIG. 11B), suggesting that cis-RSV mimics the effect of picomolar Aβ42 in stimulating nuclear TyrRS localization and induction of neuroprotective DNA damage that would mimic neuronal activity-dependent DNA damage. Further, we recently showed that cis-RSV stimulates the de-PARylation of the chromatin while recruiting PARP1, and in contrast, trans-RSV is a potent stimulator of nuclear PARylation. Since picomolar Aβ42-mediated de-PARylation facilitated the degradation of TOP1 (FIG. 5E and FIG. 6B), we hypothesized that cis- and trans-RSV would have opposite effects on TOP1 and TOP2β recruitment to the chromatin. To test this hypothesis, we treated rat cortical neurons with cis-and trans-RSV for up to 1 hr and determined the presence of TOP1 and TOP2β on the chromatin. Supporting our hypothesis, we found that cis-RSV facilitated the recruitment of TOP1 along with TOP2β on the chromatin and in contrast, trans-RSV decreased TOP1 levels with concomitant recruitment of TOP2β to the chromatin (FIG. 11C).

### Example 18. Cis-RSV Downregulates Gene Expression Associated with Multiple Neurodegenerative Diseases in Primary Neurons

DSB accumulation in AD-affected neurons activates gene expression profiles with significant enrichment of genes implicated in inflammatory response with a concomitant reduction in synaptic processes and neuronal identity genes, which are common in both sporadic and familial AD. Moreover, etoposide (ETO) treatment recapitulates these DSB induced neurodegenerative signatures in primary neuron cultures, and previously it was reported that cis- and trans-RSV have differential effects on PARP1 activation and only cis-RSV protects against ETO-induced neurotoxicity in rat cortical cultures.

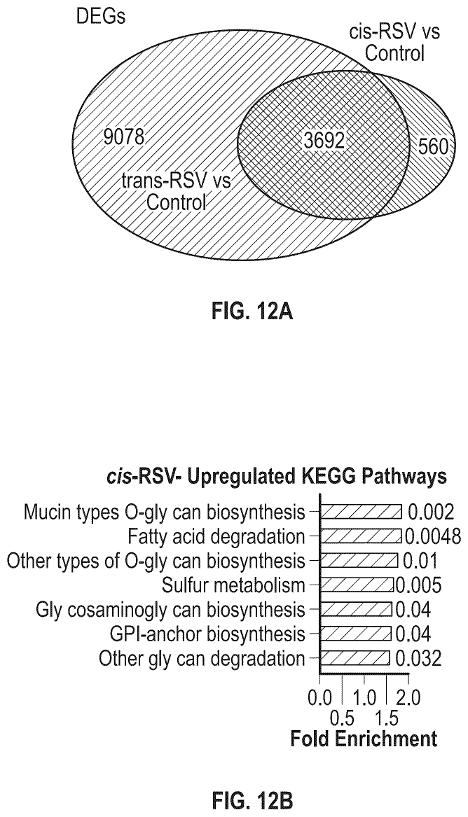

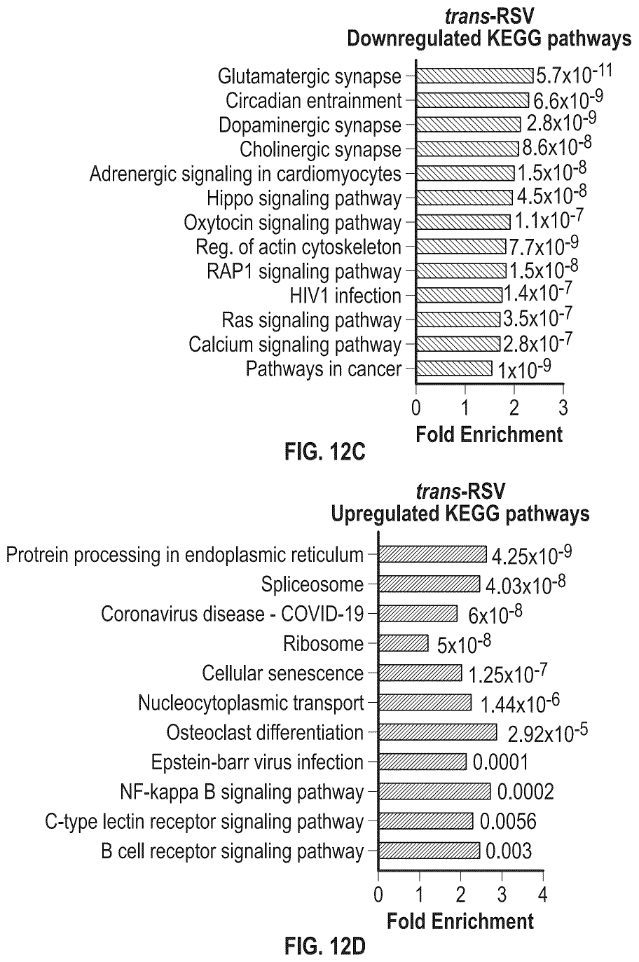

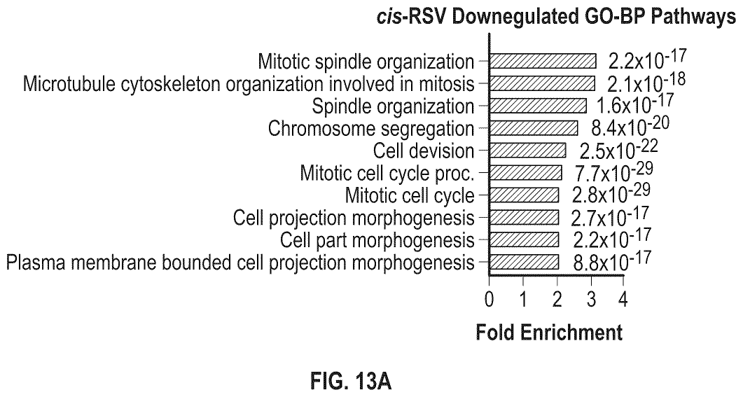

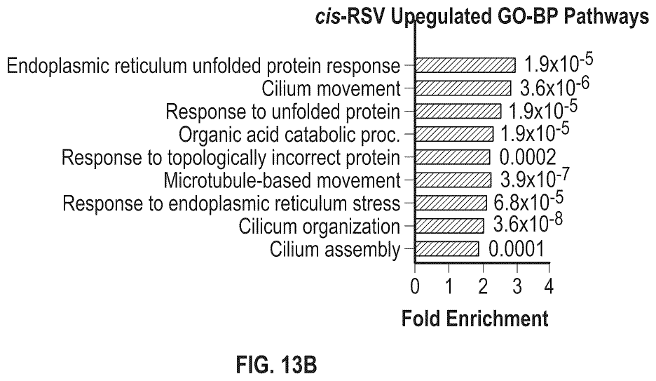

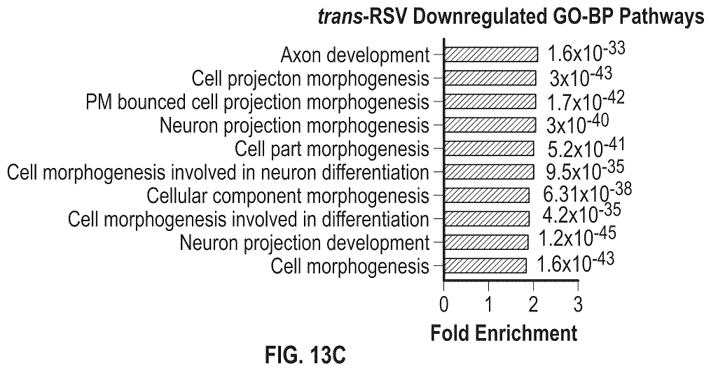

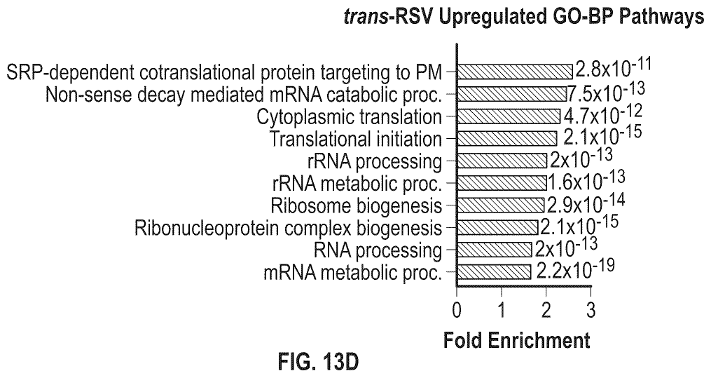

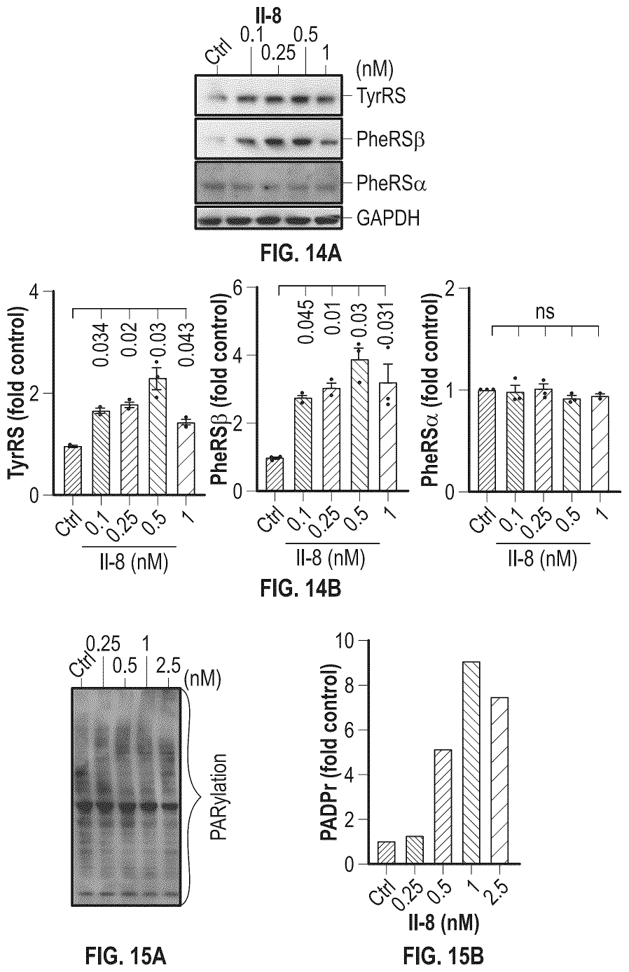

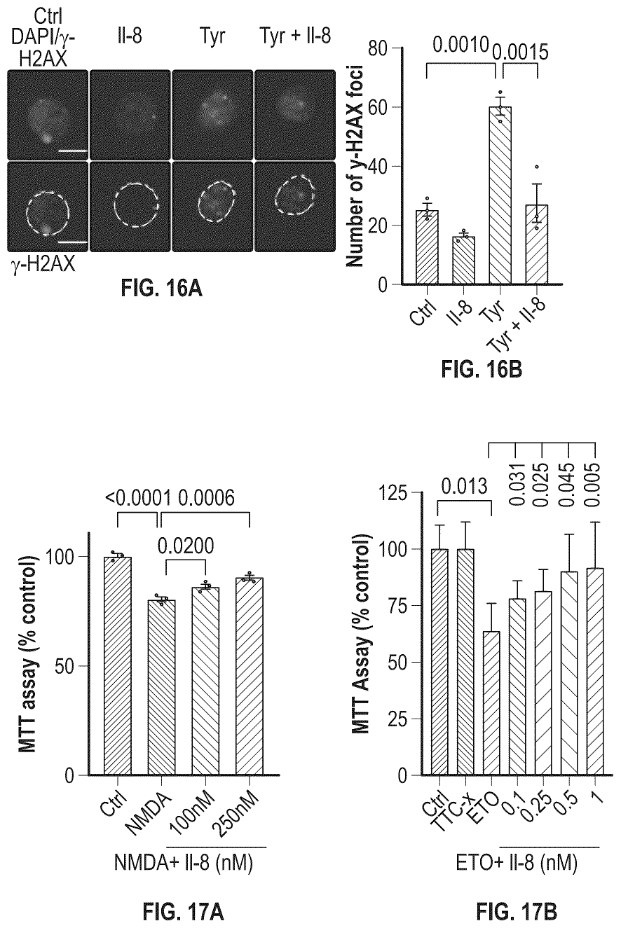

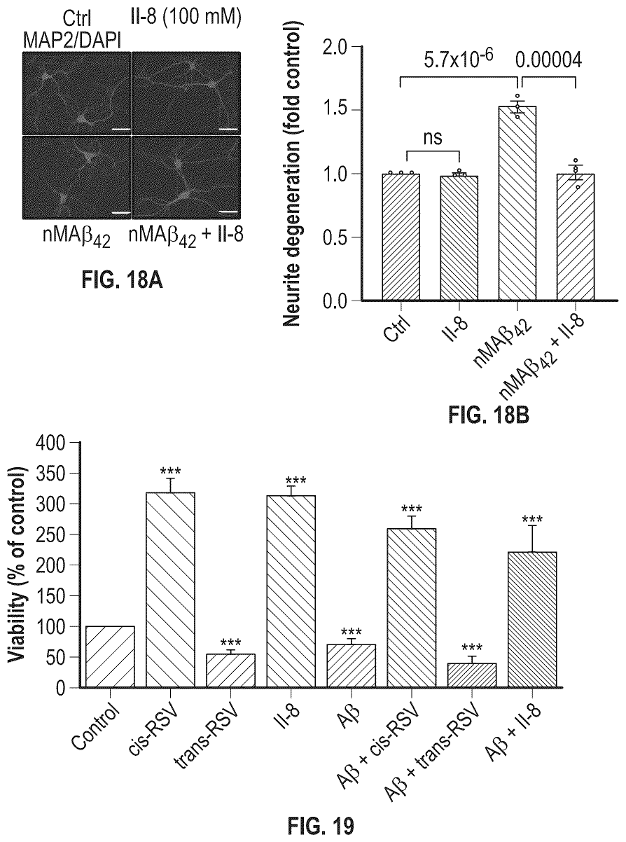

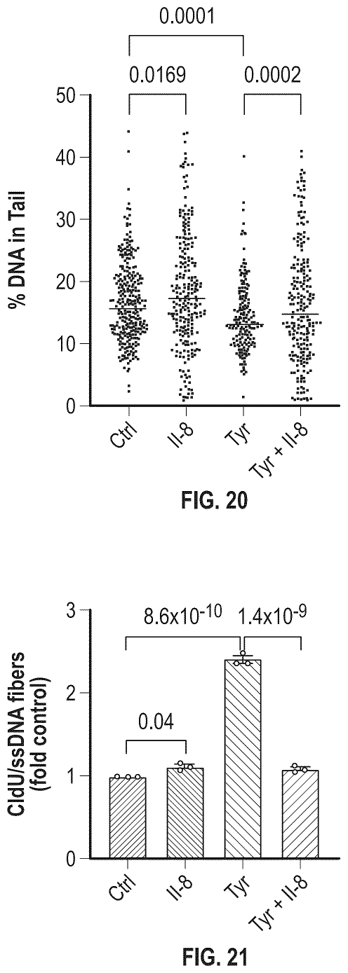

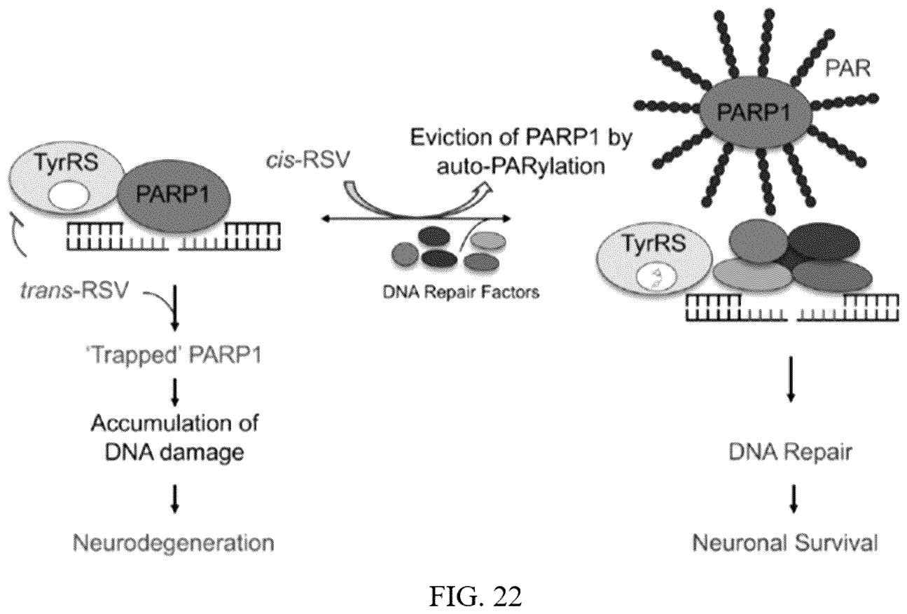

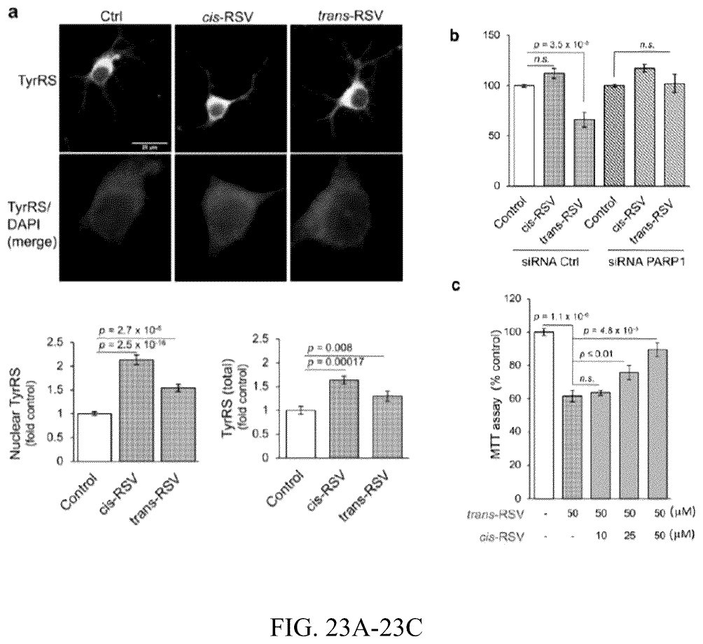

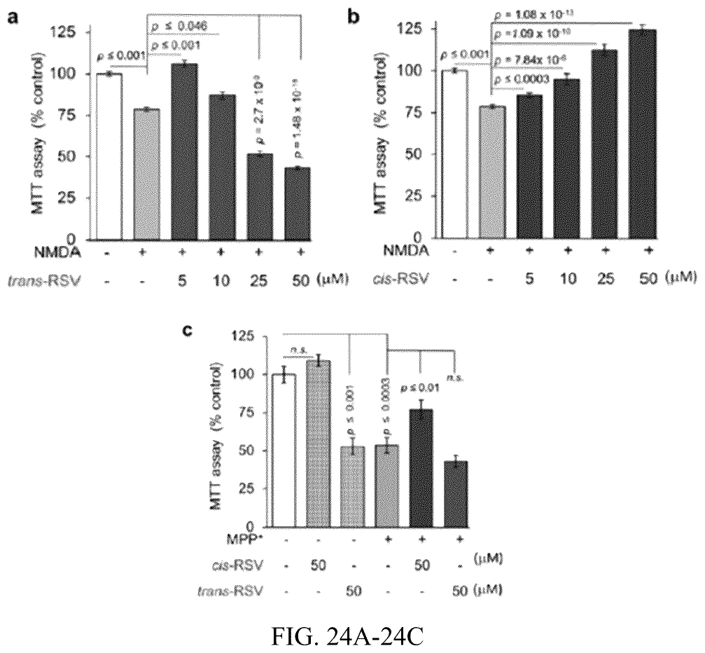

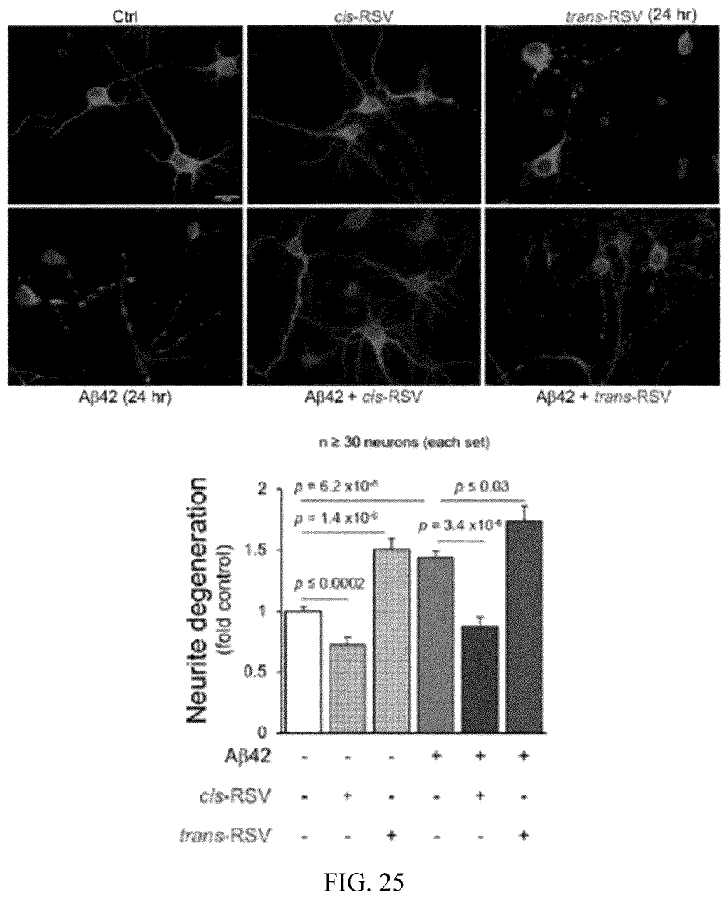

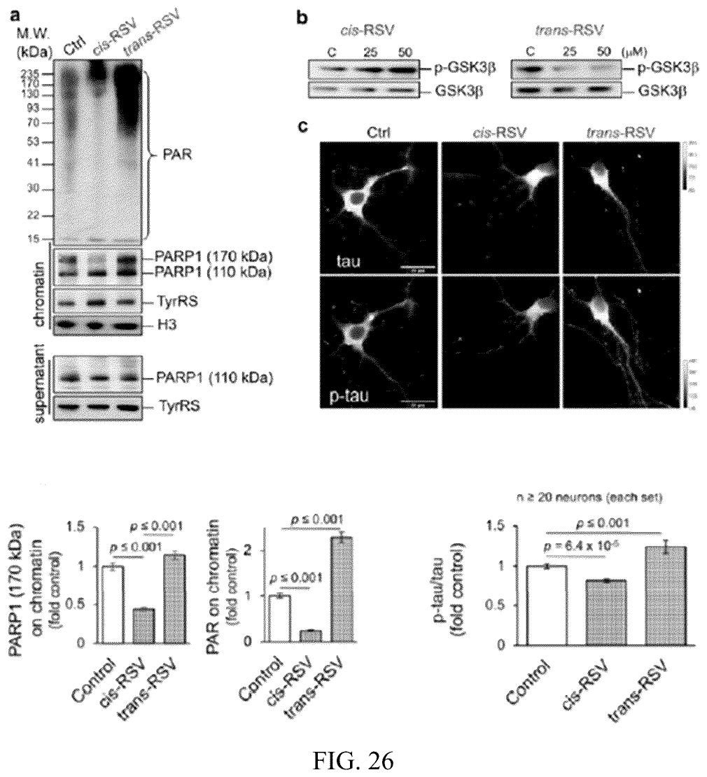

For RNA-Seq, neurons were treated with cis- and trans-RSV for 24 hr. RNA-Seq analysis showed that cis-RSV treatment modulates a smaller subset of gene expression changes compared to trans-RSV (FIG. 10D) and cis-RSV downregulated pathways associated with multiple neurodegenerative diseases and viral infections, including AD and COVID-19, suggesting that cis-RSV is a broad-spectrum neuroprotective compound (FIG. 10E). Major pathways increased with cis-RSV include O-glycan biosynthesis and fatty acid degradation (FIGS. 12A and B). Interestingly, trans-RSV downregulated pathways were associated with base excision repair, synaptic genes, and circadian entrainment (FIG. 12C). In contrast, trans-RSV upregulated NF-κB-dependent inflammatory gene expression along with pathways associated with viral infection including by coronavirus/COVID-19, ribosome biogenesis, and cellular senescence (FIG. 12D). Similar trends were observed in GO analysis as well (FIGS. 13A-D) In this context, it is interesting to note that severe COVID-19 patients accumulate serum phenylalanine showing positive correlation with inflammatory response and disease severity and exhibited brain gene expression with molecular signatures of aging and neurodegeneration.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A compound of Formula II: 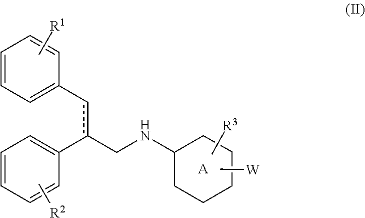
or a pharmaceutically acceptable salt thereof, wherein:
the bond [symbol] is a double or single bond;
the A ring 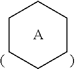
is phenyl, C₃-C₆cycloalkyl, or a 4- to 6-membered heterocyclic group;
W is 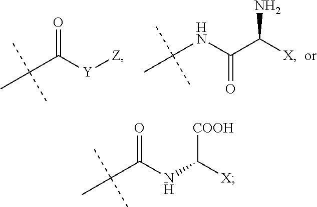
R¹ is absent or is 1 to 5 substituents independently selected from halogen, hydroxyl, amino, cyano, C₁-C₄alkyl, C₁-C₄alkoxy, (C₃-C₆cycloalkyl)C₀-C₂alkyl, C₁-C₂haloalkyl, and C₁-C₂haloalkoxy;
R² is absent or is 1 to 5 substituents independently selected from halogen, hydroxyl, amino, cyano, C₁-C₄alkyl, C₁-C₄alkoxy, (C₃-C₆cycloalkyl)C₀-C₂alkyl, C₁-C₂haloalkyl, and C₁-C₂haloalkoxy;
R³ is absent or is 1 to 4 substituents independently selected from halogen, C₁-C₂alkyl, C₁-C₂alkoxy, cyclopropyl, C₁-C₂haloalkyl, and C₁-C₂haloalkoxy;
Y is NH or O;
Z is H, hydroxyl, methyl, proline where the proline pyrrolidine ring is formed by Z and the nearest NH being joined by a —CH₂CH₂CH₂— chain, or Z is a group —CH₂X;
X is an amino acid side chain selected from H, —CH₃, —CH₂SH, —CH₂CO₂H, ₂OH, —CH(CH₃)OH, —CH₂CH₂CO₂H, —CH₂CH₂CONH₂, 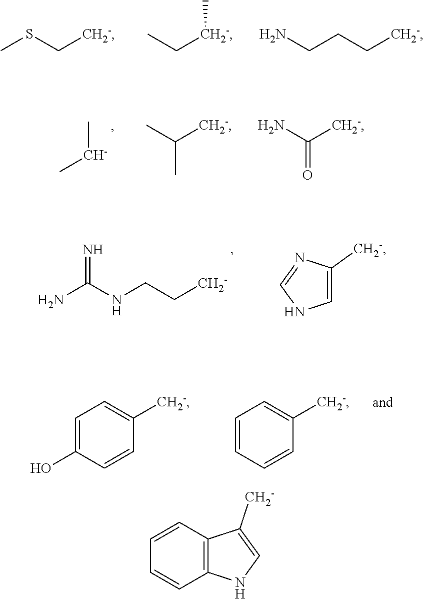
where a dash (—) indicates the point of attachment in Formula II.

2. The compound of claim 1, wherein the compound is a compound of Formula (II-a), (II-b), (II-c), or (II-d), or a pharmaceutically acceptable salt thereof, wherein Formula (II-a), (II-b), (II-c), and (II-d) are

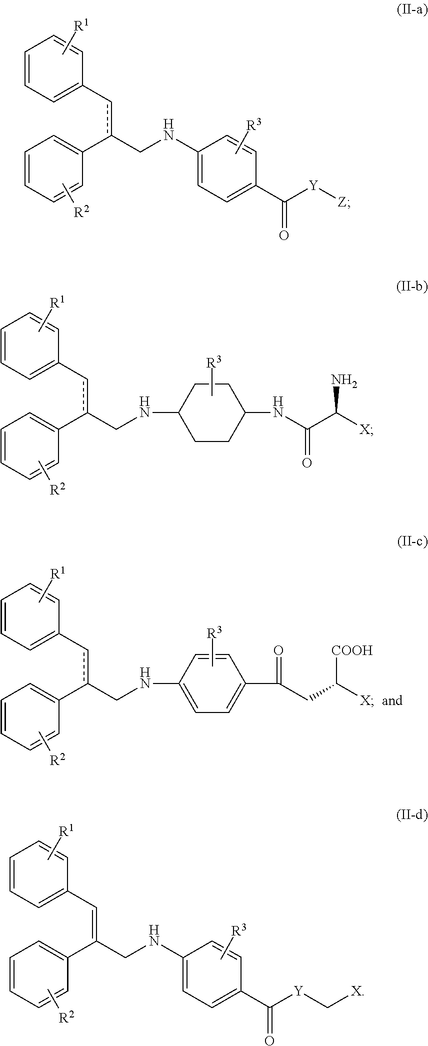

3-4. (canceled)

5. The compound of claim 1,
where R¹ and R² are each absent or are 1 to 2 substituents independently selected from halogen, hydroxyl, amino, cyano, C₁-C₄alkyl, C₁-C₄alkoxy, C₁-C₂haloalkyl, and C₁-C₂haloalkoxy; and
R³ is absent.

6-12. (canceled)

13. The compound or pharmaceutically acceptable salt thereof of claim 1, wherein the compound is selected from 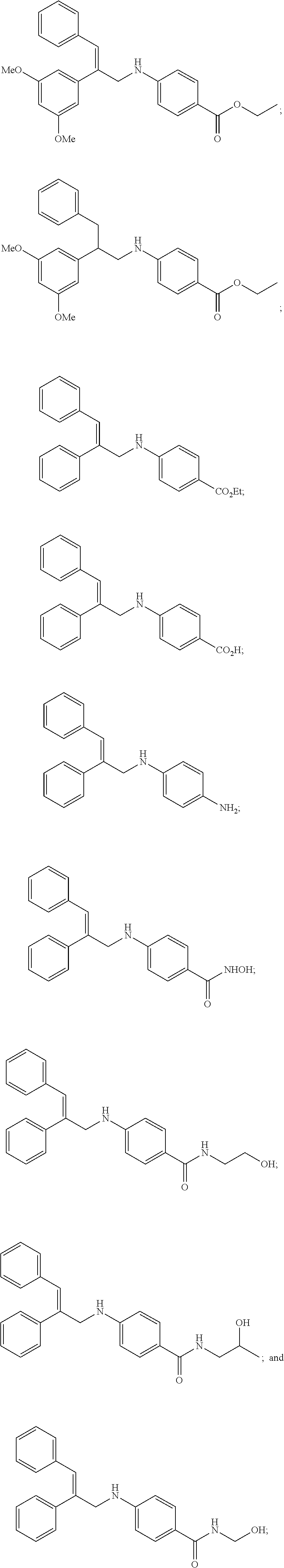
and the pharmaceutically acceptable salts of any of the foregoing.

14. (canceled)

15. The compound or pharmaceutically acceptable salt thereof of claim 1, where the compound of Formula II is:

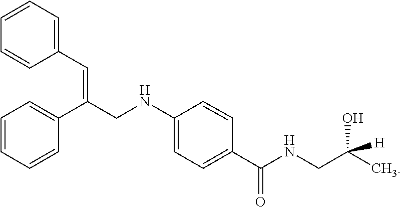 [SMILES: C[C@@H](O)CNC(=O)c1ccc(NC/C(=C/c2ccccc2)c2ccccc2)cc1]

16. A pharmaceutical composition comprising the compound or salt thereof of claim 1, together with a pharmaceutically acceptable excipient.

17-19. (canceled)

20. A method of treating a disorder in a subject, the method comprising administering a therapeutically effective amount of the compound of claim 1 to the subject.

21. The method of claim 20, wherein the disorder is a traumatic brain injury, age-related cognitive impairment, or a neurodegenerative disorder.

22. (canceled)

23. The method of claim 20, wherein the disorder is a neurodegenerative disease and the neurogenerative disease is Alzheimer's dementia, frontal cortex dementia, Lewy body dementia, multiple sclerosis, or Parkinson's disease.

24-26. (canceled)

27. The method of claim 20, wherein the disorder is a traumatic brain injury.

28-30. (canceled)

31. A method for inhibiting DNA repair, the method comprising:
contacting a cell with an effective amount of the compound of Formula II or pharmaceutically acceptable salt thereof of claim 1.

32. The method claim 31, wherein the effective amount of the compound of Formula II or salt thereof is an amount sufficient to increase an amount of TyrRS in the cell contacted with the compound of Formula II or salt thereof of, compared to an amount of TyrRS in a control cell which is not contacted with the compound of Formula II or salt thereof.

33-39. (canceled)

40. The method of claim 31, wherein the cell is a cultured cell expressing poly-ADP-ribose polymerase 1 (PARP1).

41. A pharmaceutical dosage form comprising
(i) a compound chosen of Formula II or a salt thereof of claim 1; and
(ii) a GLP-1 receptor agonist or pharmaceutically acceptable salt thereof, where the GLP-1 agonist selected from liraglutide, semaglutide, dulaglutide, exenatide, lixisenatide, tirzepatide, danuglipron (CAS Reg. No. 2230198-02-2), and orforglipron (CAS Reg. No. 2212020-52-3).

42. The pharmaceutical dosage form of claim 41, wherein the compound is a compound of Formula II is 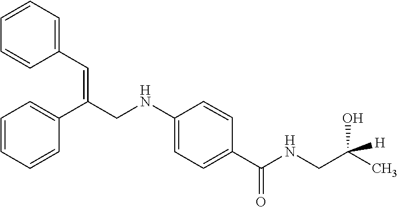 [SMILES: C[C@@H](O)CNC(=O)c1ccc(NC/C(=C/c2ccccc2)c2ccccc2)cc1]
and the GLP-1 receptor agonist is exenatide or a pharmaceutically acceptable salt thereof.

43-47. (canceled)

48. A method of treating a traumatic brain injury, age-related cognitive decline, or a neurodegenerative disorder, the method comprising administering a pharmaceutical composition of claim 41 to a patient in need of such treatment.

49. A method of treating a traumatic brain injury, a neurodegenerative disorder, or age-related cognitive impairment, the method comprising administering to a patient in need of such treatment
(i) a therapeutically effective amount of a compound of Formula II or a pharmaceutically acceptable salt thereof of claim 1; and
(ii) a therapeutically effective amount of a GLP-1 receptor agonist or pharmaceutically acceptable salt thereof, where the GLP-1 agonist selected from liraglutide, semaglutide, dulaglutide, exenatide, lixisenatide, tirzepatide, danuglipron (CAS Reg. No. 2230198-02-2), and orforglipron (CAS Reg. No. 2212020-52-3).

50-57. (canceled)

58. A method for inhibiting DNA repair, the method comprising:
contacting a cell with an effective amount of a compound of Formula I 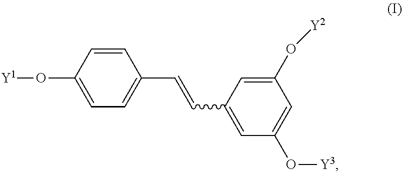
or a pharmaceutically acceptable salt thereof, wherein:
within Formula I
the bond [symbol] indicates the compound of Formula I can be in either the cis or trans conformation or a mixture of cis and trans conformations;
each of Y¹, Y², and Y³ is independently selected from a H or a group 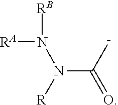
where
R is independently chosen at each occurrence from the 20 native amino acid side chains where any hydroxyl group in the amino acid side chain is optionally acylated with a C₂-C₆acyl group and when R is a proline the proline pyrrolidine ring is formed by R and R^{A} being joined by a —CH₂CH₂CH₂— chain; and
R^{A} and R^{B} are independently chosen at each occurrence from H, C₂-C₆acyl, C₁-C₆alkyl, (C₃-C₆cycloalkyl)C₀-C₄alkyl, phenyl, and benzyl.

59-64. (canceled)

65. The method of claim 58, wherein the compound is selected from: 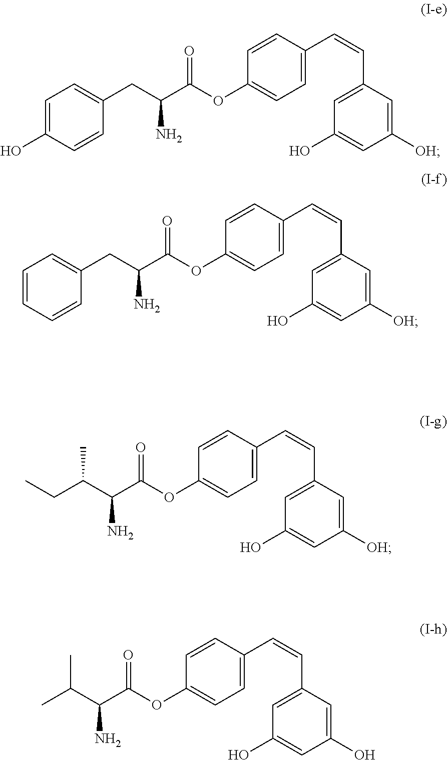 [SMILES: N[C@@H](Cc1ccc(O)cc1)C(=O)Oc1ccc(/C=C\c2cc(O)cc(O)c2)cc1 ; N[C@@H](Cc1ccccc1)C(=O)Oc1ccc(/C=C\c2cc(O)cc(O)c2)cc1 ; CC[C@H](C)[C@H](N)C(=O)Oc1ccc(/C=C\c2cc(O)cc(O)c2)cc1 ; CC(C)[C@H](N)C(=O)Oc1ccc(/C=C\c2cc(O)cc(O)c2)cc1]
and the pharmaceutically acceptable salt of any of the foregoing.

66. The method of claim 58, wherein the compound is selected from: 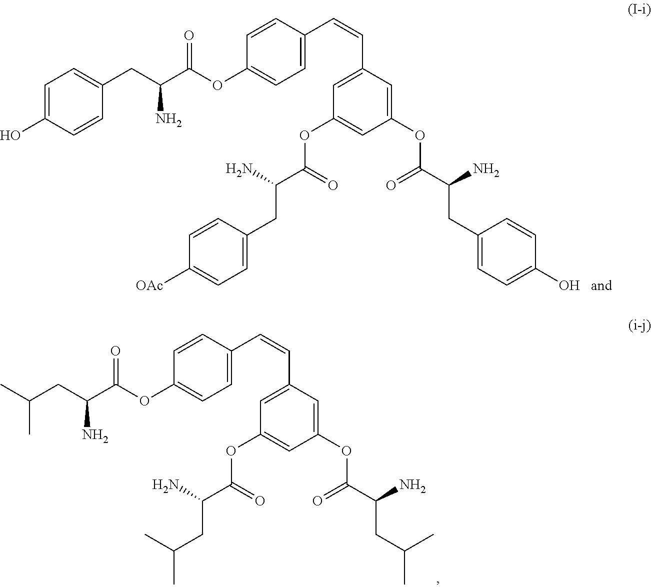
and the pharmaceutically acceptable salts of each of the foregoing.

67-71. (canceled)
